(19) United States
(12) Patent Application Publication
(10) Pub. No.: US 2011/0236827 A1
(43) Pub. Date: Sep. 29, 2011

(54) RESIN AND PHOTORESIST COMPOSITION CONTAINING THE SAME

(75) Inventors: Ichiki TAKEMOTO, Kawanishi-shi (JP); Nobuo ANDO, Toyonaka-shi (JP)

(73) Assignee: SUMITOMO CHEMICAL COMPANY, LIMITED, Tokyo (JP)

(21) Appl. No.: 13/070,190

(22) Filed: Mar. 23, 2011

(30) Foreign Application Priority Data
Mar. 26, 2010 (JP) 2010-071812

(51) Int. Cl. G03F 7/004; C07C 309/64; C07C 309/72; C07C 69/734; C08G 63/12; C08G 63/78

(52) U.S. Cl. 430/270.1; 558/52; 558/46; 560/181; 528/298

(57) ABSTRACT

The present invention provides a resin obtained by reacting a compound represented by the formula (I):

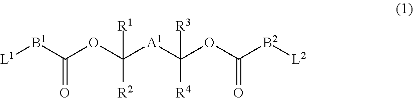

wherein R¹ represents a hydrogen atom or a C1-C6 alkyl group, R², R³ and R⁴ each independently represents a C1-C6 alkyl group and R¹, R², R³, R⁴ and A¹ are bonded to form a ring, A¹ represents a C1-C20 saturated hydrocarbon group in which one or more —CH₂— can be replaced by —O—, B¹ and B² each independently represent a C1-C6 alkylene group, L¹ and L² each independently represent a halogen atom, —O—CH═CH₂, —O—CH═CH(CH₃) or —O—SO₂—R′ in which R′ represents a C1-C6 alkyl group or a C6-C20 aryl group, with a polyhydric phenol compound.

This nonprovisional application claims priority under 35 U.S.C. §119 (a) on Patent Application No. 2010-071812 filed in JAPAN on Mar. 26, 2010, the entire contents of which are hereby incorporated by reference.

### FIELD OF THE INVENTION

The present invention relates to a novel resin and a photoresist composition containing the same.

### BACKGROUND OF THE INVENTION

A photoresist composition is used for semiconductor microfabrication.

US 2005/0014095 A1 discloses a photoresist composition containing a copolymer obtained by polymerizing 2-ethyl-2-adamantyl methacrylate with p-hydroxyxtyrene, an acid generator and a basic compound.

U.S. Pat. No. 7,494,763 B2 also discloses a chemically amplified resist composition containing a polyhydric phenol compound wherein at least one hydroxyl group bonded to a phenyl group is protected by a (2-ethyl-2-adamantyloxycarbonyl)methyl group.

### SUMMARY OF THE INVENTION

An object of the present invention is to provide a novel resin capable of providing a photoresist composition giving patterns having good sensitivity and good line edge roughness.

The other object of the present invention is to provide a photoresist composition containing the same.

These and other objects of the present invention will be apparent from the following description.

The present invention relates to the followings:

<1> A resin obtained by reacting a compound represented by the formula (I):

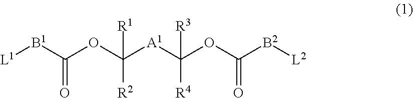

wherein R¹ represents a hydrogen atom or a C1-C6 alkyl group, R², R³ and R⁴ each independently represents a C1-C6 alkyl group and R¹, R², R³, R⁴ and A¹ are bonded to form a ring, A¹ represents a C1-C20 saturated hydrocarbon group in which one or more —CH₂— can be replaced by —O—, B¹ and B² each independently represent a C1-C6 alkylene group, L¹ and L² each independently represent a halogen atom, —O—CH═CH₂, —O—CH═CH(CH₃) or —O—SO₂—R′ in which R′ represents a C1-C6 alkyl group or a C6-C20 aryl group, with a polyhydric phenol compound;
<2> The resin according to <1>, wherein R′ is a methyl group or a tolyl group;
<3> The resin according to <1> or <2>, wherein the polyhydric phenol compound is at least one selected from the group consisting of compounds represented by the formulae (2) and (4) to (7):

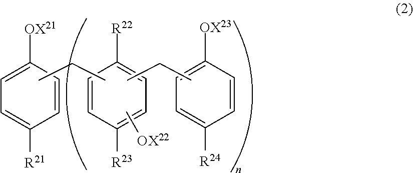

wherein R²¹, R²², R²³ and R²⁴ each independently represent a hydrogen atom, —OX²⁴ or a C1-C6 alkyl group, n represents an integer of 0 to 3, X²¹, X²², X²³ and X²⁴ each independently represent a hydrogen atom or a group represented by the formula (3):

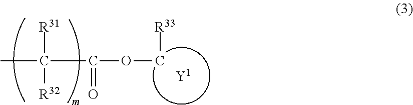

wherein R³¹ and R³² each independently represent a hydrogen atom or a C1-C6 alkyl group, m represents an integer of 1 to 4, R³³ represents a C1-C6 alkyl group or a C3-C12 saturated cyclic hydrocarbon group, and ring Y¹ represents a C3-C20 saturated hydrocarbon ring, and at least two selected from the group consisting of X²¹, X²², X²³ and X²⁴ are hydrogen atoms,

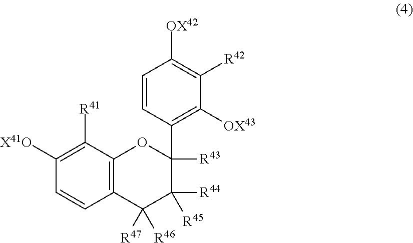

wherein R⁴¹, R⁴², R⁴³, R⁴⁴, R⁴⁵, R⁴⁶ and R⁴⁷, each independently represents a hydrogen atom, —OX⁴⁴ or a C1-C6 alkyl group, and R⁴³ and R⁴⁴ can be bonded each other to form a C3-C20 ring together with the carbon atoms to which they are bonded, and R⁴⁶ and R⁴⁷ can be bonded each other to form a C3-C20 ring together with the carbon atom to which they are bonded, X⁴¹, X⁴², X⁴³ and X⁴⁴ each independently represent a hydrogen atom or the group represented by the formula (3), and at least two selected from the group consisting of X⁴¹, X⁴², X⁴³ and X⁴⁴ are hydrogen atoms,

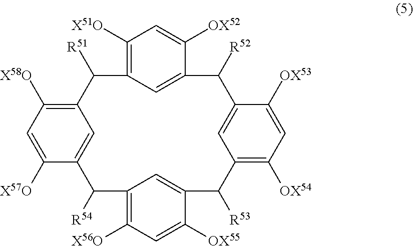

wherein R⁵¹, R⁵², R⁵³ and R⁵⁴ each independently represents a hydrogen atom, —OX⁵⁹, a C1-C6 alkyl group, a C3-C10 cycloalkyl group, a C4-C20 cycloalkylalkyl group, a C6-C20 aryl group or a C7-C20 aralkyl group, and the alkyl group, the aryl group and the aralkyl group may be substituted with —OX⁶⁰ and X⁵¹, X⁵², X⁵³, X⁵⁴, X⁵⁵, X⁵⁶, X⁵⁷, X⁵⁸, X⁵⁹ and X⁶⁰ each independently represent a hydrogen atom or the group represented by the formula (3), and at least two selected from the group consisting of X⁵¹, X⁵², X⁵³, X⁵⁴, X⁵⁵, X⁵⁶, X⁵⁷, X⁵⁸, X⁵⁹ and X⁶⁰ are hydrogen atoms,

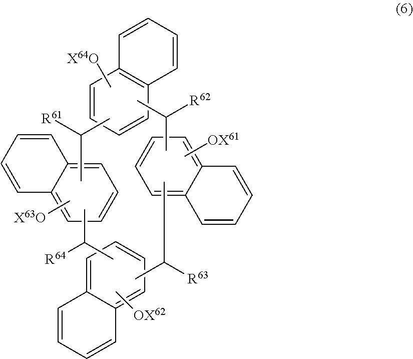

wherein R⁶¹, R⁶², R⁶³ and R⁶⁴ each independently represents a hydrogen atom, —OX⁶⁵, a C1-C6 alkyl group, a C3-C10 cycloalkyl group, a C4-C20 cycloalkylalkyl group, a C6-C20 aryl group or a C7-C20 aralkyl group, and the alkyl group, the aryl group and the aralkyl group may be substituted with —OX⁶⁶, and X⁶¹, X⁶², X⁶³, X⁶⁴, X⁶⁵ and X⁶⁶ each independently represent a hydrogen atom or the group represented by the formula (3), and at least two selected from the group consisting of X⁶¹, X⁶², X⁶³, X⁶⁴, X⁶⁵ and X⁶⁶ are hydrogen atoms,

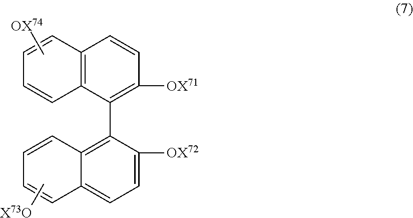

wherein X⁷¹, X⁷², X⁷³ and X⁷⁴ each independently represent a hydrogen atom or the group represented by the formula (3), and at least two selected from the group consisting of X⁷¹, X⁷², X⁷³ and X⁷⁴ are hydrogen atoms;
<4> The resin according to any one of <1> to <3>, wherein the compound represented by the formula (1) is a compound represented by the formula (10):

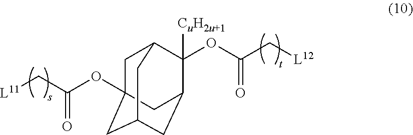

wherein s, t and u each independently represent an integer of 1 to 6, L¹¹ and L¹² each independently represent a halogen atom, —O—CH═CH₂, —O—CH═CH(CH₃) or —O—SO₂—R″ in which R″ represents a C1-C6 alkyl group or a C6-C20 aryl group;
<5> The resin according to any one of <1> to <4>, wherein the resin is obtained by reacting a compound represented by the formula (10) with a polyhydric phenol compound;
<6> A compound represented by the formula (10):

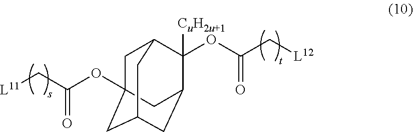

wherein s, t and u each independently represent an integer of 1 to 6, L¹¹ and L¹² each independently represent a halogen atom, —O—CH═CH₂—O—CH═CH(CH₃) or —O—SO₂—R″ in which R″ represents a C1-C6 alkyl group or a C6-C20 aryl group;
<7> A process for producing a resin comprising reacting a compound represented by the formula (I):

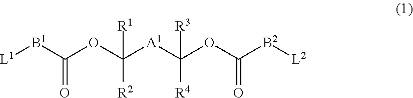

wherein R¹ represents a hydrogen atom or a C1-C6 alkyl group, R², R³ and R⁴ each independently represents a C1-C6 alkyl group and R¹, R², R³, R⁴ and A¹ are bonded to form a ring, A¹ represents a C1-C20 saturated hydrocarbon group in which one or more —CH₂— can be replaced by —O—, B¹ and B² each independently represent a C1-C6 alkylene group, L¹ and L² each independently represent a halogen atom, —O—CH═CH₂, —O—CH═CH(CH₃) or —O—SO₂—R′ in which R′ represents a C1-C6 alkyl group or a C6-C20 aryl group, with a polyhydric phenol compound;
<8> A photoresist composition comprising the resin according to any one of <1> to <5> and an acid generator;
<9> The photoresist composition according to <8>, wherein the acid generator is an acid generator represented by the formula (B1):

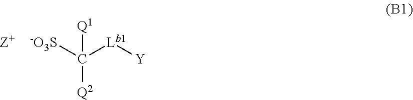

wherein Q¹ and Q² each independently represent a fluorine atom or a C1-C6 perfluoroalkyl group, L^{b1} represents a single bond or a C1-C17 saturated divalent hydrocarbon group in which one or more —CH₂— can be replaced by —O— or —CO—, Y represents a C1-C18 aliphatic hydrocarbon group or a C3-C18 saturated cyclic hydrocarbon group, and the aliphatic hydrocarbon group and the saturated cyclic hydrocarbon group can have one or more substituents, and one or more —CH₂— in the aliphatic hydrocarbon group and the saturated cyclic hydrocarbon group can be replaced by —O—, —CO— or —SO₂—, and Z⁺ represents an organic cation.

### DESCRIPTION OF PREFERRED EMBODIMENTS

First, the resin of the present invention will be illustrated.

The resin of the present invention is obtained by reacting a compound represented by the formula (I):

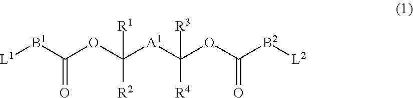

wherein R¹ represents a hydrogen atom or a C1-C6 alkyl group, R², R³ and R⁴ each independently represents a C1-C6 alkyl group and R¹, R², R³, R⁴ and A¹ are bonded to form a ring, A¹ represents a C1-C20 saturated hydrocarbon group in which one or more —CH₂— can be replaced by —O—, B¹ and B² each independently represent a C1-C6 alkylene group, L¹ and L² each independently represent a halogen atom, —O—CH═CH₂, —O—CH═CH(CH₃) or —O—SO₂—R′ in which R′ represents a C1-C6 alkyl group or a C6-C20 aryl group (hereinafter, simply referred to as COMPOUND (1)), with a polyhydric phenol compound. Hereinafter, the resin of the present invention is simply referred to as RESIN (1).

Examples of the C1-C6 alkyl group represented by R¹, R², R³ and R⁴ include a methyl group, an ethyl group, a propyl group, an isopropyl group, a butyl group, an isobutyl group, a sec-butyl group, a tert-butyl group, a pentyl group, a neopentyl group, a tert-pentyl group, a 1-methylbutyl group, a hexyl group, an isohexyl group, a 1-methylpentyl group, a 2-methylpentyl group, a 3-methylpentyl group, a 1-ethylbutyl group, a 2-ethylbutyl group, a 3-ethylbutyl group, a 1,1-dimethylbutyl group and a 1-methyl-2-ethylproypl group. R¹ is preferably a hydrogen atom, a methyl group, an ethyl group, an isopropyl group or a butyl group, and it is preferred that R², R³ and R⁴ each independently a methyl group, an ethyl group, an isopropyl group or a butyl group.

In this specification, “R¹, R², R³, R⁴ and A¹ are bonded to form a ring” means that A¹ and at least one selected from the group consisting of R¹, R², R³ and R⁴ are bonded to form a ring. Examples of the ring formed by bonding R¹, R², R³, R⁴ and A¹ include a ring formed by bonding R¹, R², R⁴ and A¹, and a ring formed by bonding R², R⁴ and A¹. Examples of the ring include a C3-C20 saturated ring such as a cyclopropane ring, a cyclobutane ring, a cyclopentane ring, a cyclohexane ring, a cycloheptane ring, a cyclooctane ring, a cyclononane ring, a cyclodecane ring, a cycloundecane ring, a cyclododecane ring, a cyclotridecane ring, a cyclotetradecane ring, a cyclopentadecane ring, a cyclohexadecane ring, a cycloheptadecane ring, a cyclooctadecane ring, a cyclononadecane ring, a cycloeicosane ring, a norbornane ring, an adamantane ring, a diamantine ring, a dimethanodecalin ring and a tetradecahydrotrimethanoanthracene ring. Among them, preferred are a cyclobutane ring, a cyclopentane ring, a cyclohexane ring, a norbornane ring, an adamantane ring, a diamantane ring and a dimethanodecalin ring, and more preferred is an adamantane ring.

Examples of the C1-C20 saturated hydrocarbon group represented by A¹ include a methylene group, an ethylene group, a trimethylene group, a tetramethylene group, a pentamethylene group, a hexamethylene group, a 1-methyltrimethylene group, a 1-methylpentamethylene group, a 2-methylpentamethylene group, a 3-methylpentamethylene group, a 1-ethyltetramethylene group, a 2-ethyltetramethylene group, a 3-ethyltetramethylene group, a 1,1-dimethyltetramethylene group, a 1-methyl-2-ethyltrimethylene group, a heptamethylene group, an octamethylene group, a nonamethylene group, a decamethylene group, an undecamethylene group, a dodecamethylene group, a tridecamethylene group, a tetradecamethylene group, a pentadecamethylene group, a hexadecamethylene group, a heptadecamethylene group, an octadecamethylene group, a nonadecamethylene group, an eicosamethylene group, a cyclopropanediyl group, a cyclobutanediyl group, a cyclopentanediyl group, a cyclohexanediyl group, a cycloheptanediyl group, a cyclooctanediyl group, a cyclononanediyl group, a cyclodecanediyl group, a norbornanediyl group, an adamantanediyl group, a bis(methylene)cyclohexane group, a bis(methylene)adamantane group, a bis(ethylene)norbornane group and a bis(trimethylene)adamantane group. One or more —CH₂— in the C1-C20 saturated hydrocarbon group can be replaced by —O—, and examples of the C1-C20 saturated hydrocarbon group in which one or more —CH₂— are replaced by —O— include a methylenedioxy group, an ethylenedioxy group and a trimethylenedioxy group. Among them, preferred are a methylene group, an ethylene group, a trimethylene group and a tetramethylene group.

Examples of the C1-C6 alkylene group represented by B¹ and B² include a methylene group, an ethylene group, a trimethylene group, a tetramethylene group, a pentamethylene group, a hexamethylene group, a 1-methyltrimethylene group, a 1-methylpentamethylene group, a 2-methylpentamethylene group, a 3-methylpentamethylene group, a 1-ethyltetramethylene group, a 2-ethyltetramethylene group, a 3-ethyltetramethylene group, a 1,1-dimethyltetramethylene group and a 1-methyl-2-ethyltrimethylene group. Among them, preferred are a methylene group, an ethylene group, a trimethylene group and a tetramethylene group.

Examples of the halogen atom represented by L¹ and L² include a fluorine atom, a chlorine atom, a bromine atom and an iodine atom, and a chlorine atom is preferable.

Examples of the C1-C6 alkyl group represented by R′ include a methyl group, an ethyl group, a propyl group, an isopropyl group, a butyl group, a pentyl group and a hexyl group, and examples of the C6-C20 aryl group represented by R′ include a phenyl group and a tolyl group, and a methyl group and a tolyl group are preferable, and a methyl group and a p-tolyl group are more preferable. It is preferred that L¹ and L² are independently halogen atoms, and it is more preferred that L¹ and L² are chlorine atoms.

Examples of COMPOUND (1) include the following.

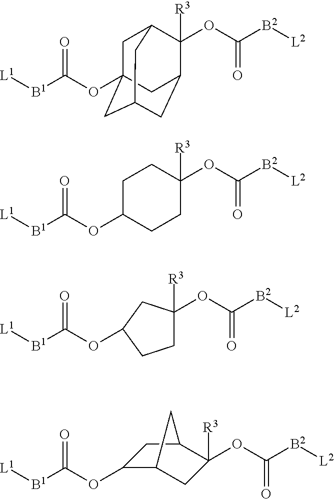

wherein R³, B¹, B², L¹ and L² are the same as defined above.

As COMPOUND (1), a compound represented by the formula:

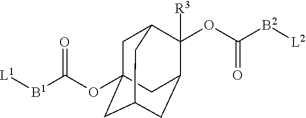

wherein R³, B¹, B², L¹ and L² are the same as defined above, is preferable, and a compound represented by the formula (10):

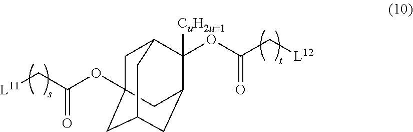

wherein s, t and u each independently represent an integer of 1 to 6, L¹¹ and L¹² each independently represent a halogen atom, —O—CH═CH₂, —O—CH═CH(CH₃) or —O—SO₂—R″ in which R″ represents a C1-C6 alkyl group or a C6-C20 aryl group, is more preferable. Examples of the C1-C6 alkyl group and the C6-C20 aryl group include the same as described in R′, and a methyl group and a tolyl group are preferable, and a methyl group and a p-tolyl group are more preferable.

COMPOUND (1) wherein B¹ and B² are the same and L¹ and L² are the same can be produced by reacting a compound represented by the formula (11):

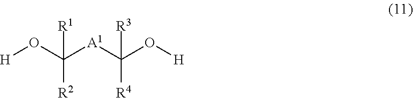

wherein R¹, R², R³, R⁴ and A¹ are the same as defined above, with a compound represented by the formula (12):

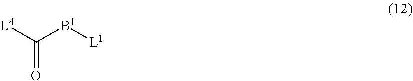

wherein L¹ and B¹ are the same as defined above, and L⁴ represents a halogen atom or —O—SO₂—R′″ in which R′″ represents a C1-C6 alkyl group or a C6-C20 aryl group. Examples of the C1-C6 alkyl group and the C6-C20 aryl group include the same as described in R′, and a methyl group and a tolyl group are preferable, and a methyl group and a p-tolyl group are more preferable.

The reaction of the compound represented by the formula (11) with the compound represented by the formula (12) is usually carried out in an inert solvent such as toluene, tetrahydrofuran, N,N-dimethylformamide and dimethylsulfoxide. The reaction temperature is usually −30 to 200° C., preferably 0 to 100° C. The used amount of the compound represented by the formula (12) is usually 2 to 4 moles and preferably 2 to 3 moles per 1 mole of the compound represented by the formula (11).

The reaction is preferably conducted in the presence of a base. Examples of the base include an organic base such as triethylamine and pyridine, and an inorganic base such as potassium carbonate and sodium hydroxide. These bases may be used alone and two or more kinds thereof may be used in combination. The used amount of the base is usually 2 to 5 moles and preferably 2 to 3 moles per 1 mole of the compound represented by the formula (11).

The reaction may be carried out in the presence of a phase transfer catalyst such as tetrabutylammonium bromide.

After completion of the reaction, COMPOUND (1) can be isolated, for example, by conducting extraction of the reaction mixture and then concentrating the organic layer obtained. COMPOUND (1) isolated may be further purified by a conventional purification means such as column chromatography, recrystallization and distillation.

The polyhydric phenol compound has one or more aromatic rings such as a benzene ring and a naphthalene ring, and has one or more phenolic hydroxyl groups on the aromatic ring.

The polyhydric phenol compound is preferably at least one selected from the group consisting of compounds represented by the formulae (2) and (4) to (7):

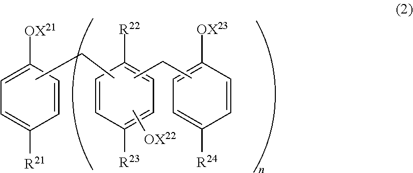

wherein R²¹, R²², R²³ and R²⁴ each independently represent a hydrogen atom, —OX²⁴ or a C1-C6 alkyl group, n represents an integer of 0 to 3, X²¹, X²², X²³ and X²⁴ each independently represent a hydrogen atom or a group represented by the formula (3):

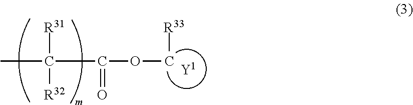

wherein R³¹ and R³² each independently represent a hydrogen atom or a C1-C6 alkyl group, m represents an integer of 1 to 4, R³³ represents a C1-C6 alkyl group or a C3-C12 saturated cyclic hydrocarbon group, and ring Y¹ represents a C3-C20 saturated hydrocarbon ring, and at least one selected from the group consisting of X²¹, X²², X²³ and X²⁴ is a hydrogen atom,

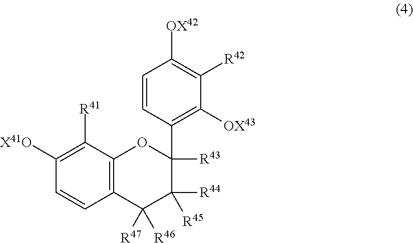

wherein R⁴¹, R⁴², R⁴³, R⁴⁴, R⁴⁵, R⁴⁶ and R⁴⁷ each independently represents a hydrogen atom, —OX⁴⁴ or a C1-C6 alkyl group, and R⁴³ and R⁴⁴ can be bonded each other to form a C3-C20 ring together with the carbon atoms to which they are bonded, and R⁴⁶ and R⁴⁷ can be bonded each other to form a C3-C20 ring together with the carbon atom to which they are bonded, X⁴¹, X⁴², X⁴³ and X⁴⁴ each independently represent a hydrogen atom or the group represented by the formula (3), and at least one selected from the group consisting of X⁴¹, X⁴², X⁴³ and X⁴⁴ is a hydrogen atom,

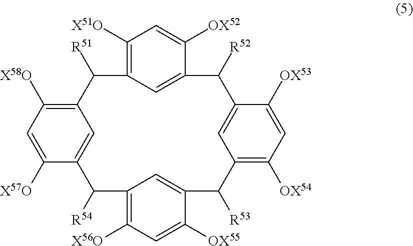

wherein R⁵¹, R⁵², R⁵³ and R⁵⁴ each independently represents a hydrogen atom, —OX⁵⁹, a C1-C6 alkyl group, a C3-C10 cycloalkyl group, a C4-C20 cycloalkylalkyl group, a C6-C20 aryl group or a C7-C20 aralkyl group, and the alkyl group, the aryl group and the aralkyl group may be substituted with —OX⁶⁰, and X⁵¹, X⁵², X⁵³, X⁵⁴, X⁵⁵, X⁵⁶, X⁵⁷, X⁵⁸, X⁵⁹ and X⁶⁰ each independently represent a hydrogen atom or the group represented by the formula (3), and at least one selected from the group consisting of X⁵¹, X⁵², X⁵³, X⁵⁴, X⁵⁵, X⁵⁶, X⁵⁷, X⁵⁸, X⁵⁹ and X⁶⁰ is a hydrogen atom,

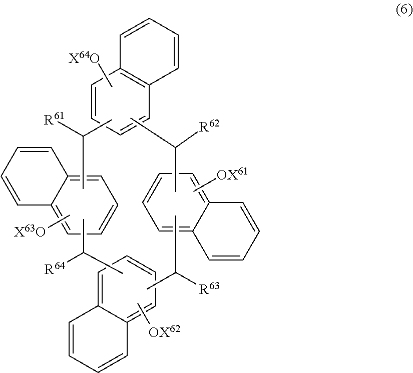

wherein R⁶¹, R⁶², R⁶³ and R⁶⁴ each independently represents a hydrogen atom, —OX⁶⁵, a C1-C6 alkyl group, a C3-C10 cycloalkyl group, a C4-C20 cycloalkylalkyl group, a C6-C20 aryl group or a C7-C20 aralkyl group, and the alkyl group, the aryl group and the aralkyl group may be substituted with —OX⁶⁶, and X⁶¹, X⁶², X⁶³, X⁶⁴, X⁶⁵ and X⁶⁶ each independently represent a hydrogen atom or the group represented by the formula (3), and at least one selected from the group consisting of X⁶¹, X⁶², X⁶³, X⁶⁴, X⁶⁵ and X⁶⁶ is a hydrogen atom,

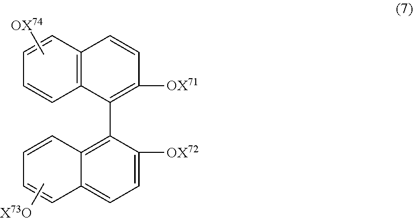

wherein X⁷¹, X⁷², X⁷³ and X⁷⁴ each independently represent a hydrogen atom or the group represented by the formula (3), and at least one selected from the group consisting of X⁷¹, X⁷², X⁷³ and X⁷⁴ is a hydrogen atom.

In the formula (2), examples of the C1-C6 alkyl group represented by R²¹, R²², R²³ and R²⁴ include the same as described above, and preferred are a methyl group, an ethyl group, an isopropyl group and a butyl group. In the formula (2), n is preferably 2.

In the formula (3), examples of the C1-C6 alkyl group represented by R³¹ and R³² include the same as described above, and it is preferred that R³¹ and R³² each independently represent a hydrogen atom or a methyl group. Examples of the C1-C6 alkyl group represented by R³³ include the same as described above, and preferred are a methyl group, an ethyl group and an isopropyl group. Examples of the ring Y¹ include the same as described in the above-mentioned C3-C20 saturated ring, and a cyclohexane ring and an adamantane ring are preferable.

Examples of the group represented by the formula (3) include a group represented by the formula (3-1):

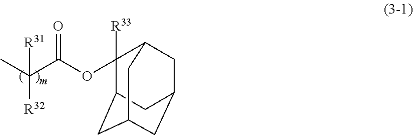

wherein R³¹, R³², R³³ and m are the same as defined above.

It is preferred that at least two selected from the group consisting of X²¹, X²², X²³ and X²⁴ are hydrogen atoms.

Examples of the compound represented by the formula (2) having no group represented by the formula (3) include the following compound represented by the formula (2-1).

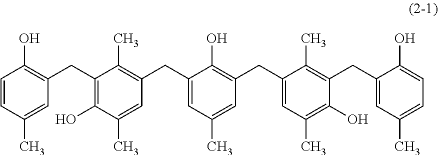

In the formula (4) examples of the C1-C6 alkyl group represented by R⁴¹, R⁴², R⁴³, R⁴⁴, R⁴⁵, R⁴⁶ and R⁴⁷ include the same as described above, and preferred are a methyl group, an ethyl group, an isopropyl group and a butyl group. Examples of the C3-C20 ring formed by bonding R⁴³ and R⁴⁴ each other together with the carbon atoms to which they are bonded and the C3-C20 ring formed by bonding R⁴⁶ and R⁴⁷ each other together with the carbon atom to which they are bonded include a cyclopropane ring, a cyclobutane ring, a cyclopentane ring, a cyclohexane ring, a cycloheptane ring, a cyclooctane ring, a cyclononane ring, a cyclodecane ring, a cycloundecane ring, a cyclododecane ring and a cyclotridecane ring, and a cyclobutane ring, a cyclopentane ring and a cyclohexane ring are preferred.

It is preferred that at least two selected from the group consisting of X⁴¹, X⁴², X⁴³ and X⁴⁴ are hydrogen atoms.

Examples of the compound represented by the formula (4) having no group represented by the formula (3) include the following compounds represented by the formulae (4-1) to (4-4).

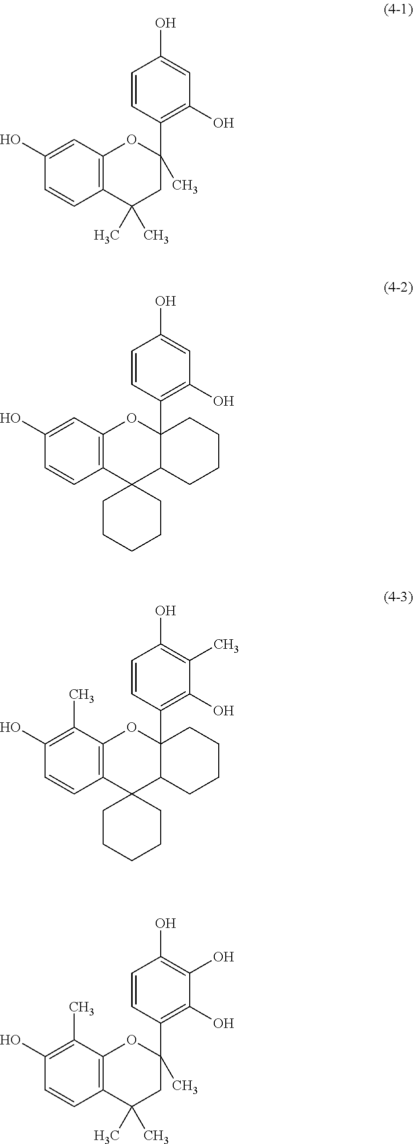

In the formula (5), examples of the C1-C6 alkyl group represented by R⁵¹, R⁵², R⁵³ and R⁵⁴ include the same as described above, and preferred are a methyl group, an ethyl group, an isopropyl group and a butyl group. Examples of the C3-C10 cycloalkyl group represented by R⁵¹, R⁵², R⁵³ and R⁵⁴ include a cyclopropyl group, a cyclobutyl group, a cyclopentyl group, a cyclohexyl group, a cycloheptyl group, a cyclooctyl group, a cyclononyl group and a cyclodecyl group. Examples of the C4-C20 cycloalkylalkyl group represented by R⁵¹, R⁵², R⁵³ and R⁵⁴ include a cyclopropylmethyl group, a cyclopropylethyl group, a cyclobutylmethyl group, a cyclobutylpropyl group, a cyclopentylmethyl group, a cyclopentylethyl group, a cyclopentylbutyl group, a cyclohexylmethyl group, a cyclohexylethyl group, a cyclohexylbutyl group, a cyclohexylpentyl group, a cyclohexylhexyl group, a cycloheptyloctyl group, a cyclooctyldecyl group, a cyclononyldodecyl group and a cyclodecylheptyl group.

Examples of the C6-C20 aryl group represented by R⁵¹, R⁵², R⁵³ and R⁵⁴ include a phenyl group and a naphthyl group, and examples of the C7-C20 aralkyl group represented by R⁵¹, R⁵², R⁵³ and R⁵⁴ include a benzyl group and a phenylethyl group. Examples of the C1-C6 alkyl group substituted with —OH represented by R⁵¹, R⁵², R⁵³ and R⁵⁴ include a hydroxymethyl group, a hydroxyethyl group, a hydroxypropyl group, a hydroxypentyl group, a hydroxyhexyl group and a 3-hydrorxybutyl group. Examples of the C6-C20 aryl group substituted with —OH represented by R⁵¹, R⁵², R⁵³ and R⁵⁴ include a hydroxyphenyl group, a dihydrorxyphenyl group and a trihydroxyphenyl group. Examples of the C7-C20 aralkyl group substituted with —OH represented by R⁵¹, R⁵², R⁵³ and R⁵⁴ include a hydroxybenzyl group.

It is preferred that R⁵¹, R⁵², R⁵³ and R⁵⁴ each independently represent a hydrogen atom, amethyl group, an ethyl group, an isopropyl group, a butyl group, a hydroxymethyl group, a hydroxyethyl group, a cyclopentyl group, a cyclohexyl group, a phenyl group, a hydroxyphenyl group or a dihydroxyphenyl group.

It is preferred that at least two selected from the group consisting of X⁵¹, X⁵², X⁵³, X⁵⁴, X⁵⁵, X⁵⁶, X⁵⁷, X⁵⁸, X⁵⁹, X⁶⁰ and X⁶¹ are hydrogen atoms.

Examples of the compound represented by the formula (5) having no group represented by the formula (3) include the following compounds represented by the formulae (5-1) to (5-4).

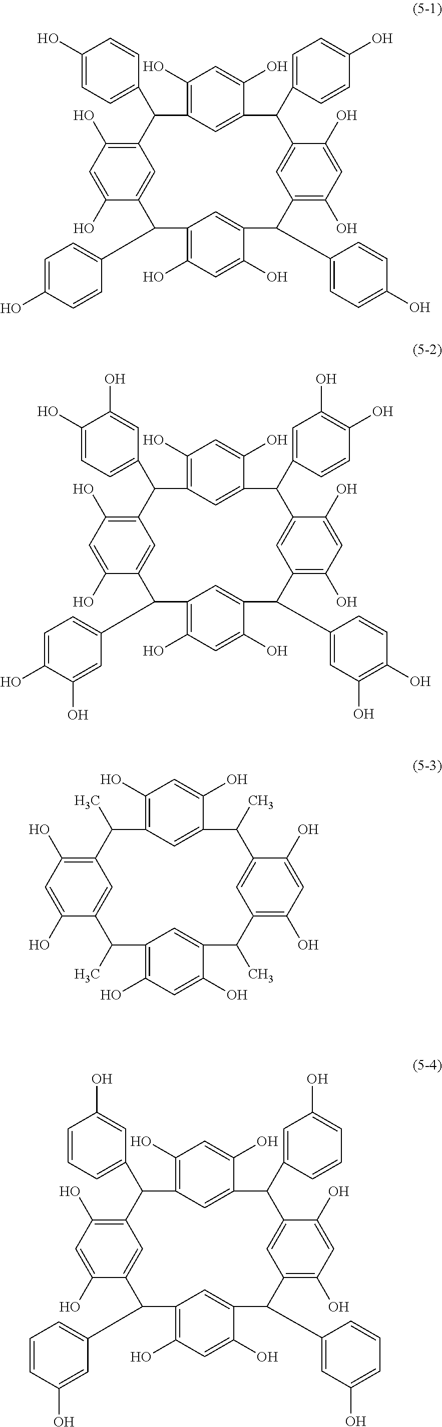

In the formula (6), examples of the C1-C6 alkyl group represented by R⁶¹, R⁶², R⁶³ and R⁶⁴ include the same as described above, and preferred are a methyl group, an ethyl group, an isopropyl group and a butyl group. Examples of the C3-C10 cycloalkyl group represented by R⁶¹, R⁶², R⁶³ and R⁶⁴ include the same as described above. Examples of the C4-C20 cycloalkylalkyl group represented by R⁶¹, R⁶², R⁶³ and R⁶⁴ include the same as defined above.

Examples of the C6-C20 aryl group represented by R⁶¹, R⁶², R⁶³ and R⁶⁴ include the same as described above, and examples of the C7-C20 aralkyl group represented by R⁶¹, R⁶², R⁶³ and R⁶⁴ include the same as described above. Examples of the C1-C6 alkyl group substituted with —OH represented by R⁶¹, R⁶², R⁶³ and R⁶⁴ include the same as described above.

It is preferred that at least two selected from the group consisting of X⁶¹, X⁶², X⁶³, X⁶⁴, X⁶⁵ and X⁶⁶ are hydrogen atoms.

Examples of the compound represented by the formula (6) having no group represented by the formula (3) include the following compound represented by the formula (6-1).

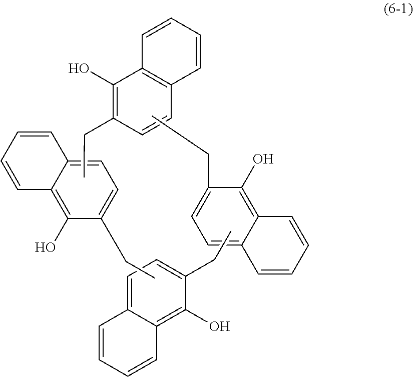

It is preferred that at least two selected from the group consisting of X⁷¹, X⁷², X⁷³ and X⁷⁴ are hydrogen atoms.

Examples of the compound represented by the formula (7) having no group represented by the formula (3) include the following compound represented by the formula (7-1).

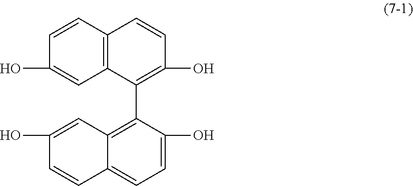

The polyhydric phenol compound having one or more groups represented by the formula (3) can be produced by reacting a compound represented by the formula (2), (4), (5), (6) or (7) and having no group represented by the formula (3) with a compound represented by the formula (3′):

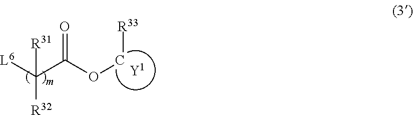

wherein R³¹, R³², R³³, Y¹ and m are the same as defined above, and L⁶ represents a halogen atom, a methylsulfonyloxy group or a tolylsulfonyloxy group. The polyhydric phenol compound having one or more groups represented by the formula (3) can be also produced by reacting a compound represented by the formula (2), (4), (5), (6) or (7) and having one or more groups represented by the formula (3) with the compound represented by the formula (3′).

The reaction of the compound represented by the formula (2), (4), (5), (6) or (7) and having no group represented by the formula (3) with a compound represented by the formula (3′) is usually conducted in an inert solvent such as toluene, tetrahydrofuran, N,N-dimethylformamide and dimethylsulfoxide. The reaction temperature is usually −30 to 200° C., preferably 0 to 150° C.

The used amount of the compound represented by the formula (3′) is usually 1 to (n−1) moles and preferably 1 to (n−2) moles per n moles of —OH in the compound represented by the formula (2), (4), (5), (6) or (7) and having no group represented by the formula (3). The reaction is preferably carried out in the presence of a base. Examples of the base include an organic base such as triethylamine, pyridine, sodium methoxide, sodium ethoxide and potassium tert-butoxide; an inorganic base such as sodium hydride, potassium carbonate and sodium hydroxide. These bases may be used alone and a mixture thereof may be used. The used amount of the base is usually 1 to 6 moles and preferably 1 to 4 moles per 1 mole of the compound represented by the formula (3′). The reaction may be conducted in the presence of a phase transfer catalyst such as tetrabutylammonium bromide.

After completion of the reaction, the polyhydric phenol compound having one or more groups represented by the formula (3) can be isolated, for example, by conducting extraction of the reaction mixture and then concentrating the organic layer obtained. The polyhydric phenol compound having one or more groups represented by the formula (3) isolated may be further purified by a conventional purification means such as column chromatography, recrystallization and distillation.

The molecular weight of the polyhydric phenol compound is usually 100 to 5,000, preferably 200 to 4,500 and more preferably 300 to 4,000.

RESIN (1) can be produced by a condensation reaction of COMPOUND (1) wherein L¹ and L² each independently represent a halogen atom or —O—SO₂—R′ and the polyhydric phenol compound. The condensation reaction is usually conducted in an inert solvent such as toluene, tetrahydrofuran, N,N-dimethylformamide and dimethylsulfoxide. The condensation reaction temperature is usually −30 to 200° C., preferably 0 to 150° C. The used amount of COMPOUND (1) is usually 1 to n moles and preferably 1 to n/2 moles per n moles of —OH in the polyhydric phenol compound. The condensation reaction is preferably carried out in the presence of a base. Examples of the base include an organic base such as triethylamine, pyridine, sodium methoxide, sodium ethoxide and potassium tert-butoxide; an inorganic base such as sodium hydride, potassium carbonate and sodium hydroxide. These bases may be used alone and a mixture thereof may be used. The used amount of the base is usually 1 to 2 n moles and preferably 1 to n/2 moles per n moles of —OH in the polyhydric phenol compound. The reaction may be conducted in the presence of a phase transfer catalyst such as tetrabutylammonium bromide.

RESIN (1) can be produced by an addition reaction of COMPOUND (1) wherein L¹ and L² each independently represent —O—CH═CH₂ or —O—CH═CH(CH₃) and the polyhydric phenol compound. The addition reaction is usually conducted in an inert solvent such as toluene, tetrahydrofuran, N,N-dimethylformamide and dimethylsulfoxide. The condensation reaction temperature is usually −70 to 200° C., preferably −20 to 150° C. The used amount of COMPOUND (1) is usually 1 to n moles and preferably 1 to n/2 moles per n moles of —OH in the polyhydric phenol compound. The addition reaction is preferably carried out in the presence of an acid. Examples of the acid include an organic acid such as acetic acid, methanesulfonic acid, benzenesulfonic acid and p-toluenesulfonic acid, an inorganic acid such as hydrochloric acid and sulfuric acid, and an acidic resin such as Nafion (trade mark registered by Dupont). These acids may be used alone and a mixture thereof may be used. The used amount of the acid is usually 1 to 2 n moles and preferably 1 to n/2 moles per n moles of —OH in the polyhydric phenol compound.

After completion of the condensation reaction or the addition reaction, RESIN (1) can be isolated, for example, by conducting extraction of the reaction mixture and then concentrating the organic layer obtained. RESIN (1) isolated may be further purified by a conventional purification means such as column chromatography, recrystallization and distillation.

The molecular weight of RESIN (1) is usually 3.0×10² to 2.0×10⁵, preferably 5.0×10² to 1.0×10⁵ and more preferably 2.0×10³ to 2.0×10⁴.

Next, the photoresist composition of the present invention will be illustrated.

RESIN (1) itself is insoluble or poorly soluble in an aqueous alkali solution and becomes soluble in an aqueous alkali solution by the action of an acid.

The photoresist composition of the present invention contains RESIN (1) and an acid generator, and it is suitable composition for extreme ultraviolet (EUV) lithography and electron beam (EB) lithography.

The content of RESIN (1) in the photoresist composition of the present invention is usually 50 to 99.9% by weight, preferably 60 to 99% by weight and more preferably 60 to 97% by weight based on 100% by weight of the solid component. In this specification, “solid component” means components other than solvent in the photoresist composition. The content can be measured according to known analytical methods. The content of the acid generator in the photoresist composition of the present invention is usually 0.1 to 50% by weight, preferably 1 to 40% by weight and more preferably 3 to 40% by weight based on 100% by weight of the solid component.

The acid generator generates an acid with the action of radiation, and the acid generated by irradiation to the photoresist composition of the present invention catalytically acts against RESIN (1), cleaves the group capable of being cleaved by the acid, and RESIN (1) becomes soluble in an alkali aqueous solution.

Examples of the photoacid generator include nonionic photoacid generators and ionic photoacid generators. Examples of the nonionic photoacid generator include organic halides, sulfonate esters such as 2-nitrobenzyl ester, aromatic sulfonate, oxime sulfonate, N-sulfonyloxyimide, sulfonyloxyketone and DNQ 4-sulfonate, and sulfones such as disulfone, ketosulfone and sulfonyldiazomethane. Examples of the ionic photoacid generator include onium salts such as a diazonium salt, a phosphonium salt, a sulfonium salt and an iodonium salt, and examples of the anion of the onium salt include sulfonic acid anion, sulfonylimide anion and sulfonylmethide anion. Examples of the onium salt include triphenylsulfonium 2,4,6-triisopropylbenzenesulfonate.

Examples of the acid generator include acid generators described in JP 63-26653A, JP 55-164824 A, JP 62-69263A, JP 63-146038 A, JP 63-163452 A, JP 62-153853 A, JP 63-146029 A, U.S. Pat. No. 3,779,778, U.S. Pat. No. 3,849,137, DE Patent No. 3914407 and EP Patent No. 126,712.

A fluorine-containing acid generator is preferable.

Preferable examples of the acid generator include an acid generator represented by the formula (B1).

In the formula (B1), examples of the C1-C6 perfluoroalkyl group include a trifluoromethyl group, a pentafluoroethyl group, a heptafluoropropyl group, a nonafluorobutyl group, an undecafluoropentyl group and a tridecafluorohexyl group, and a trifluoromethyl group is preferable. Q¹ and Q² independently preferably represent a fluorine atom or a trifluoromethyl group, and Q¹ and Q² are more preferably fluorine atoms.

Examples of the C1-C17 divalent saturated hydrocarbon group include a C1-C17 linear alkanediyl group such as a methylene group, an ethylene group, a propane-1,3-diyl group, a propane-1,2-diyl group, a butane-1,4-diyl group, a butane-1,3-diyl group, a pentane-1,5-diyl group, a hexane-1,6-diyl group, a heptane-1,7-diyl group, an octane-1,8-diyl group, a nonane-1,9-diyl group, a decane-1,10-diyl group, a undecane-1,1′-diyl group, a dodecane-1,12-diyl group, a tridecane-1,13-diyl group, a tetradecane-1,14-diyl group, a pentadecane-1,15-diyl group, a hexadecane-1,16-diyl group and a heptadecane-1,17-diyl group; a C2-C17 branched alkanediyl group such as a 1-methyl-1,3-propylene group, a 2-methyl-1,3-propylene group, a 2-methyl-1,2-propylene group, a 1-methyl-1,4-butylene group, and a 2-methyl-1,4-butylene group; a divalent monocyclic saturated hydrocarbon group such as a cyclobutane-1,3-diyl group, a cyclopentane-1,3-diyl group, a cyclohexane-1,2-diyl group, a 1-methylcyclohexane-1,2-diyl group, a cyclohexane-1,4-diyl group, a cyclooctane-1,2-diyl group and a cyclooctane-1,5-diyl group; a divalent polycyclic saturated hydrocarbon group such as a norbornane-2,3-diyl group, a norbornane-1,4-diyl group, a norbornane-2,5-diyl group, an adamantane-1,2-diyl group, an adamantane-1,5-diyl group and an adamantane-2,6-diyl group; and a group formed by combining two or more groups selected from the group consisting of the above-mentioned groups.

Examples of the C1-C17 divalent saturated hydrocarbon group in which one or more —CH₂— are replaced by —O— or —CO— include *—CO—O-L^{b2}-, *—CO—O-L^{b4}-CO—O-L^{b3}-, *-L^{b5}-O—CO—, *-L^{b7}-O-L^{b6}-, *—CO—O-L^{b8}-O—, and *—CO—O-L^{b10}-O-L^{b9}-CO—O—, wherein L^{b2} represents a single bond or a C1-C15 saturated hydrocarbon group, L^{b3} represents a single bond or a C1-C12 saturated hydrocarbon group, L^{b4} represents C1-C13 saturated hydrocarbon group, with the proviso that total carbon number of L^{b3} and L^{b4} is 1 to 13, L^{b5} represents a C1-C15 saturated hydrocarbon group, L^{b6} represents a C1-C15 saturated hydrocarbon group, L^{b7} represents a C1-C15 saturated hydrocarbon group, with the proviso that total carbon number of L^{b6} and L^{b7} is 1 to 16, L^{b8} represents a C1-C14 saturated hydrocarbon group, L^{b9} represents a C1-C11 saturated hydrocarbon group, L^{b10} represents a C1-C11 saturated hydrocarbon group, with the proviso that total carbon number of L^{b9} and L^{b10} is 1 to 12, and * represents a binding position to —C(Q¹)(Q²)-. Among them, preferred is *—CO—O-L^{b2}-, and more preferred is *—CO—O-L^{b2}- in which L^{b2} is a single bond or —CH₂—.

Examples of *—CO—O-L^{b2}- include *—CO—O— and *—CO—O—CH₂—. Examples of *—CO—O-L^{b4}-CO—O-L^{b3}- include *—CO—O—CH₂—CO—O—, *—CO—O—(CH₂)₂—CO—O—, *—CO—O—(CH₂)₃—CO—O—, *—CO—O—(CH₂)₄—CO—O—, *—CO—O—(CH₂)₆—CO—O—, *—CO—O—(CH₂)₈—CO—O—, *—CO—O—CH₂—CH(CH₃)—CO—O— and *—CO—O—CH₂—C(CH₃)₂—CO—O—. Examples of *-L^{b5}-O—CO— include *—CH₂—O—CO—, *—(CH₂)₂—O—CO—, *—(CH₂)₃—O—CO—, *—(CH₂)₄—O—CO—, *—(CH₂)₆—O—CO— and *—(CH₂)₈—O—CO—. Examples of *-L^{b7}-O-L^{b6}- include *—CH₂—O—CH₂—. Examples of *—CO—O-L^{b5}-O— include *—CO—O—CH₂—O—, *—CO—O—(CH₂)₂—O—, *—CO—O—(CH₂)₃—O—, *—CO—O—(CH₂)₄—O— and *—CO—O—(CH₂)₆—O—. Examples of *—CO—O-L^{b10}-O-L^{b5}-CO—O— include the followings.

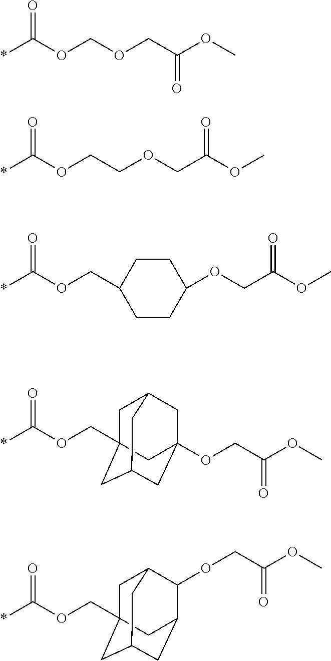

Examples of the substituent in Y include a halogen atom, a hydroxyl group, an oxo group, a glycidyloxy group, a C2-C4 acyl group, a C1-C12 alkoxy group, a C2-C7 alkoxycarbonyl group, a C1-C12 aliphatic hydrocarbon group, a C1-C12 hydroxy-containing aliphatic hydrocarbon group, a C3-C16 saturated cyclic hydrocarbon group, a C6-C18 aromatic hydrocarbon group, a C7-C21 aralkyl group and —(CH₂)ⱼ₂—O—CO—R^{b1}— in which R^{b1} represents a C1-C16 aliphatic hydrocarbon group, a C3-C16 saturated cyclic hydrocarbon group or a C6-C18 aromatic hydrocarbon group and j2 represents an integer of 0 to 4. Examples of the halogen atom include a fluorine atom, a chlorine atom, a bromine atom and an iodine atom. Examples of the acyl group include an acetyl group and a propionyl group, and examples of the alkoxy group include a methoxy group, an ethoxy group, a propoxy group, an isopropoxy group and a butoxy group. Examples of the alkoxycarbonyl group include a methoxycarbonyl group, an ethoxycarbonyl group, a propoxycarbonyl group, an isopropoxycarbonyl group and a butoxycarbonyl group. Examples of the aliphatic hydrocarbon group include the same as described above. Examples of the hydroxyl-containing aliphatic hydrocarbon group include a hydroxymethyl group. Examples of the C3-C16 saturated cyclic hydrocarbon group include the same as described above, and examples of the aromatic hydrocarbon group include a phenyl group, a naphthyl group, an anthryl group, a p-methylphenyl group, a p-tert-butylphenyl group and a p-adamantylphenyl group. Examples of the aralkyl group include a benzyl group, a phenethyl group, a phenylpropyl group, a trityl group, a naphthylmethyl group and a naphthylethyl group.

Examples of the C1-C18 aliphatic hydrocarbon group represented by Y include a methyl group, an ethyl group, a propyl group, an isopropyl group, a butyl group, an isobutyl group, a sec-butyl group, a tert-butyl group, a pentyl group, a neopentyl group, a 1-methylbutyl group, a 2-methylbutyl group, a 1,2-dimethylpropyl group, a 1-ethylpropyl group, a hexyl group, a 1-methylpentyl group, aheptyl group, anoctyl group, a 2-ethylhexyl group, a nonyl group, a decyl group, an undecyl group and a dodecyl group, and a C1-C6 alkyl group is preferable. Examples of the C3-C36 saturated cyclic hydrocarbon group represented by Y include the groups represented by the formulae (Y1) to (Y26):

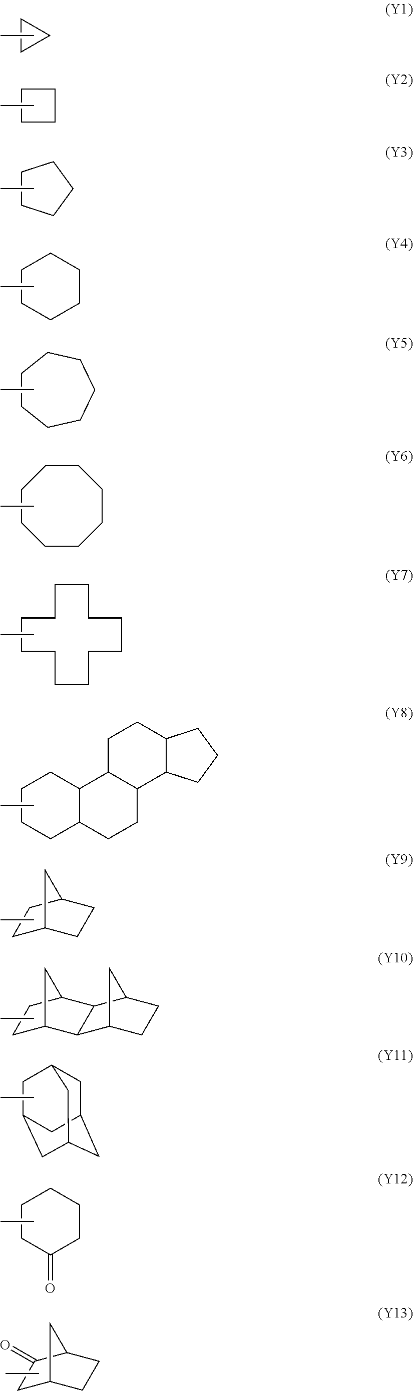

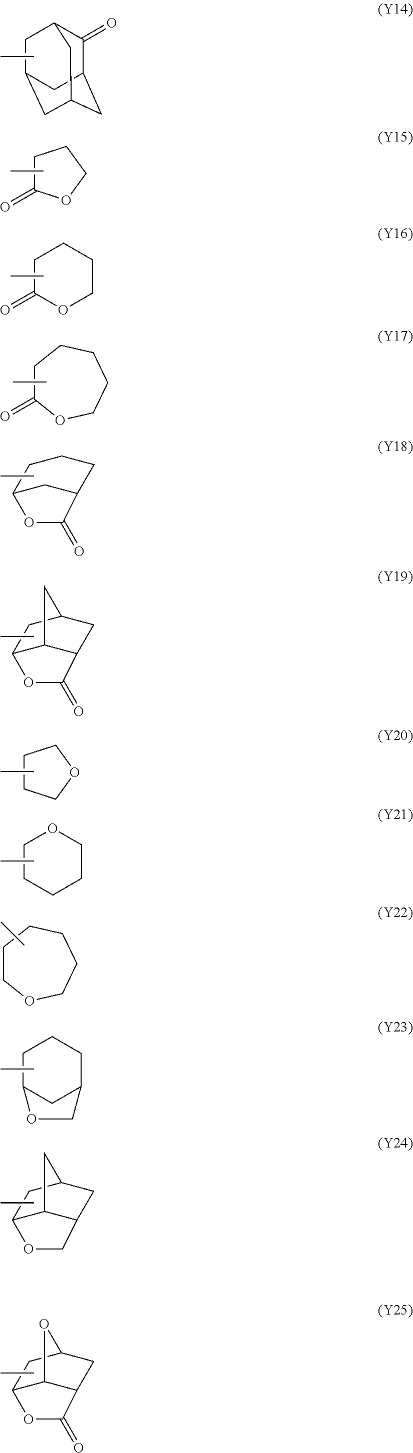

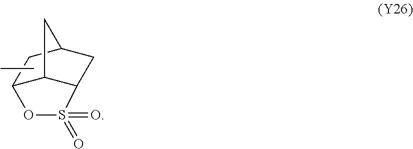

Among them, preferred are the groups represented by the formulae (Y1) to (Y19), and more preferred are the groups represented by the formulae (Y11), (Y14), (Y15) and (Y19). The groups represented by the formulae (Y11) and (Y14) are especially preferable.

Examples of Y having one or more substituents include the followings:

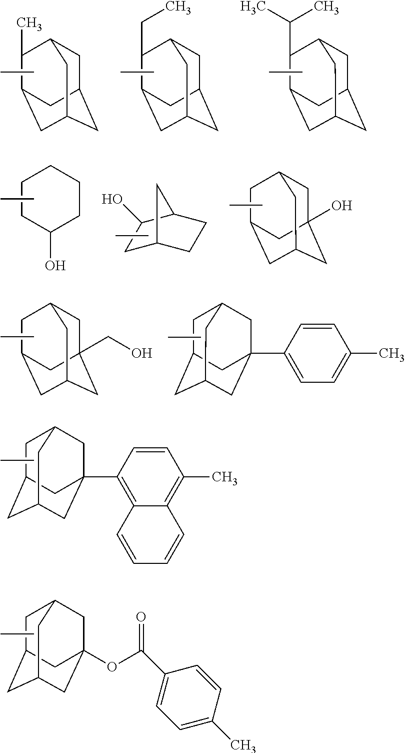

Y is preferably an adamantyl group which can have one or more substituents, and is more preferably an adamantyl group or an oxoadamantyl group.

Among the sulfonic acid anions of the acid generator represented by the formula (B1), preferred is a sulfonic acid anion in which L^{b1} is *—CO—O-L^{b2}-, and more preferred are anions represented by the formulae (b1-1-1) to (b1-1-9).

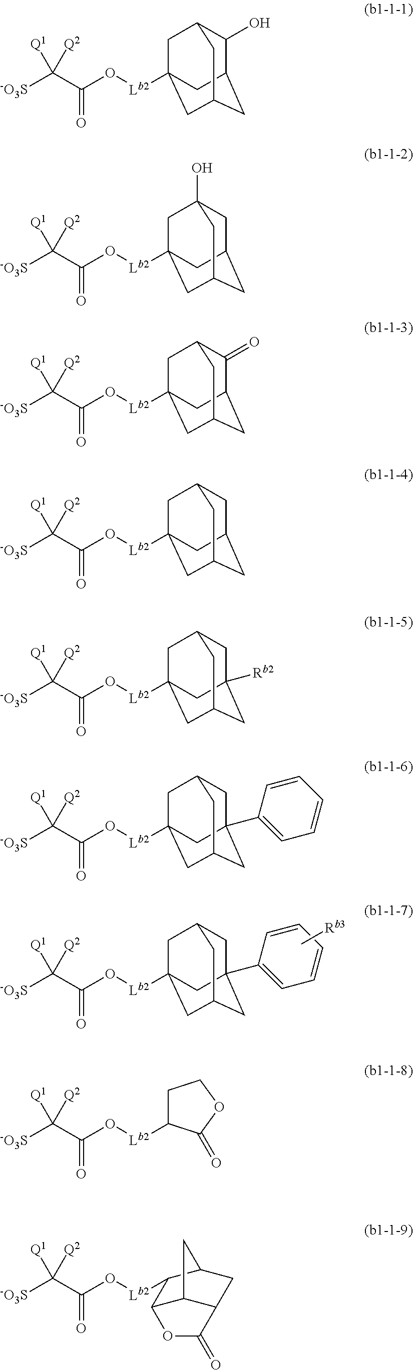

wherein Q¹, Q² and L^{b2} are the same as defined above, and R^{b2} and R^{b3} each independently represent a C1-C4 aliphatic hydrocarbon group, preferably a methyl group.

Examples of the anions of the acid generator represented by the formula (B1) include the followings.

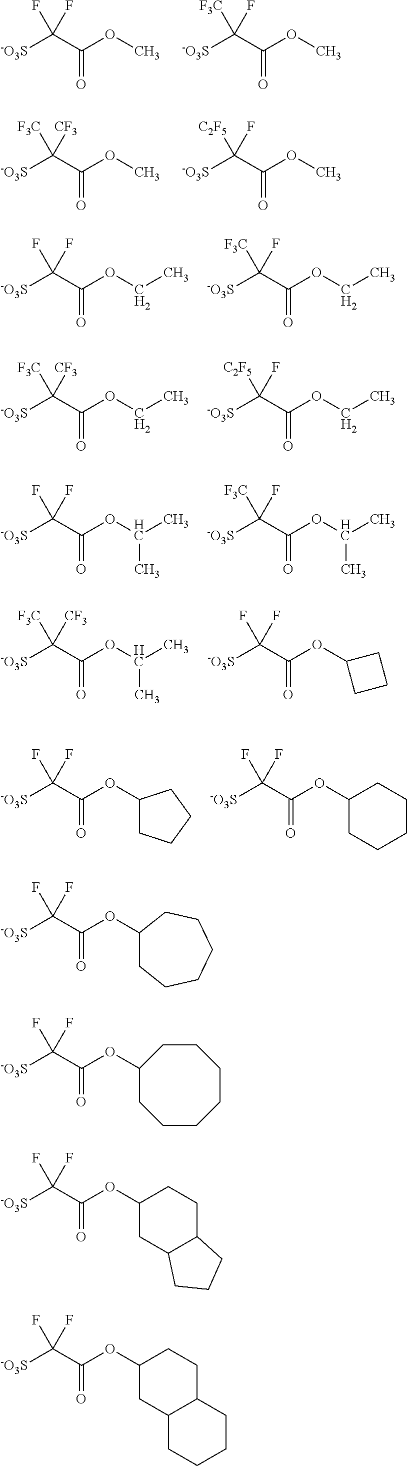

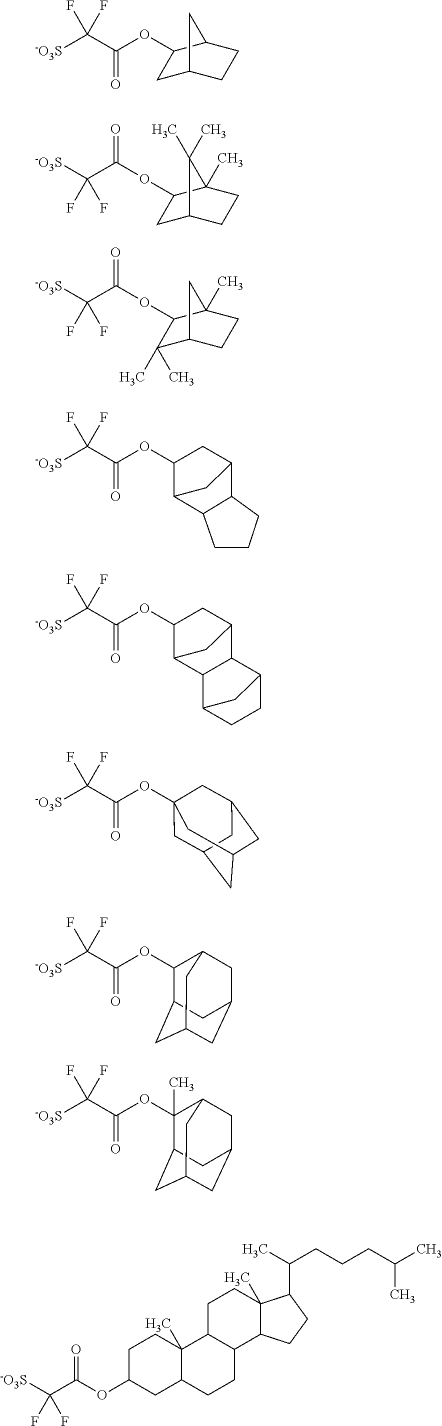

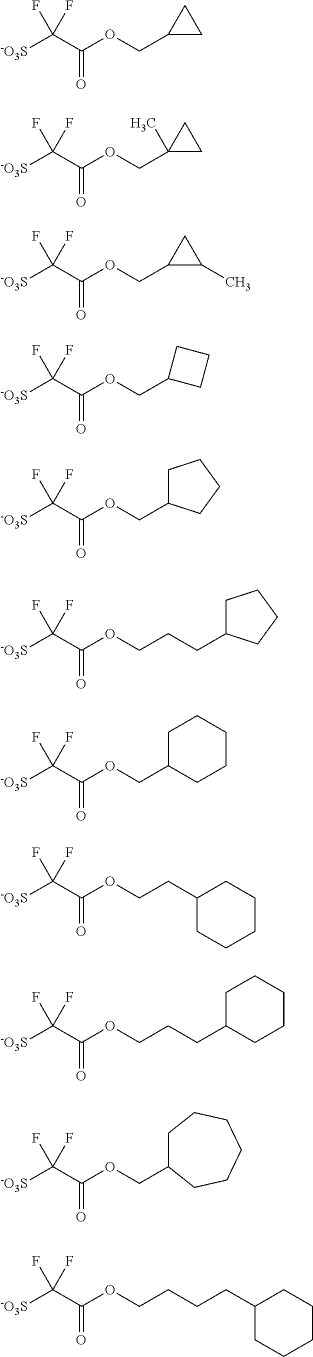

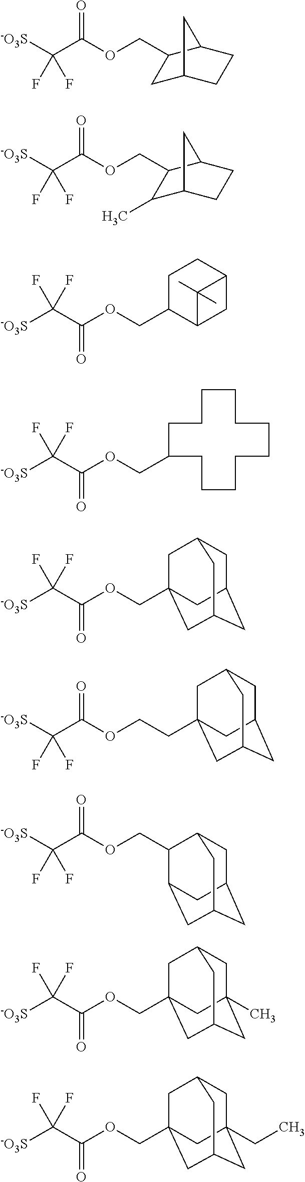

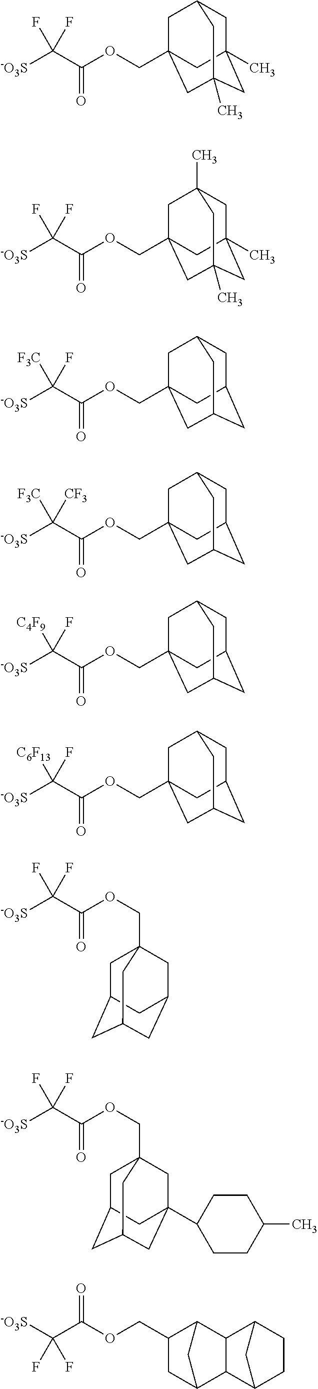

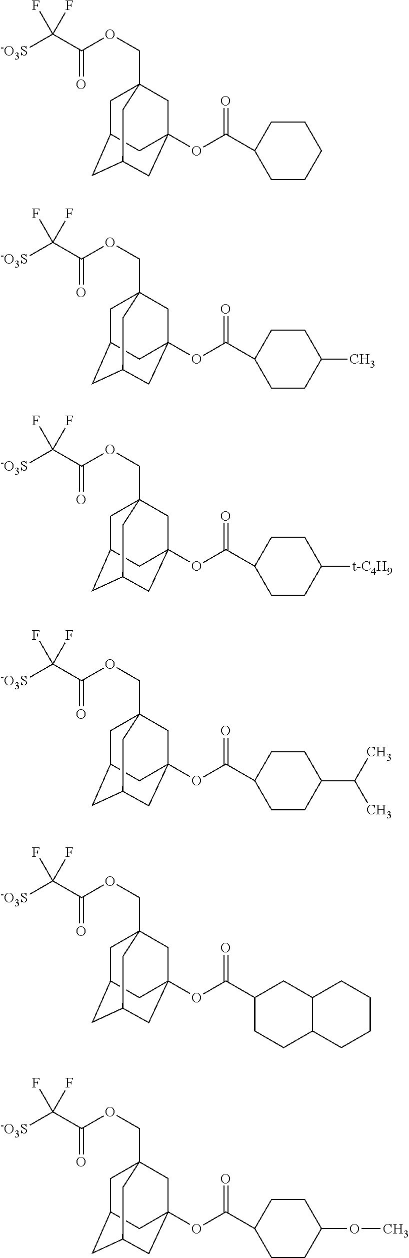

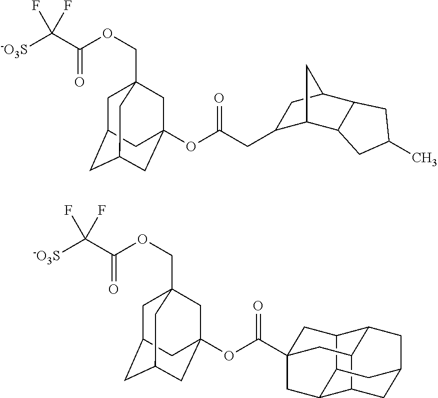

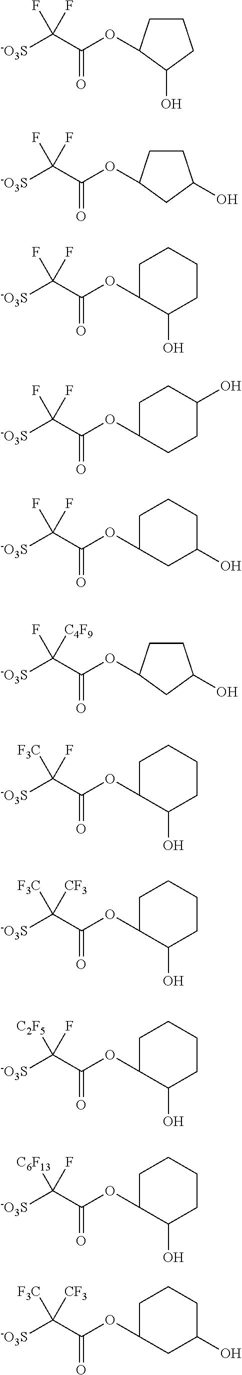

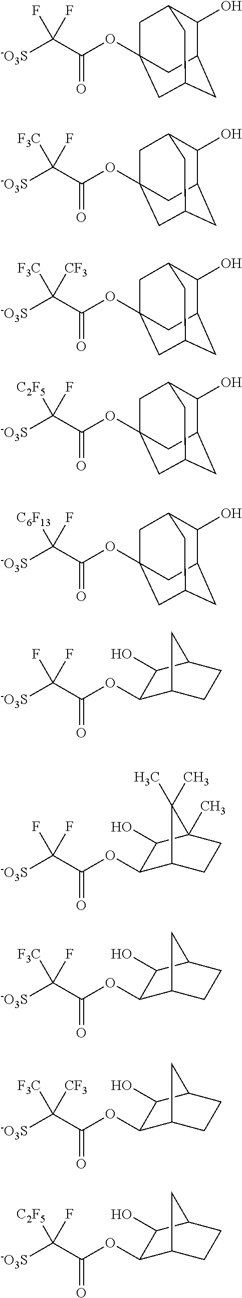

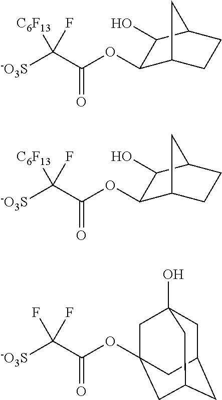

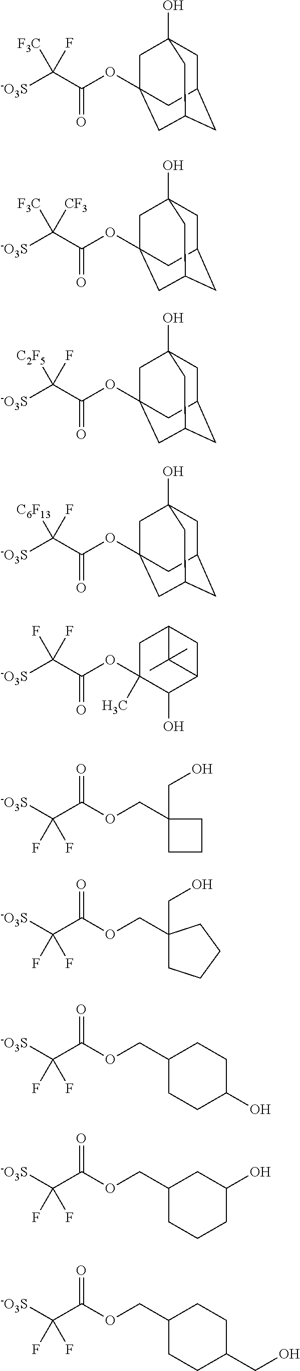

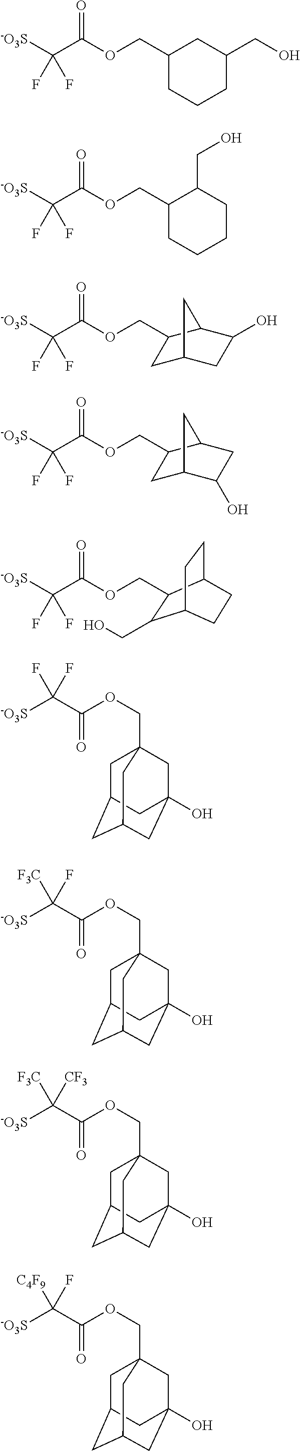

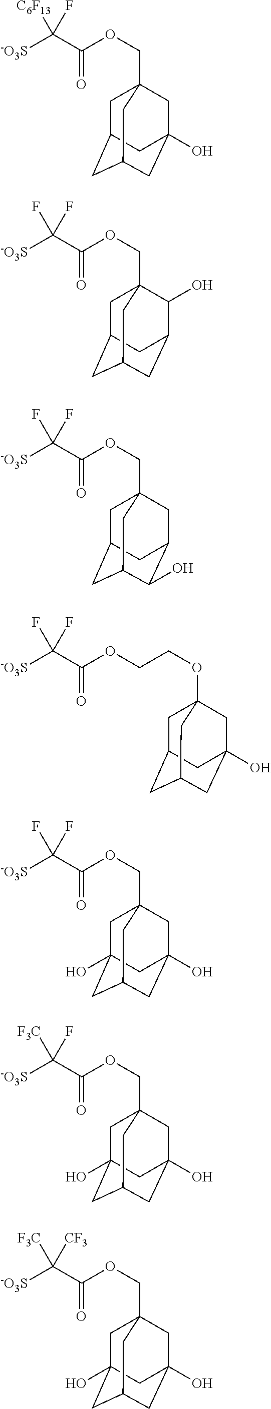

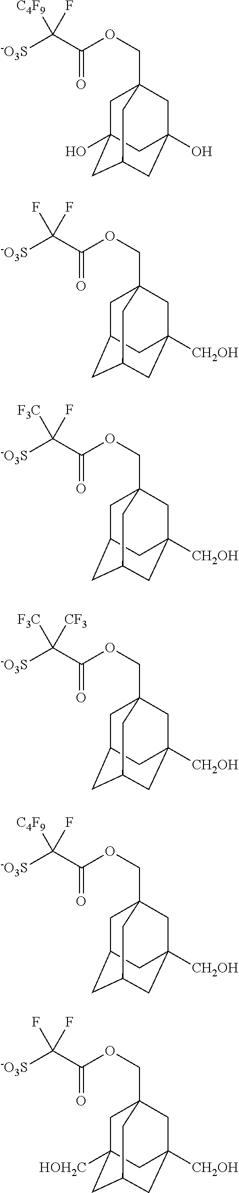

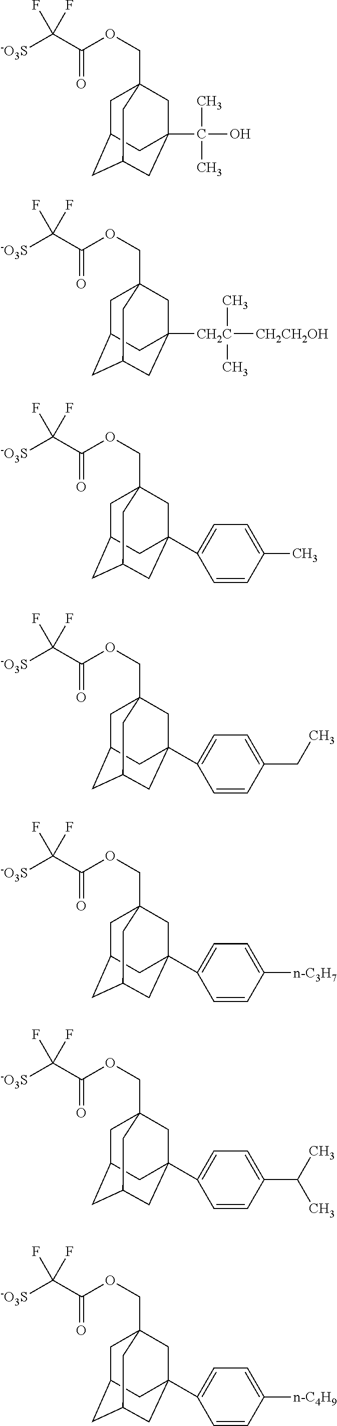

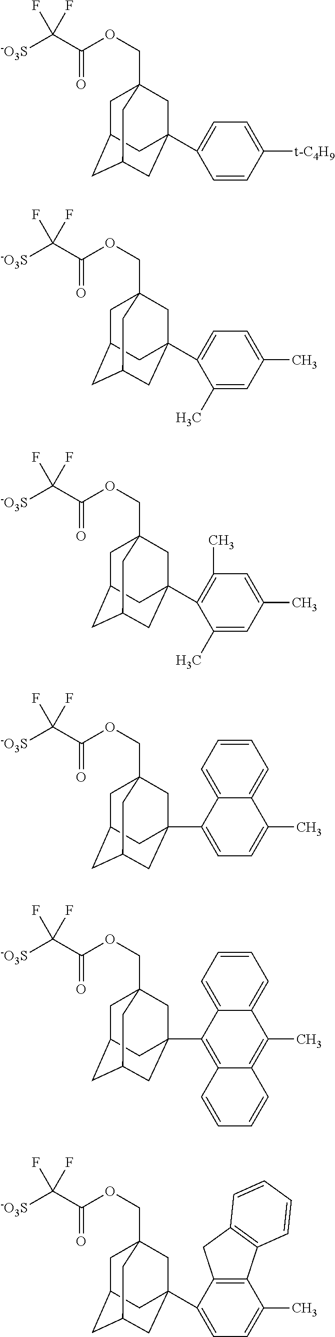

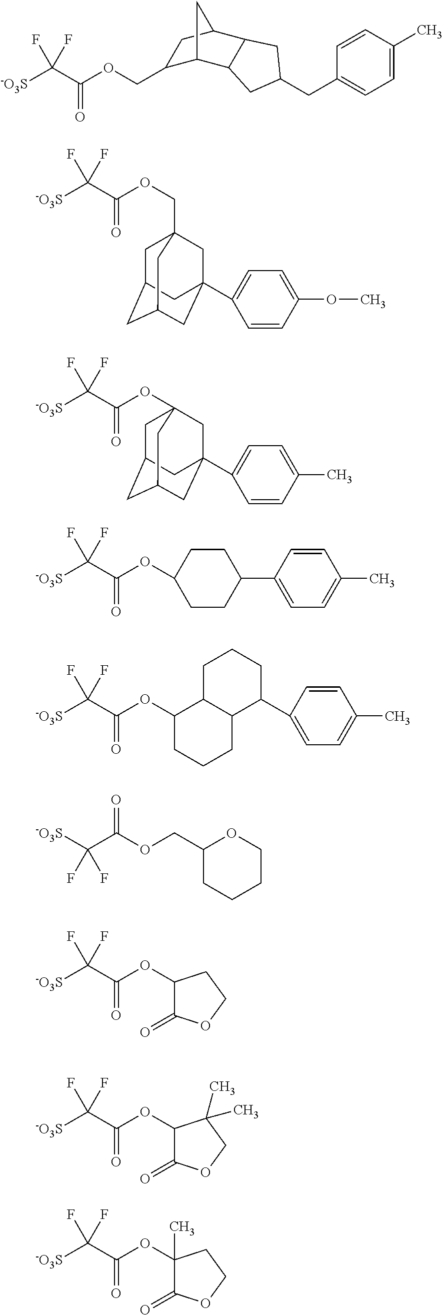

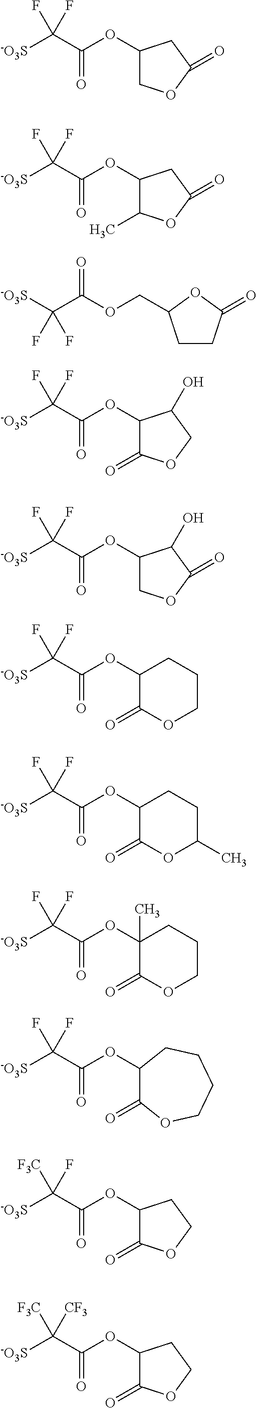

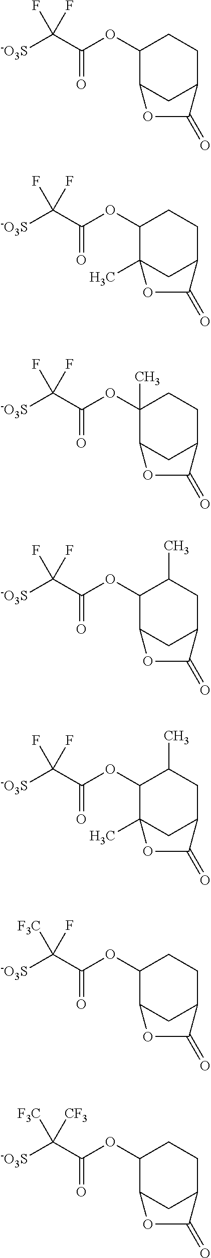

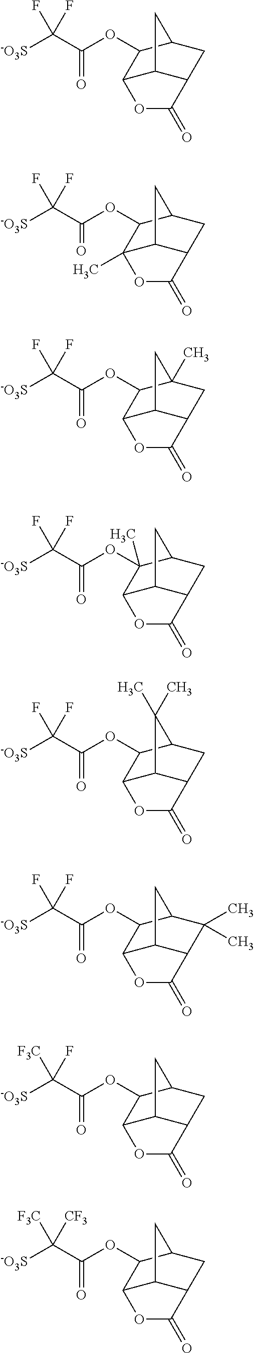

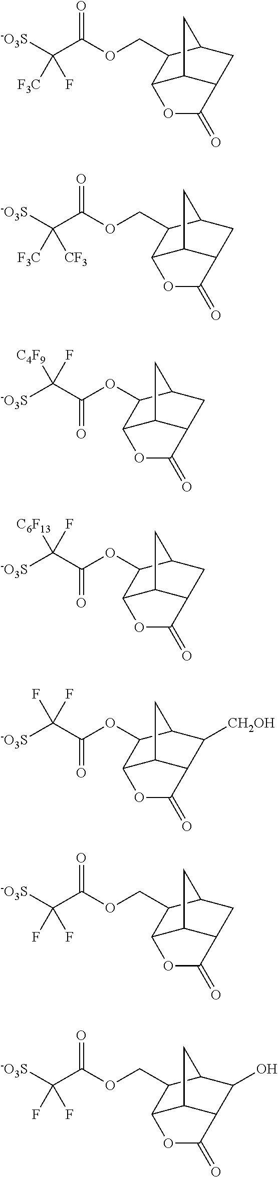

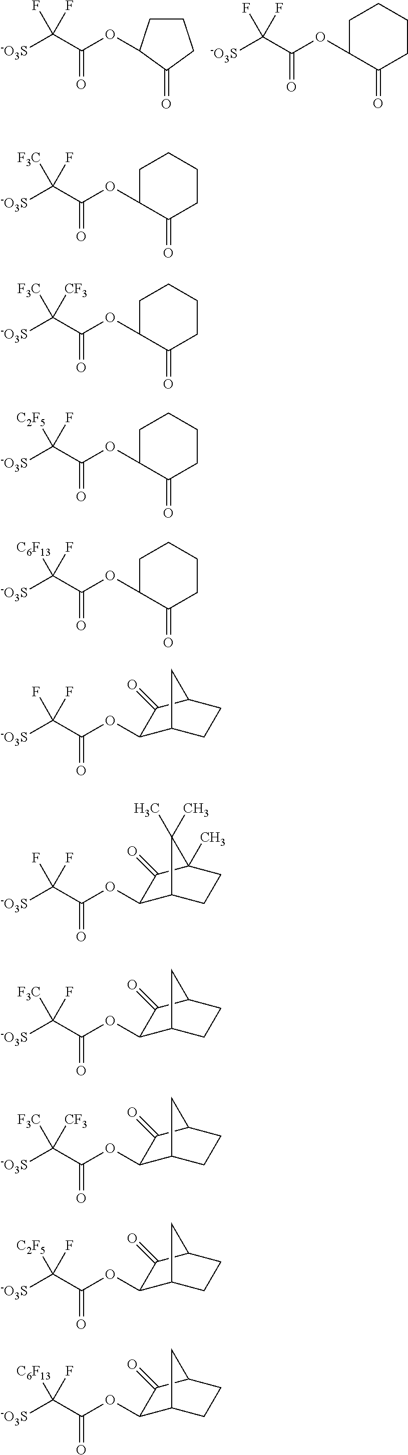

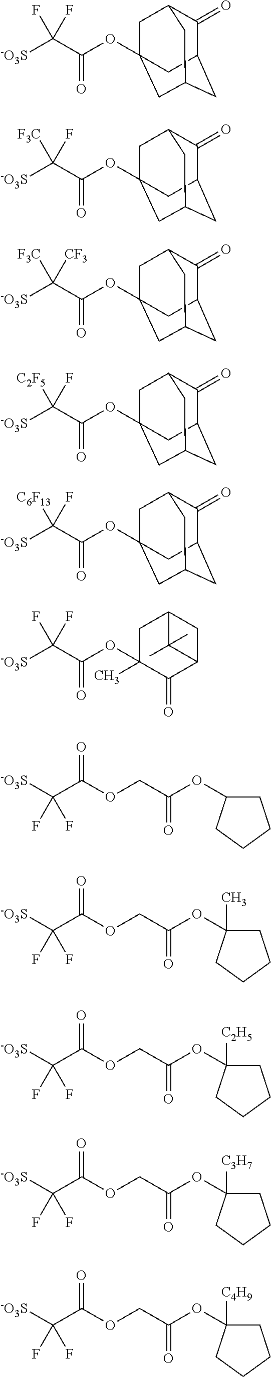

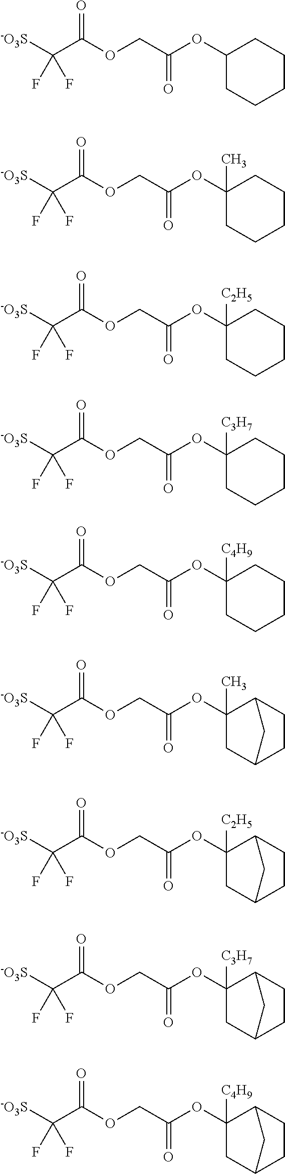

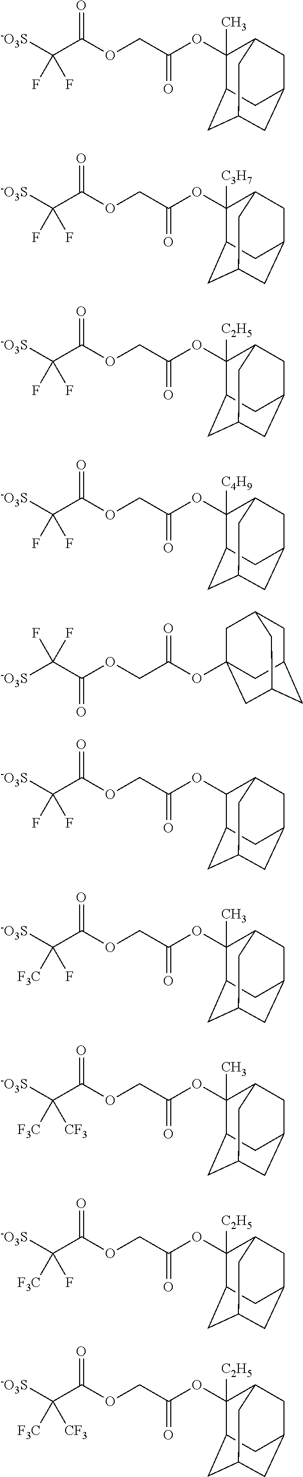

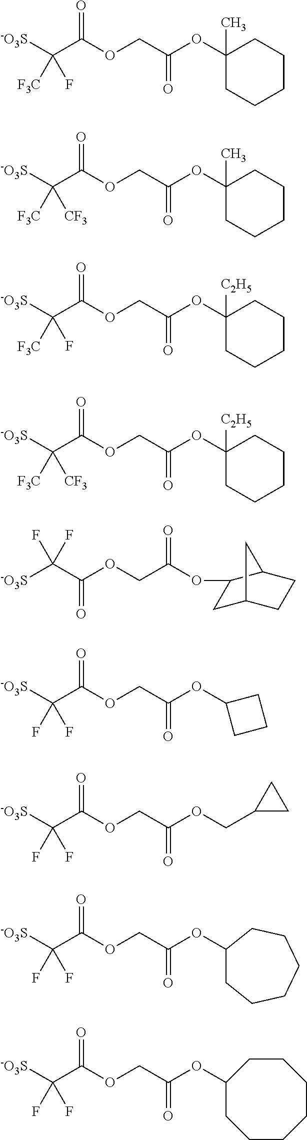

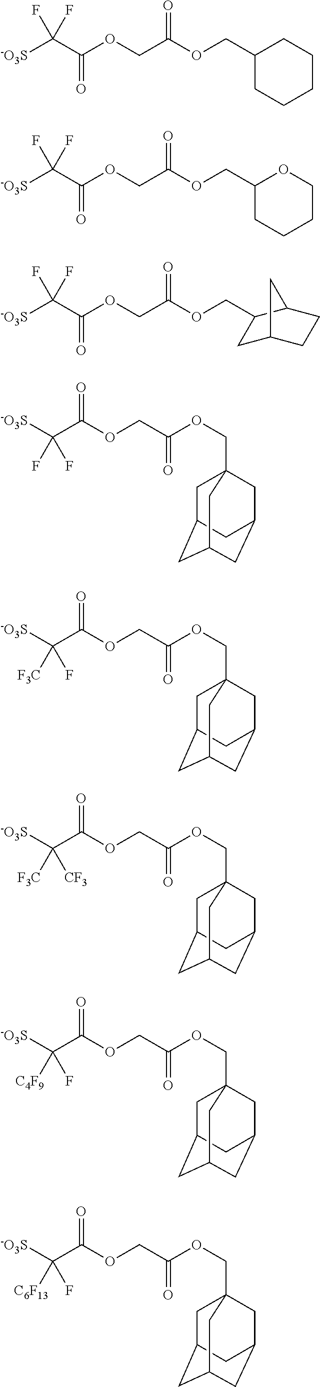

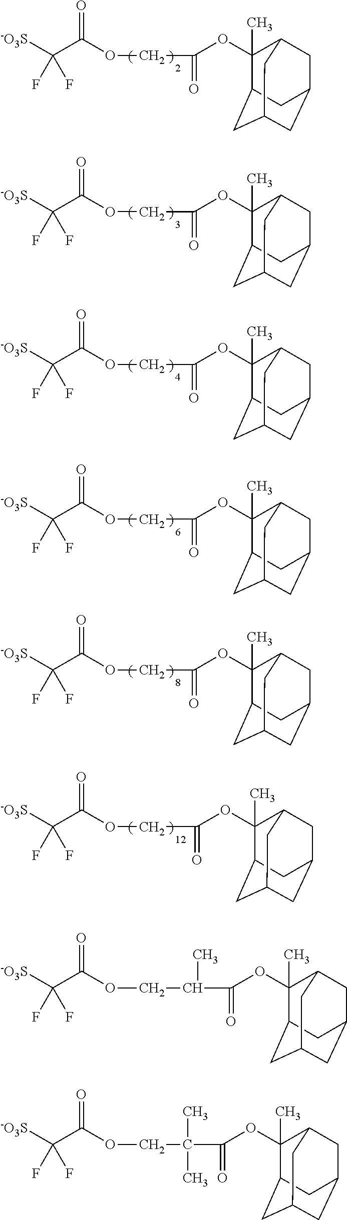

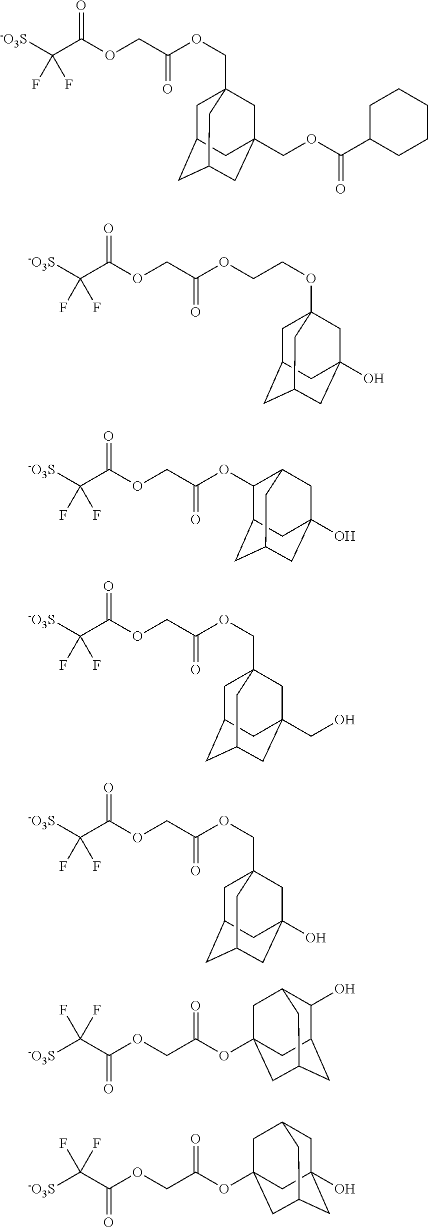

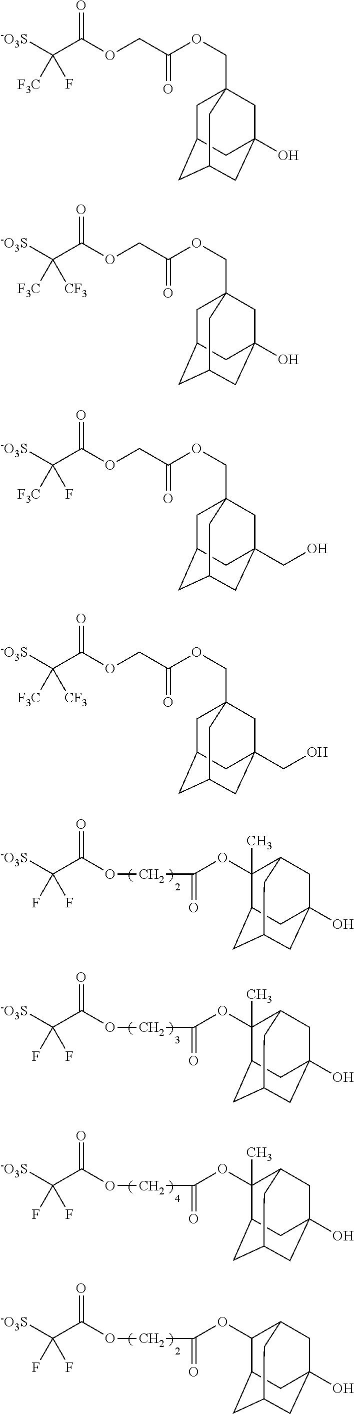

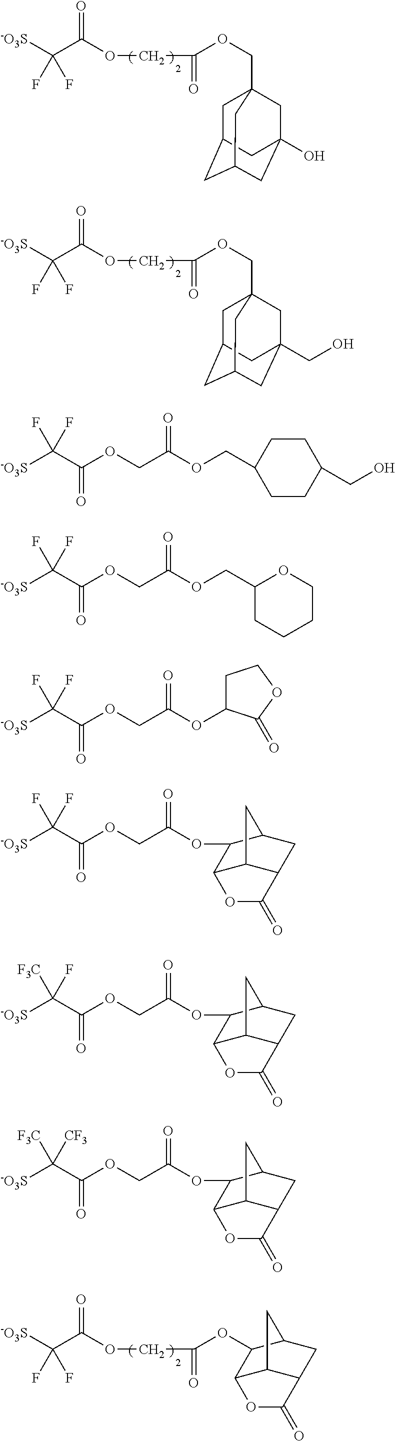

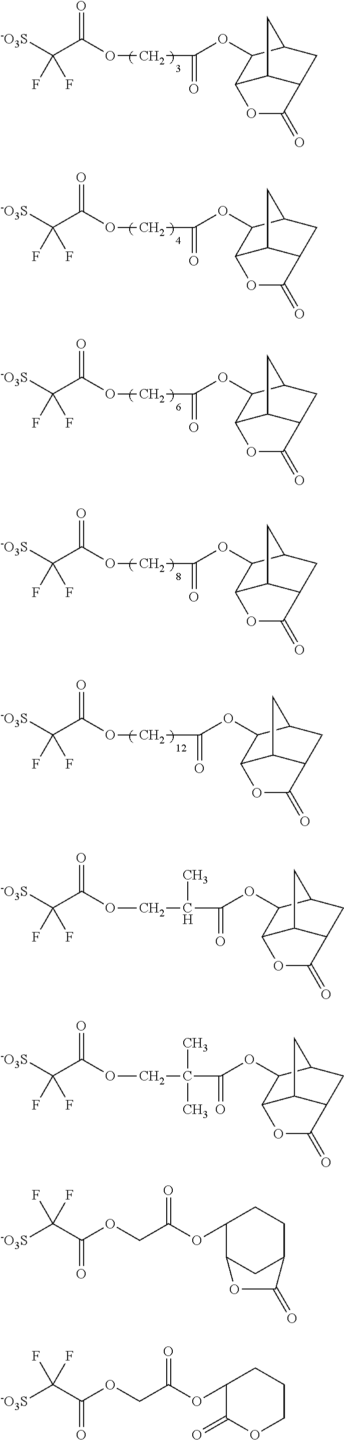

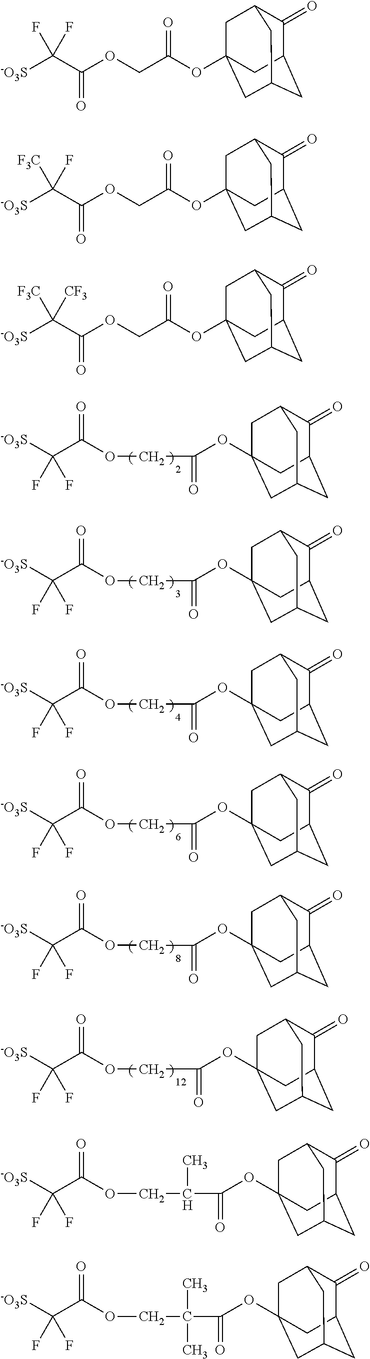

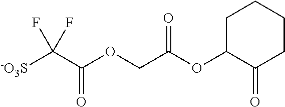

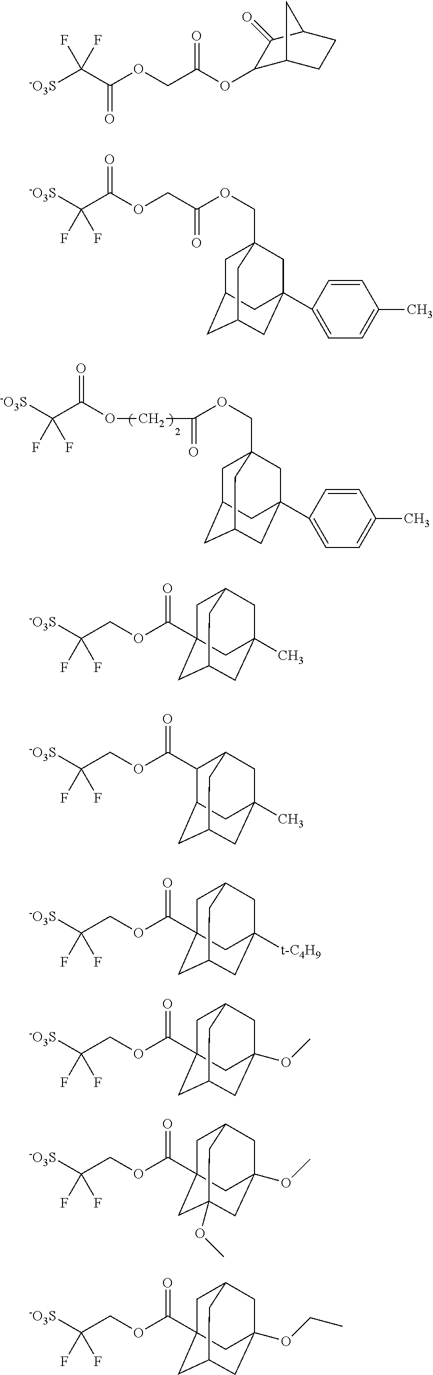

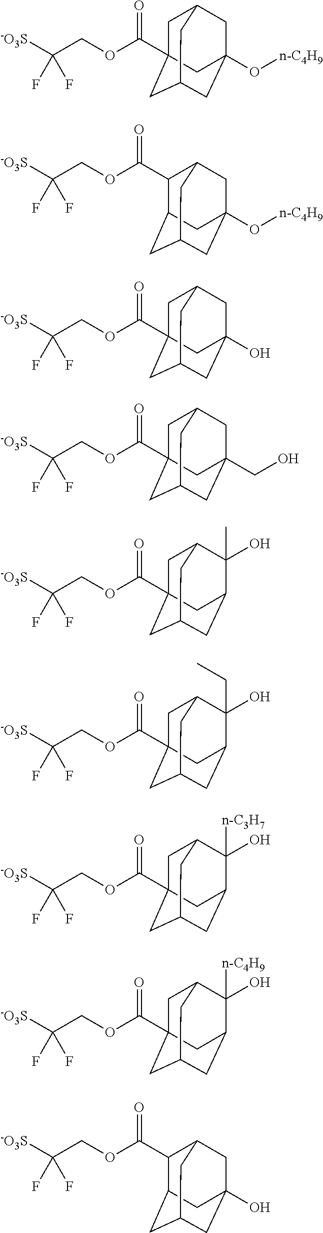

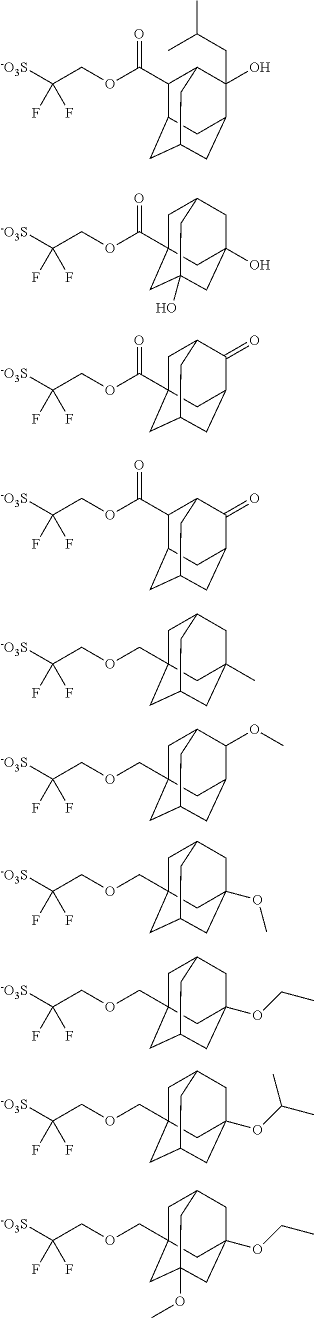

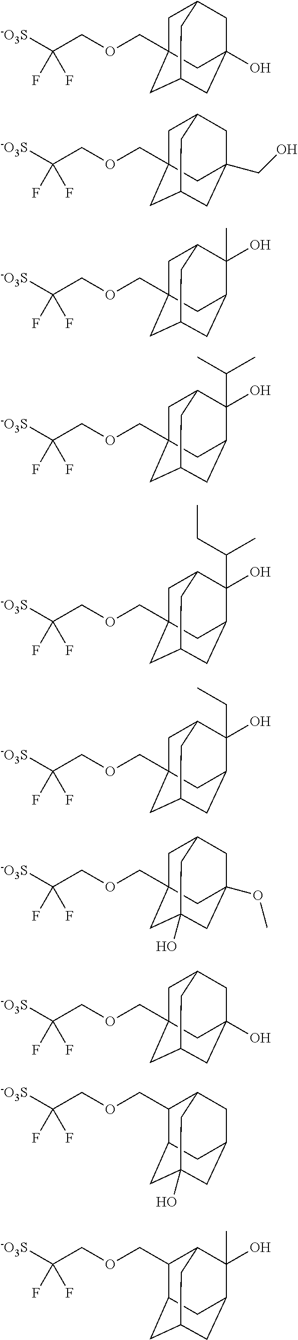

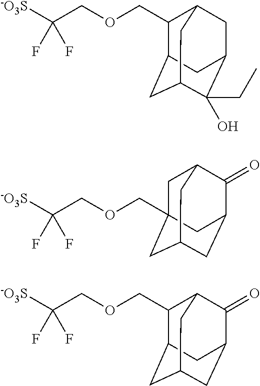

Among them, preferred are the following sulfonic anions.

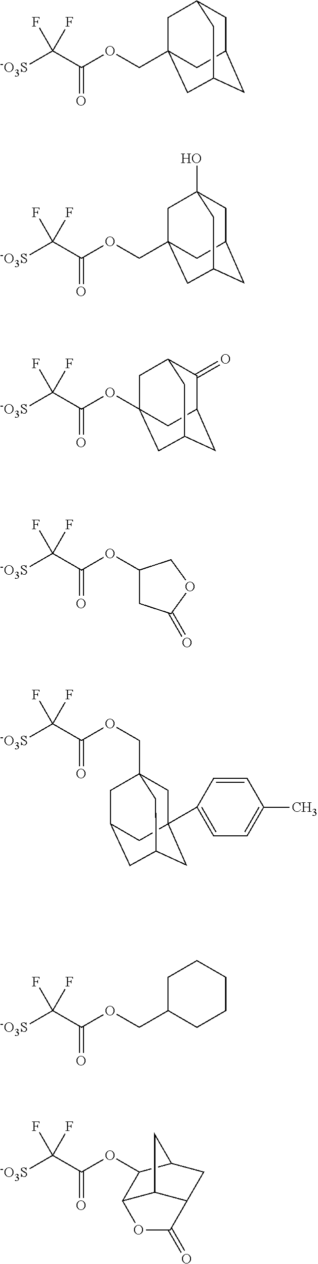

Examples of the organic counter ion represented by Z⁺ in the acid generator represented by the formula (B1) include an onium cation such as a sulfonium cation, an iodonium cation, an ammonium cation, a benzothiazolium cation and a phosphonium cation, and a sulfonium cation and an iodonium cation are preferable, and an arylsulfonium cation is more preferable, and triarylsulfonium cation is especially, preferable.

Preferable examples of the organic cation represented by Z¹⁺ include the cations represented by the formulae (b2-1) to (b2-4):

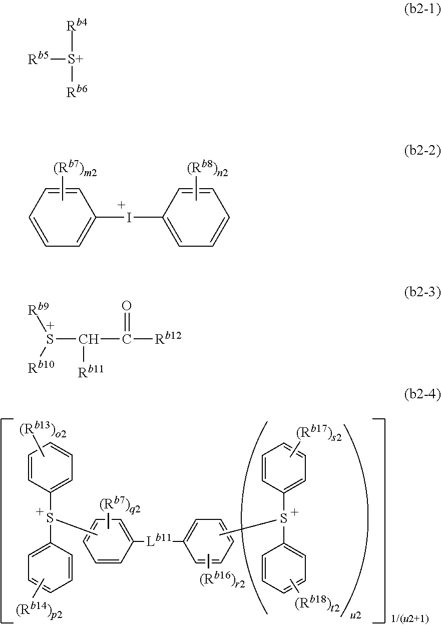

wherein R^{b4}, R^{b5} and R^{b6} independently represent a C1-C30 aliphatic hydrocarbon group which can have one or more substituents selected from the group consisting of a hydroxyl group, a C1-C12 alkoxy group and a C6-C18 aromatic hydrocarbon group, a C3-C18 saturated cyclic hydrocarbon group which can have one or more substituents selected from the group consisting of a halogen atom, a C2-C4 acyl group and a glycidyloxy group, or a C6-C18 aromatic hydrocarbon group which can have one or more substituents selected from the group consisting of a halogen atom, a hydroxyl group, a C1-C18 aliphatic hydrocarbon group, a C3-C18 saturated cyclic hydrocarbon group and a C1-C12 alkoxy group,
R^{b7} and R^{b8} are independently in each occurrence a hydroxyl group, a C1-C12 aliphatic hydrocarbon group or a C1-C12 alkoxy group, m2 and n2 independently represents an integer of 0 to 5,
R^{b9} and R^{b10} independently represent a C1-C18 aliphatic hydrocarbon group or a C3-C18 saturated cyclic hydrocarbon group, or R^{b9} and R^{b10} are bonded to form a C2-C11 divalent acyclic hydrocarbon group which forms a ring together with the adjacent S⁺, and one or more —CH₂— in the divalent acyclic hydrocarbon group may be replaced by —CO—, —O— or —S—,
and
R^{b11} represents a hydrogen atom, a C1-C18 aliphatic hydrocarbon group, a C3-C18 saturated cyclic hydrocarbon group or a C6-C18 aromatic hydrocarbon group, R^{b12} represents a C1-C12 aliphatic hydrocarbon group, a C3-C18 saturated cyclic hydrocarbon group or a C6-C18 aromatic hydrocarbon group and the aromatic hydrocarbon group can have one or more substituents selected from the group consisting of a C1-C12 aliphatic hydrocarbon group, a C1-C12 alkoxy group, a C3-C18 saturated cyclic hydrocarbon group and a C2-C13 acyloxy group, or R^{b11} and R^{b12} are bonded each other to form a C1-C10 divalent acyclic hydrocarbon group which forms a 2-oxocycloalkyl group together with the adjacent —CHCO—, and one or more —CH₂— in the divalent acyclic hydrocarbon group may be replaced by —CO—, —O— or —S—, and
R^{b13}, R^{b14}, R^{b15}, R^{b16}, R^{b17} and R^{b18} independently represent a hydroxyl group, a C1-C12 aliphatic hydrocarbon group or a C1-C12 alkoxy group, L^{b11} represents —S— or —O— and o2, p2, s2 and t2 each independently represents an integer of 0 to 5, q2 and r2 each independently represents an integer of 0 to 4, and u2 represents 0 or 1.

The aliphatic hydrocarbon group represented by R^{b9} to R^{b11} has preferably 1 to 12 carbon atoms. The saturated cyclic hydrocarbon group represented by R^{b9} to R^{b11} has preferably 3 to 18 carbon atoms and more preferably 4 to 12 carbon atoms.

Preferable examples of the aliphatic hydrocarbon group include an alkyl group such as a methyl group, an ethyl group, a propyl group, an isopropyl group, a butyl group, a sec-butyl group, a tert-butyl group, a pentyl group, a hexyl group, an octyl group and a 2-ethylhexyl group. Preferable examples of the saturated cyclic hydrocarbon group include a cyclopropyl group, a cyclobutyl group, a cyclopentyl group, a cyclohexyl group, a cycloheptyl group, a cyclodecyl group, a 2-alkyl-a-adamantyl group, a 1-(1-adamantyl)-1-alkyl group and an isobornyl group. Preferable examples of the aromatic group include a phenyl group, a 4-methylphenyl group, a 4-ethylphenyl group, a 4-tert-butylphenyl group, a 4-cyclohexylphenyl group, a 4-methoxyphenyl group, a biphenyl group and a naphthyl group. Examples of the aliphatic hydrocarbon group having an aromatic hydrocarbon group include a benzyl group. Examples of the alkoxy group include a methoxy group, an ethoxy group, a propoxy group, an isopropoxy group, a butoxy group, a sec-butoxy group, a tert-butoxy group, a pentyloxy group, a hexyloxy group, a heptyloxy group, an octyloxy group, a 2-ethylhexyloxy group, a nonyloxy group, a decyloxy group, an undecyloxy group and a dodecyloxy group.

Examples of the C3-C12 divalent acyclic hydrocarbon group formed by bonding R^{b9} and R^{b10} include a trimethylene group, a tetramethylene group and a pentamethylene group. Examples of the ring group formed together with the adjacent and the divalent acyclic hydrocarbon group include a thiolan-1-ium ring (tetrahydrothiphenium ring), a thian-1-ium ring and a 1,4-oxathian-4-ium ring. A C3-C7 divalent acyclic hydrocarbon group is preferable.

Examples of the C1-C10 divalent acyclic hydrocarbon group formed by bonding R^{b11} and R^{b12} include a methylene group, an ethylene group, a trimethylene group, a tetramethylene group and a pentamethylene group and examples of the ring group include the followings.

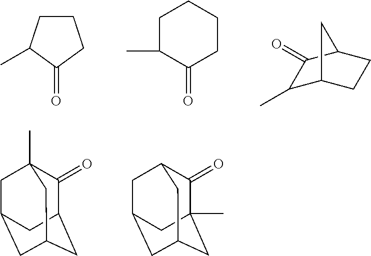

A C1-C5 divalent acyclic hydrocarbon group is preferable.

Among the above-mentioned cations, preferred is the cation represented by the formula (b2-1), and more preferred is the cation represented by the formula (b2-1-1). A triphenylsulfonium cation is especially preferable.

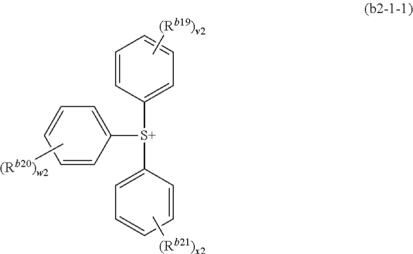

wherein R^{b19}, R^{b20} and R^{b21} are independently in each occurrence a halogen atom (preferably a fluorine atom), a hydroxyl group, a C1-C18 aliphatic hydrocarbon group, a C3-C18 saturated cyclic hydrocarbon group or a C1-C12 alkoxy group, and one or more hydrogen atoms of the aliphatic hydrocarbon group can be replaced by a hydroxyl group, a C1-C12 alkoxy group or a C6-C18 aromatic hydrocarbon group, and one or more hydrogen atoms of the saturated cyclic hydrocarbon group can be replaced by a halogen atom, a glycidyloxy group or a C2-C4 acyl group, and v2, w2 and x2 independently each represent an integer of 0 to 5.

The aliphatic hydrocarbon group has preferably 1 to 12 carbon atoms, and the saturated cyclic hydrocarbon group has preferably 4 to 18 carbon atoms, and v2, w2 and x2 independently each preferably represent 0 or 1.

It is preferred that R^{b19}, R^{b20} and R^{b21} are independently in each occurrence a halogen atom, a hydroxyl group, a C1-C12 alkyl group or a C1-C12 alkoxy group, and v2, w2 and X² independently each represent an integer of 0 to 5. It is more preferred that R^{b19}, R^{b20} and R^{b21} are independently in each occurrence a fluorine atom, a hydroxyl group, a C1-C12 alkyl group or a C1-C12 alkoxy group, and v2, w2 and x2 independently each represent 0 or 1.

Examples of the cation represented by the formula (b2-1) include the following.

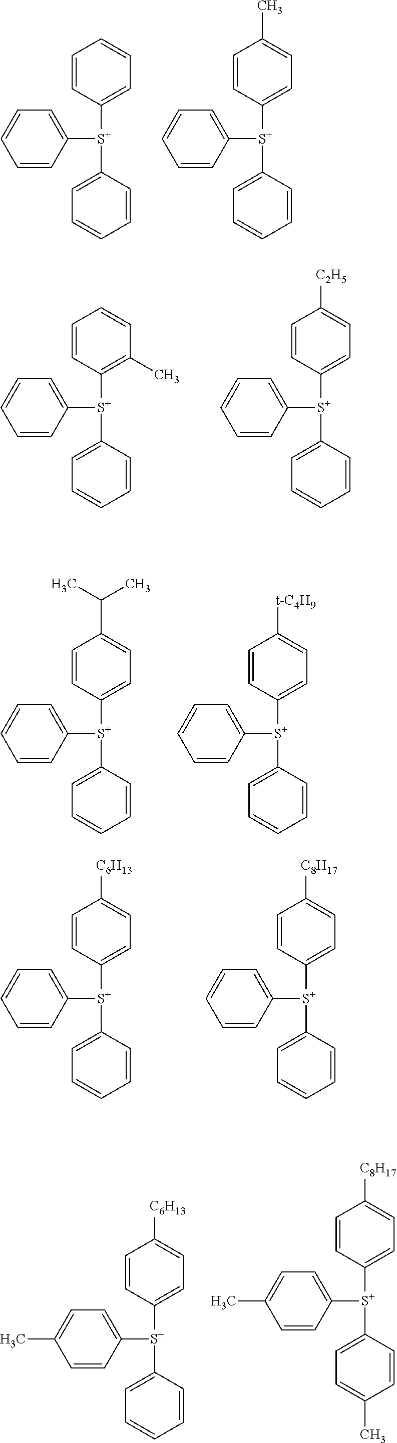

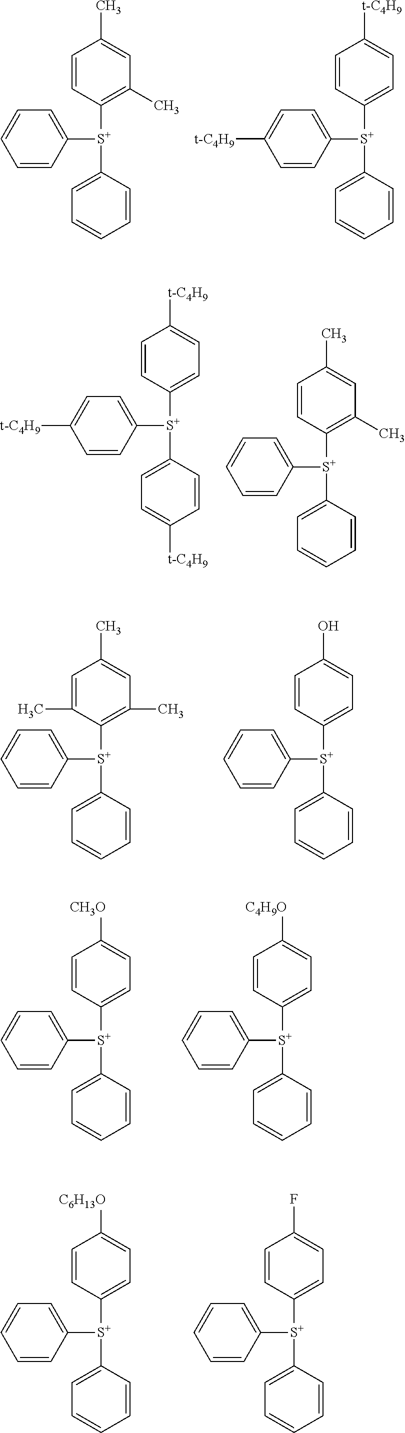

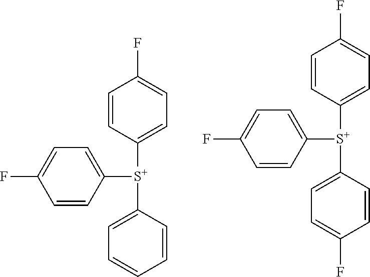

Examples of the cation represented by the formula (b2-2) include the followings.

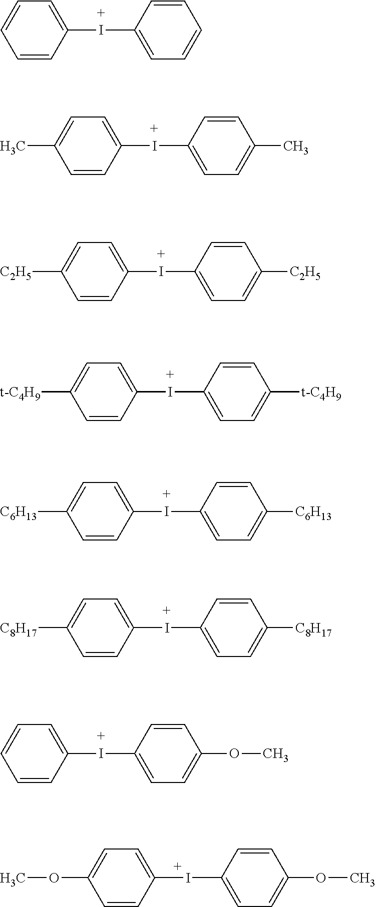

Examples of the cation represented by the formula (b2-3) include the followings.

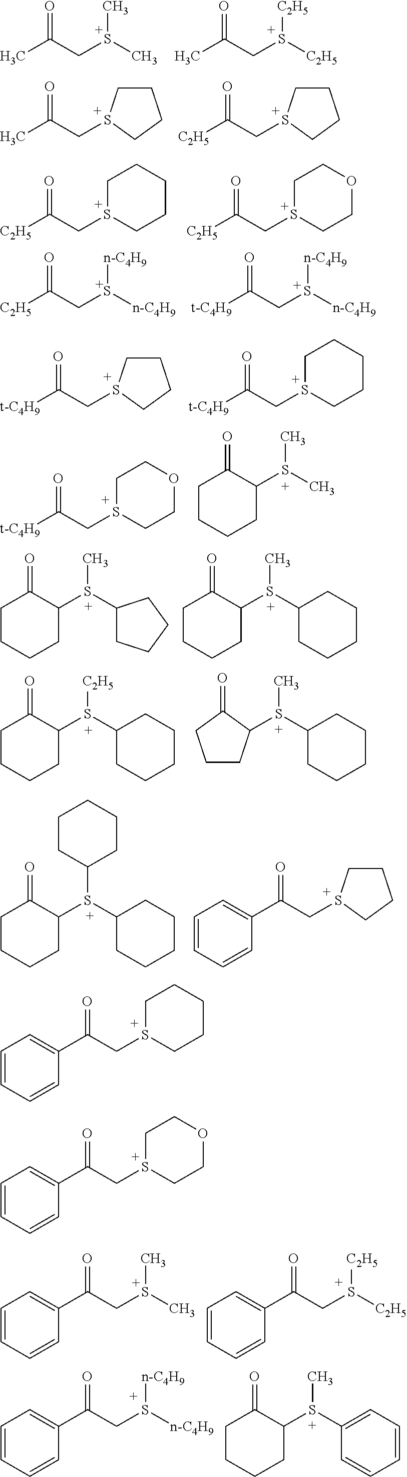

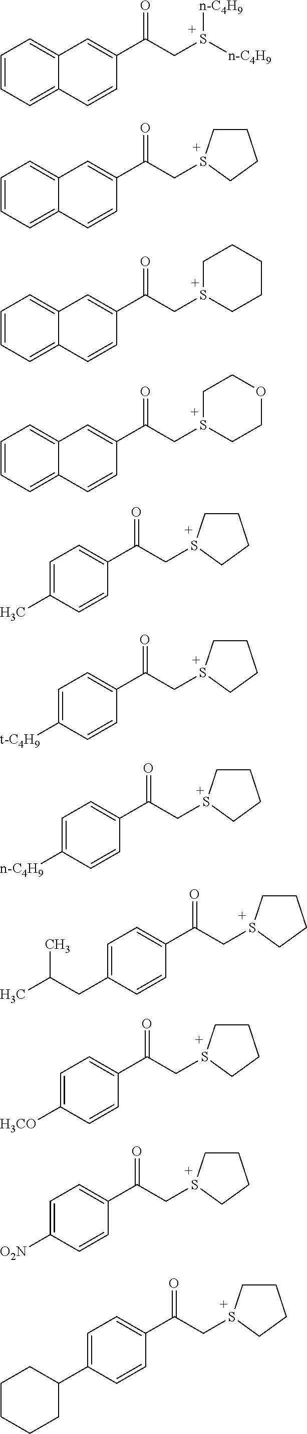

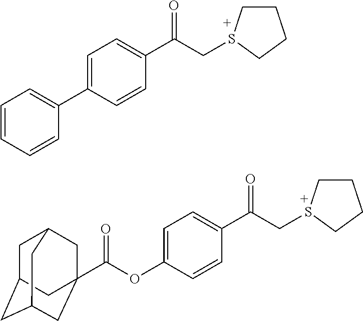

Examples of the cation represented by the formula (b2-4) include the followings.

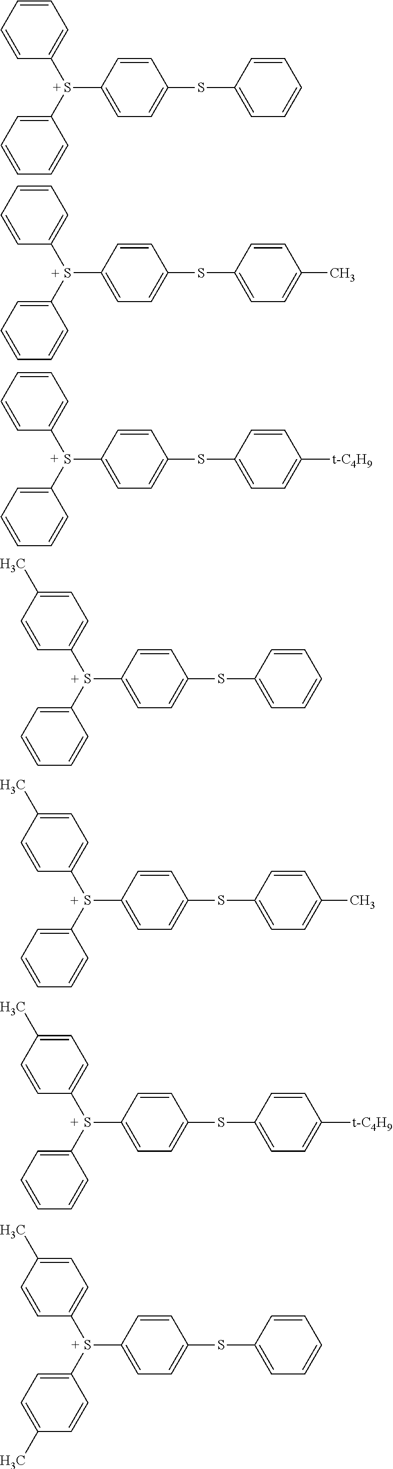

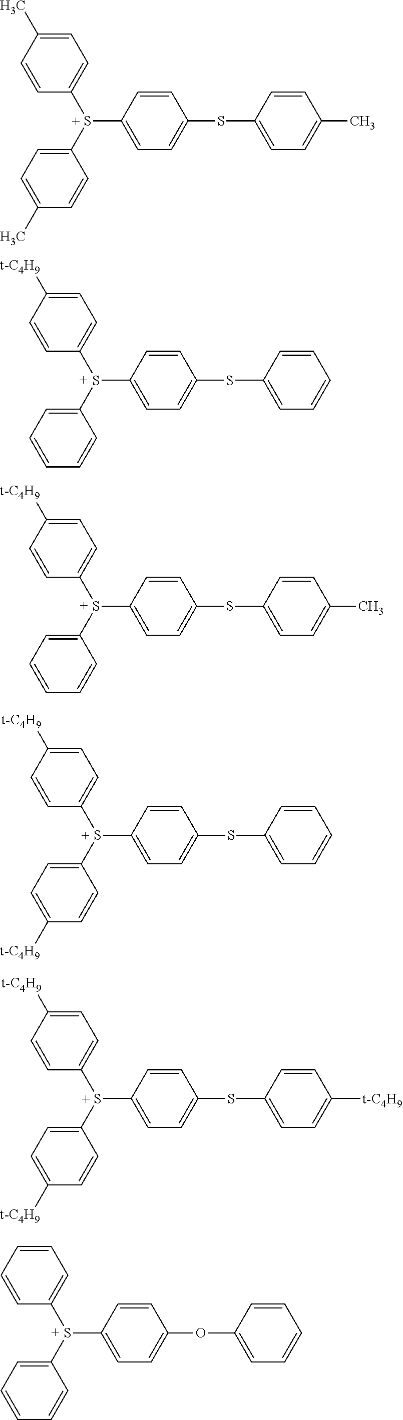

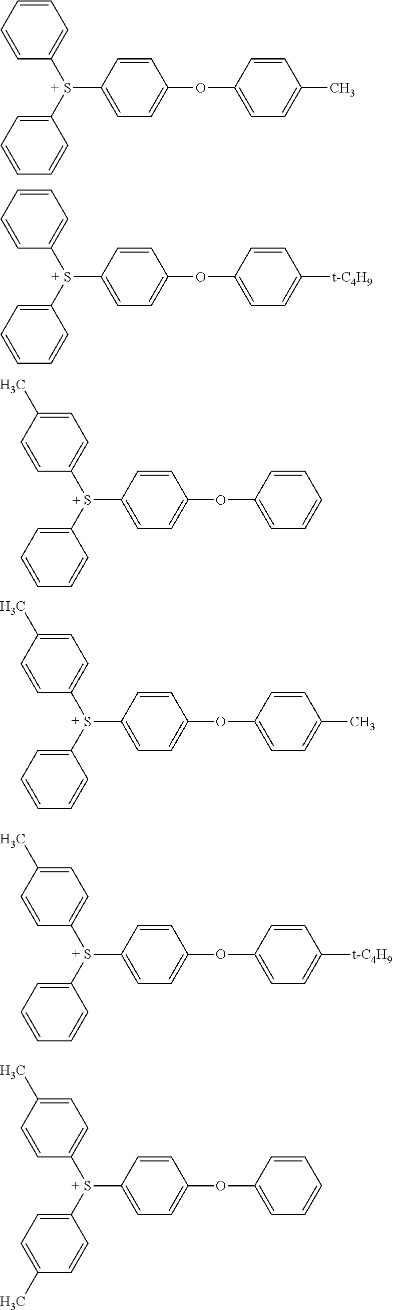

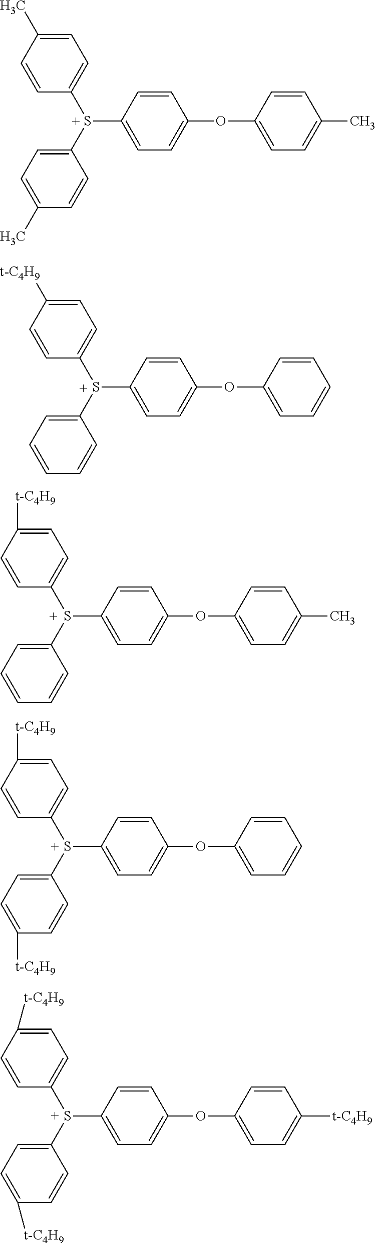

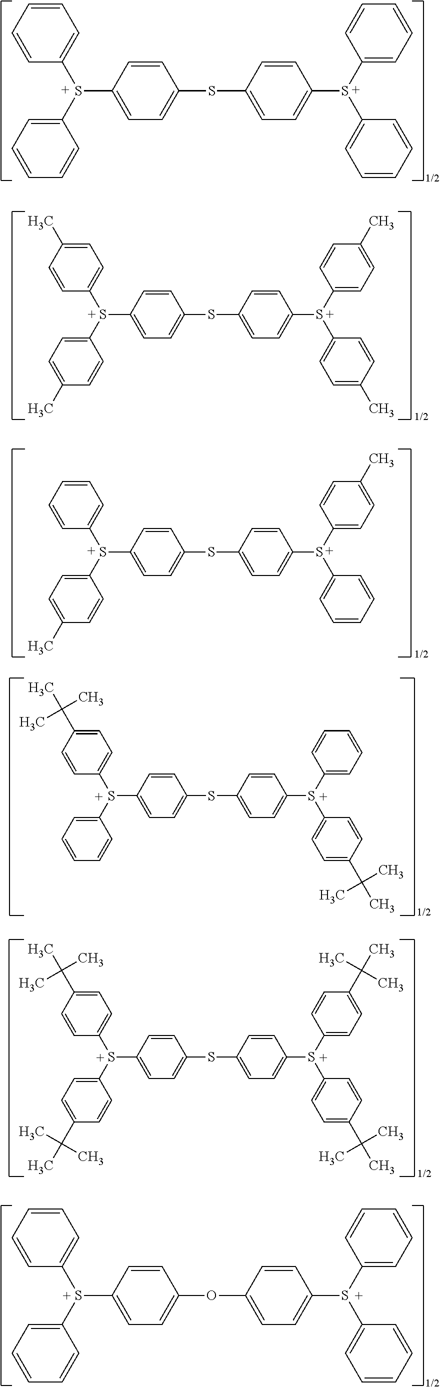

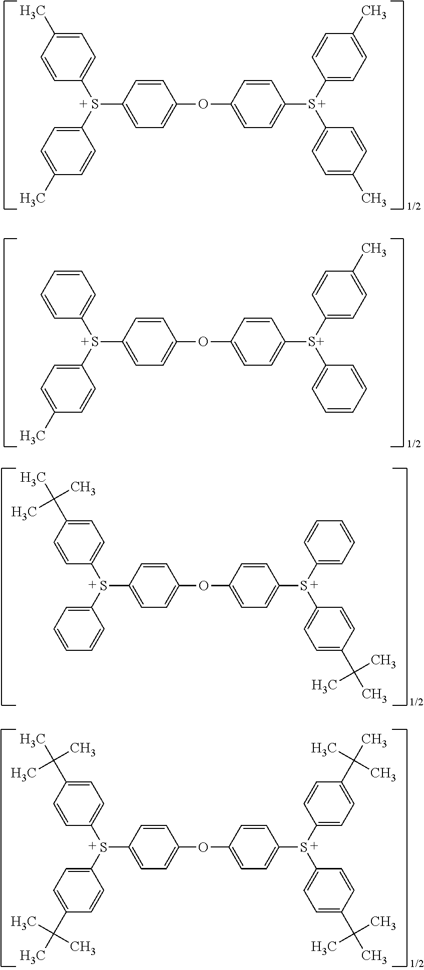

Examples of the acid generator represented by the formula (B1) include an acid generator wherein the anion is any one of the above-mentioned anion parts and the cation is any one of the above-mentioned cation parts. Preferable examples of the acid generator represented by the formula (B1) include a combination of anyone of anions represented by the formulae (b1-1-1) to (b1-1-9) and the cation represented by the formulae (b2-1-1), and a combination of any one of anions represented by the formulae (b1-1-3) to (b1-1-5) and the cation represented by the formulae (b2-3).

The acid generator represented by the formulae (B1-1) to (B1-17) are preferable, and the acid generator represented by the formulae (B1-1), (B1-2), (B1-6), (B1-11), (B1-12), (B1-13) and (B1-14) are more preferable.

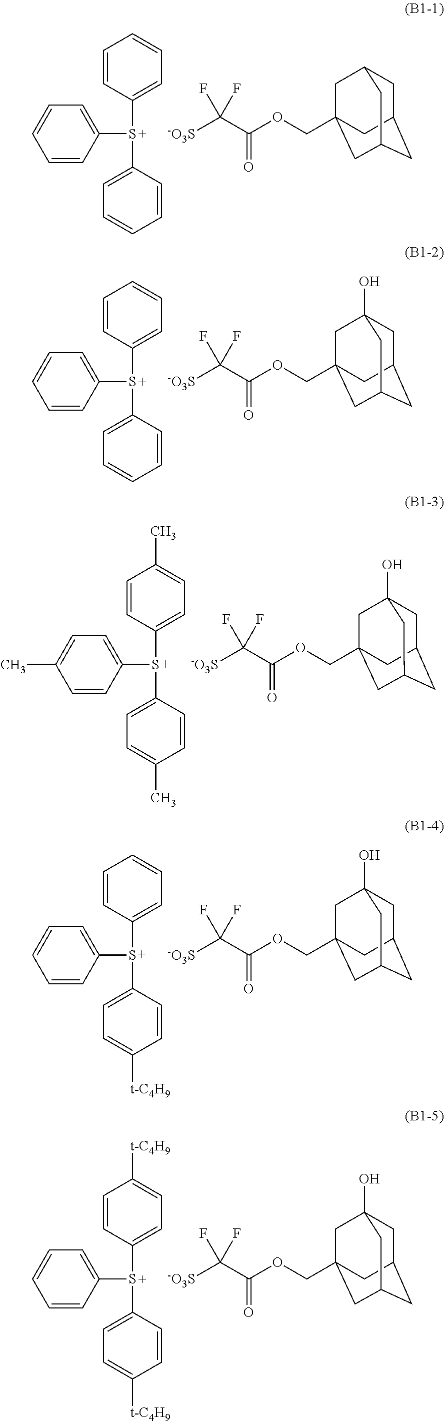

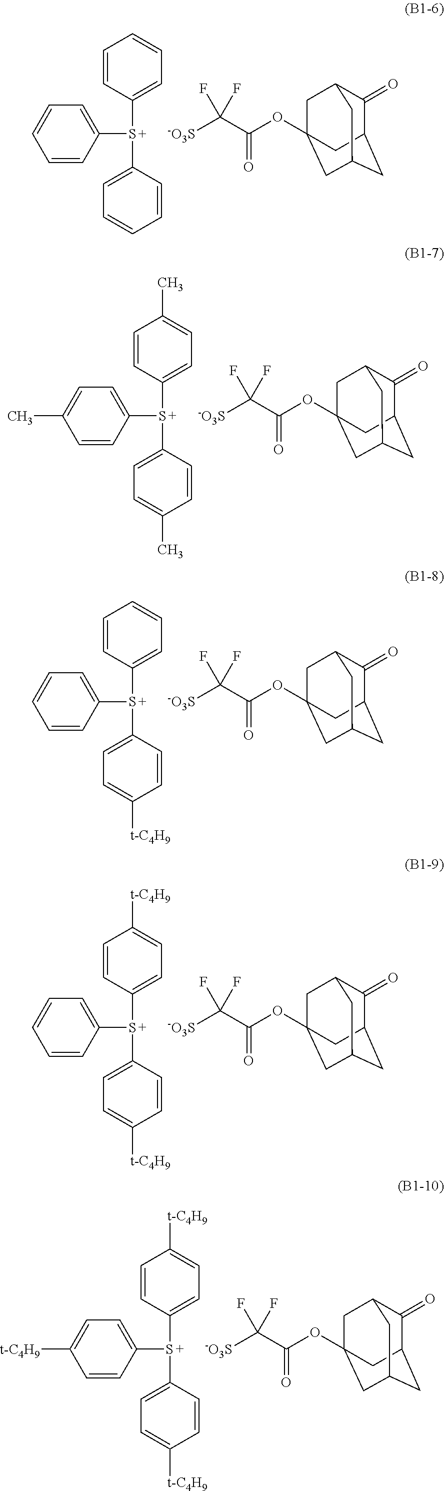

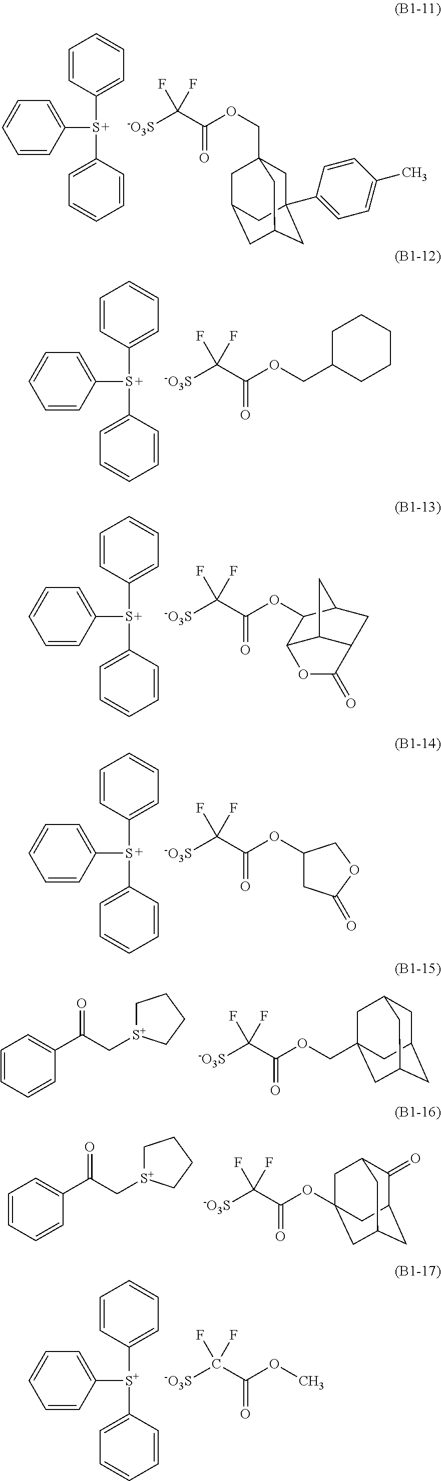

Two or more kinds of the acid generator can be used in combination.

The photoresist composition of the present invention can contain a quencher.

The quencher is preferably a basic nitrogen-containing organic compound, and examples thereof include an amine compound such as an aliphatic amine and an aromatic amine and an ammonium salt. Examples of the aliphatic amine include a primary amine, a secondary amine and a tertiary amine. Examples of the aromatic amine include an aromatic amine in which aromatic ring has one or more amino groups such as aniline and a heteroaromatic amine such as pyridine. Preferable examples thereof include an aromatic amine represented by the formula (C2):

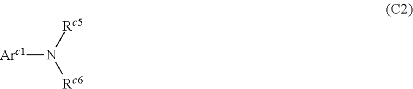

wherein Ar^{c1} represents an aromatic hydrocarbon group, and R^{c5} and R^{c6} independently represent a hydrogen atom, an aliphatic hydrocarbon group, a saturated cyclic hydrocarbon group or an aromatic hydrocarbon group, and the aliphatic hydrocarbon group, the saturated cyclic hydrocarbon group and the aromatic hydrocarbon group can have one or more substituents selected from the group consisting of a hydroxyl group, an amino group, an amino group having one or two C1-C4 alkyl groups and a C1-C6 alkoxy group.

The aliphatic hydrocarbon group is preferably an alkyl group and the saturated cyclic hydrocarbon group is preferably a cycloalkyl group. The aliphatic hydrocarbon group preferably has 1 to 6 carbon atoms. The saturated cyclic hydrocarbon group preferably has 5 to 10 carbon atoms. The aromatic hydrocarbon group preferably has 6 to 10 carbon atoms.

As the aromatic amine represented by the formula (C2), an amine represented by the formula (C2-1):

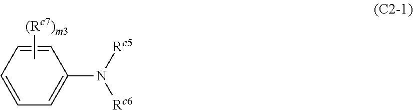

wherein R^{c5} and R^{c6} are the same as defined above, and R^{c7} is independently in each occurrence an aliphatic hydrocarbon group, an alkoxy group, a saturated cyclic hydrocarbon group or an aromatic hydrocarbon group, and the aliphatic hydrocarbon group, the alkoxy group, the saturated cyclic hydrocarbon group and the aromatic hydrocarbon group can have one or more substituents selected from the group consisting of a hydroxyl group, an amino group, an amino group having one or two C1-C4 alkyl groups and a C1-C6 alkoxy group, and m3 represents an integer of 0 to 3, is preferable. The aliphatic hydrocarbon group is preferably an alkyl group and the saturated cyclic hydrocarbon group is preferably a cycloalkyl group. The aliphatic hydrocarbon group preferably has 1 to 6 carbon atoms. The saturated cyclic hydrocarbon group preferably has 5 to 10 carbon atoms. The aromatic hydrocarbon group preferably has 6 to 10 carbon atoms. The alkoxy group preferably has 1 to 6 carbon atoms.

Examples of the aromatic amine represented by the formula (C2) include 1-naphthylamine, 2-naphthylamine, aniline, diisopropylaniline, 2-methylaniline, 3-methylaniline, 4-methylaniline, 4-nitroaniline, N-methylaniline, N,N-dimethylaniline, and diphenylamine, and among them, preferred is diisopropylaniline and more preferred is 2,6-diisopropylaniline.

Other examples of the basic compound include amines represented by the formulae (C3) to (C11):

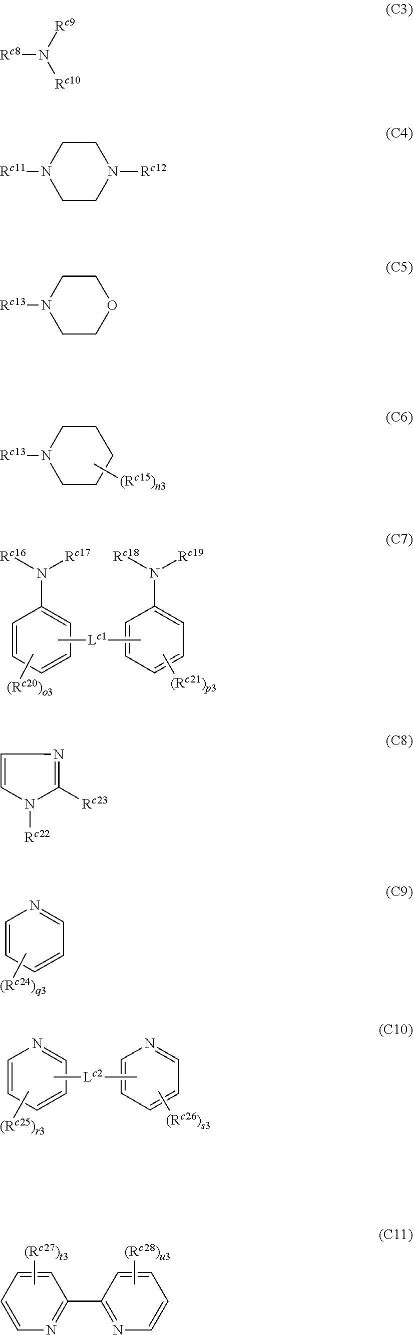

wherein R^{c8}, R^{c20}, R^{c21}, and R^{c23} to R^{c28} independently represent an aliphatic hydrocarbon group, an alkoxy group, a saturated cyclic hydrocarbon group or an aromatic hydrocarbon group, and the aliphatic hydrocarbon group, the alkoxy group, the saturated cyclic hydrocarbon group and the aromatic hydrocarbon group can have one or more substituents selected from the group consisting of a hydroxyl group, an amino group, an amino group having one or two C1-C4 alkyl groups and a C1-C6 alkoxy group,
R^{c9}, R^{c10}, R^{c11} to R^{c14}, R^{c16} to R^{c19}, and R^{c22} independently represents a hydrogen atom, an aliphatic hydrocarbon group, a saturated cyclic hydrocarbon group or an aromatic hydrocarbon group, and the aliphatic hydrocarbon group, the saturated cyclic hydrocarbon group and the aromatic hydrocarbon group can have one or more substituents selected from the group consisting of a hydroxyl group, an amino group, an amino group having one or two C1-C4 alkyl groups and a C1-C6 alkoxy group,
R^{c15} is independently in each occurrence an aliphatic hydrocarbon group, a saturated cyclic hydrocarbon group or an alkanoyl group, L^{c1} and L^{c2} independently represents a divalent aliphatic hydrocarbon group, —CO—, —C(═NH)—, —C(═NR^{c3})—, —S—, —S—S— or a combination thereof and R^{c3} represents a C1-C4 alkyl group,
O3 to u3 each independently represents an integer of 0 to 3 and n3 represents an integer of 0 to 8.

The aliphatic hydrocarbon group has preferably 1 to 6 carbon atoms, and the saturated cyclic hydrocarbon group has preferably 3 to 6 carbon atoms, and the alkanoyl group has preferably 2 to 6 carbon atoms, and the divalent aliphatic hydrocarbon group has preferably 1 to 6 carbon atoms. The divalent aliphatic hydrocarbon group is preferably an alkylene group.

Examples of the amine represented by the formula (C3) include hexylamine, heptylamine, octylamine, nonylamine, decylamine, dibutylamine, dipentylamine, dihexylamine, diheptylamine, dioctylamine, dinonylamine, didecylamine, triethylamine, trimethylamine, tripropylamine, tributylamine, tripentylamine, trihexylamine, triheptylamine, trioctylamine, trinonylamine, tridecylamine, methyldibutylamine, methyldipentylamine, methyldihexylamine, methyldicyclohexylamine, methyldiheptylamine, methyldioctylamine, methyldinonylamine, methyldidecylamine, ethyldibutylamine, ethydipentylamine, ethyldihexylamine, ethydiheptylamine, ethyldioctylamine, ethyldinonylamine, ethyldidecylamine, dicyclohexylmethylamine, tris[2-(2-methoxyethoxy)ethyl]amine, triisopropanolamine, ethylenediamine, tetramethylenediamine, hexamethylenediamine, 4,4′-diamino-1,2-diphenylethane, 4,4′-diamino-3,3′-dimethyldiphenylmethane and 4,4′-diamino-3,3′-diethyldiphenylmethane.

Examples of the amine represented by the formula (C4) include piperazine. Examples of the amine represented by the formula (C5) include morpholine. Examples of the amine represented by the formula (C6) include piperidine and hindered amine compounds having a piperidine skeleton as disclosed in JP 11-52575 A. Examples of the amine represented by the formula (C7) include 2,2′-methylenebisaniline. Examples of the amine represented by the formula (C8) include imidazole and 4-methylimidazole. Examples of the amine represented by the formula (C9) include pyridine and 4-methylpyridine. Examples of the amine represented by the formula (C10) include di-2-pyridyl ketone, 1,2-di(2-pyridyl)ethane, 1,2-di(4-pyridyl)ethane, 1,3-di(4-pyridyl)propane, 1,2-bis(2-pyridyl)ethene, 1,2-bis(4-pyridyl)ethene, 1,2-di(4-pyridyloxy)ethane, 4,4′-dipyridyl sulfide, 4,4′-dipyridyl disulfide, 2,2′-dipyridylamine and 2,2′-dipicolylamine. Examples of the amine represented by the formula (C11) include bipyridine.

Examples of the quaternary ammonium hydroxide include tetramethylammonium hydroxide, tetrabutylammonium hydroxide, tetrahexylammonium hydroxide, tetraoctylammonium hydroxide, phenyltrimethylammonium hydroxide, (3-trifluoromethylphenyl)trimethylammonium hydroxide and (2-hydroxyethyl) trimethylammonium hydroxide (so-called “choline”).

Examples of the ammonium salt include a compound represented by the formula (C12):

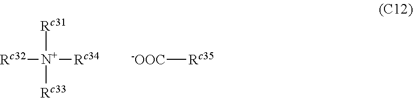

wherein R^{c31}, R^{c32}, R^{c33} and R^{c34} independently represent a C1-C20 alkyl group which may have one or more substituents, a C3-C20 saturated cyclic hydrocarbon group which may have one or more substituents or a C2-C20 alkenyl group which may have one or more substituents and R^{c35} represents a C1-C36 hydrocarbon group which may have one or more substituents and which may contain one or more heteroatoms.

Examples of the cation part of the compound represented by the formula (C12) include the cations represented by the formulae (IA-1) to (IA-8):

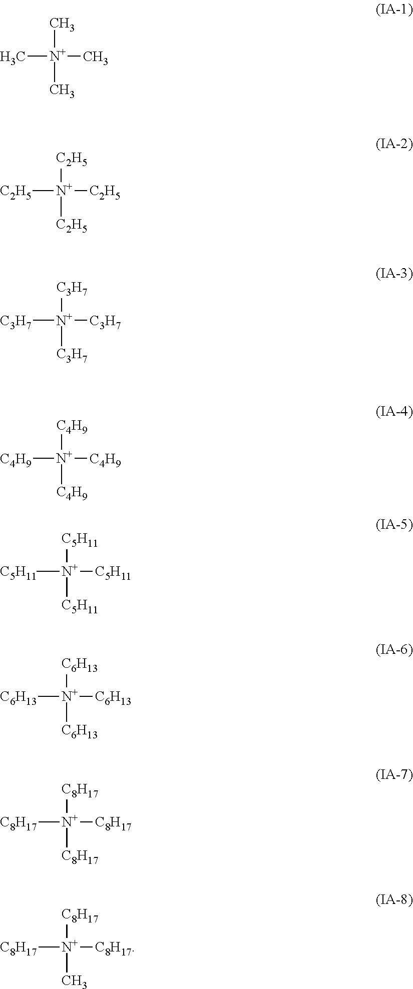

Examples of the anion part of the compound represented by the formula (C12) include the anions represented by the formulae (IB-1) to (IB-16):

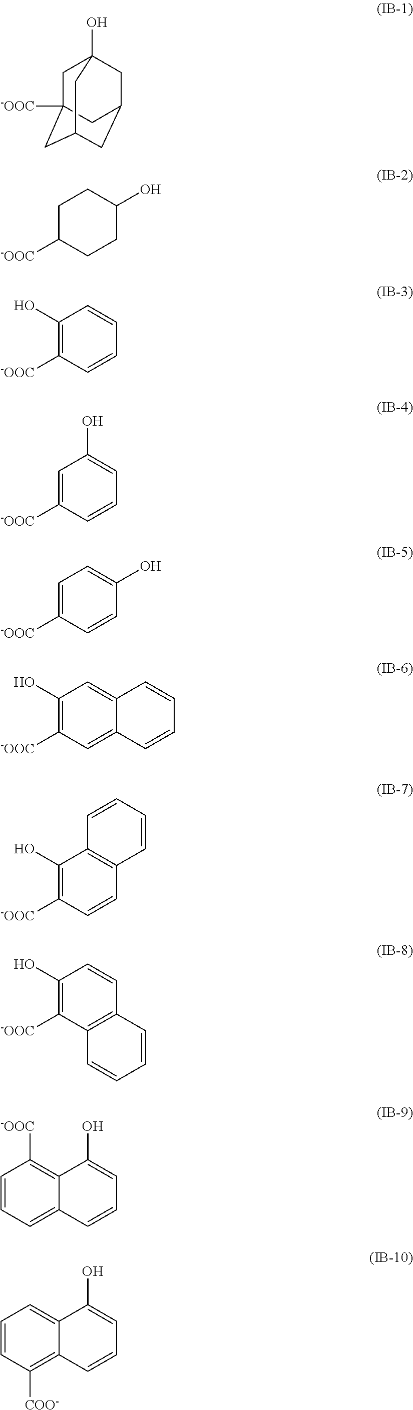

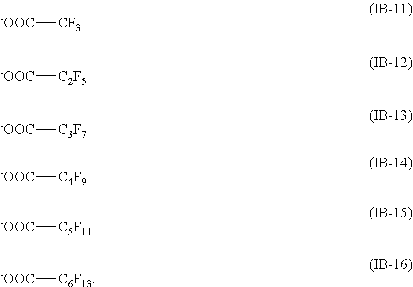

Examples of the compound represented by the formula (C12) include compounds Nos. (C12-1) to (C12-55) as shown in the following Table A, B, and C, and compounds Nos. (C12-1) to (C12-5) and (C12-12) to (C12-30) are preferable, and compound Nos. (C12-12) to (C12-21) are more preferable.

TABLE A

| Compound No. | Cation | Anion |
|---|---|---|
| (C12-1) | (IA-1) | (IB-1) |
| (C12-2) | (IA-1) | (IB-2) |
| (C12-3) | (IA-1) | (IB-3) |
| (C12-4) | (IA-1) | (IB-4) |
| (C12-5) | (IA-1) | (IB-5) |
| (C12-6) | (IA-2) | (IB-1) |
| (C12-7) | (IA-2) | (IB-2) |
| (C12-8) | (IA-2) | (IB-3) |
| (C12-9) | (IA-3) | (IB-1) |
| (C12-10) | (IA-3) | (IB-3) |
| (C12-11) | (IA-3) | (IB-5) |
| (C12-12) | (IA-4) | (IB-1) |
| (C12-13) | (IA-4) | (IB-2) |
| (C12-14) | (IA-4) | (IB-3) |
| (C12-15) | (IA-4) | (IB-4) |
| (C12-16) | (IA-4) | (IB-5) |
| (C12-17) | (IA-4) | (IB-6) |
| (C12-18) | (IA-4) | (IB-7) |
| (C12-19) | (IA-4) | (IB-8) |
| (C12-20) | (IA-4) | (IB-9) |

TABLE B

| Compound No. | Cation | Anion |
|---|---|---|
| (C12-21) | (IA-4) | (IB-10) |
| (C12-22) | (IA-5) | (IB-1) |
| (C12-23) | (IA-5) | (IB-3) |
| (C12-24) | (IA-5) | (IB-8) |
| (C12-25) | (IA-6) | (IB-1) |
| (C12-26) | (IA-6) | (IB-3) |
| (C12-27) | (IA-6) | (IB-8) |
| (C12-28) | (IA-7) | (IB-1) |
| (C12-29) | (IA-7) | (IB-3) |
| (C12-30) | (IA-7) | (IB-8) |
| (C12-31) | (IA-1) | (IB-11) |
| (C12-32) | (IA-1) | (IB-12) |
| (C12-33) | (IA-1) | (IB-13) |
| (C12-34) | (IA-1) | (IB-14) |
| (C12-35) | (IA-1) | (IB-15) |
| (C12-36) | (IA-2) | (IB-11) |
| (C12-37) | (IA-2) | (IB-12) |
| (C12-38) | (IA-2) | (IB-13) |
| (C12-39) | (IA-3) | (IB-11) |
| (C12-40) | (IA-3) | (IB-13) |
| (C12-41) | (IA-3) | (IB-15) |
| (C12-42) | (IA-4) | (IB-11) |
| (C12-43) | (IA-4) | (IB-12) |
| (C12-44) | (IA-4) | (IB-13) |
| (C12-45) | (IA-4) | (IB-14) |

TABLE C

| Compound No. | Cation | Anion |
|---|---|---|
| (C12-46) | (IA-4) | (IB-15) |
| (C12-47) | (IA-4) | (IB-16) |
| (C12-48) | (IA-5) | (IB-11) |
| (C12-49) | (IA-5) | (IB-13) |
| (C12-50) | (IA-6) | (IB-11) |
| (C12-51) | (IA-6) | (IB-13) |
| (C12-52) | (IA-7) | (IB-11) |
| (C12-53) | (IA-7) | (IB-13) |
| (C12-54) | (IA-8) | (IB-11) |
| (C12-55) | (IA-8) | (IB-13) |

When the photoresist composition contains the quencher, the content thereof is usually 0.01 to 5% by weight based on sum of solid component, and preferably 0.01 to 3% by weight and more preferably 0.01 to 1% by weight.

The photoresist composition of the present invention usually contains one or more solvents. Examples of the solvent include a glycol ether ester such as ethyl cellosolve acetate, methyl cellosolve acetate and propylene glycol monomethyl ether acetate; a glycol ether such as propylene glycol monomethyl ether; an acyclic ester such as ethyl lactate, butyl acetate, amyl acetate and ethyl pyruvate; a ketone such as acetone, methyl isobutyl ketone, 2-heptanone and cyclohexanone; and a cyclic ester such as γ-butyrolactone.

The amount of the solvent is usually 90% by weight or more, preferably 92% by weight or more preferably 94% by weight or more based on total amount of the photoresist composition of the present invention. The amount of the solvent is usually 99.9% by weight or less and preferably 99% by weight or less based on total amount of the photoresist composition of the present invention.

The photoresist composition of the present invention can contain, if necessary, a small amount of various additives such as a sensitizer, a dissolution inhibitor, other polymers, a surfactant, a stabilizer and a dye as long as the effect of the present invention is not prevented.

The photoresist composition of the present invention is useful for a chemically amplified photoresist composition.

A photoresist pattern can be produced by the following steps (1) to (5):

(1) a step of applying the first or second photoresist composition of the present invention on a substrate,

(2) a step of forming a photoresist film by conducting drying,

(3) a step of exposing the photoresist film to radiation,

(4) a step of baking the exposed photoresist film, and

(5) a step of developing the baked photoresist film with an alkaline developer, thereby forming a photoresist pattern.

The applying of the photoresist composition on a substrate is usually conducted using a conventional apparatus such as spin coater. The photoresist composition is preferably filtrated with filter having 0.2 μm of a pore size before applying. Examples of the substrate include a silicon wafer or a quartz wafer on which a sensor, a circuit, a transistor or the like is formed.

The formation of the photoresist film is usually conducted using a heating apparatus such as hot plate or a decompressor, and the heating temperature is usually 50 to 200° C., and the operation pressure is usually 1 to 1.0*10⁵ Pa.

The photoresist film obtained is exposed to radiation using an exposure system. The exposure is usually conducted through a mask having a pattern corresponding to the desired photoresist pattern. Examples of the exposure source include a light source radiating laser light in a UV-region such as a KrF excimer laser (wavelength: 248 nm), an ArF excimer laser (wavelength: 193 nm) and a F₂ laser (wavelength: 157 nm), and a light source radiating harmonic laser light in a far UV region or a vacuum UV region by wavelength conversion of laser light from a solid laser light source (such as YAG or semiconductor laser).

The temperature of baking of the exposed photoresist film is usually 50 to 200° C., and preferably 70 to 150° C.

The development of the baked photoresist film is usually carried out using a development apparatus. The alkaline developer used may be any one of various alkaline aqueous solution used in the art. Generally, an aqueous solution of tetramethylammonium hydroxide or (2-hydroxyethyl)trimethylammoniumhydroxide (commonly known as “choline”) is often used. After development, the photoresist pattern formed is preferably washed with ultrapure water, and the remained water on the photoresist pattern and the substrate is preferably removed.

The photoresist composition of the present invention is suitable for ArF excimer laser lithography, KrF excimer laser lithography, ArF immersion lithography, EUV (extreme ultraviolet) lithography and EB (electron beam) lithography, and is especially suitable for EUV lithography and EB lithography.

It should be construed that embodiments disclosed here are examples in all aspects and not restrictive. It is intended that the scope of the present invention is determined not by the above descriptions but by appended Claims, and includes all variations of the equivalent meanings and ranges to the Claims.

The present invention will be described more specifically by Examples, which are not construed to limit the scope of the present invention. The “%” used to represent the content of any component and the amount of any material to be used in the following Examples are on a weight basis unless otherwise specifically noted.

The analytical condition of liquid chromatography mass spectroscopy analysis was as followed:

LC apparatus: Agilent 1100 manufactured by Agilent Technologies, Inc.

Column: TSKgel super HZ

Mobile phase: tetrahydrofuran

Flow rate: 0.25 mL/min.

MS apparatus: HP LC-MSD 6130

Ionization: ESI⁺

Post Column: 0.5 mM NaCl/(water/methanol (1/1)) 50 μl/min. or 0.25 mM KCl/(water/acetonitrile (1/1)) 0.1 ml/min.

The analytical condition of the molecular weight analysis was as followed:

Apparatus: HLC-8120GPC manufactured by TOSOH CORPORATION

Column: TSK-GEL2000HXL and TSK-GELG4000HXL connected in series

Column temperature: 40° C.

Mobile phase: tetrahydrofuran

Flow rate: 1.0 mL/min.

Injection volume: 50 μL

Detector: RI

Sample concentration for measurement: 0.6% by weight (solvent: tetrahydrofuran)

Standard material for calibration: TSK STANDARD POLYSTYRENE F-40, F-4, F-1, A-2500, A-500 manufactured by TOSOH CORPORATION

### Reference Example 1

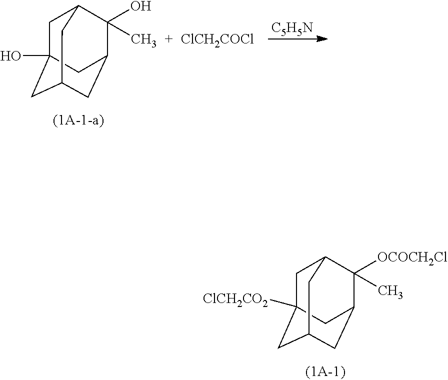

To a solution prepared by dissolving 182.3 g of a compound represented by the formula (1A-1-a) in 1,770 ml of anhydrous tetrahydrofuran, 190.8 g of pyridine was added. The resultant solution was heated up to 40° C., and then, to the solution, a solution prepared by dissolving 335.4 g of chloroacetyl chloride in 1,130 ml of anhydrous tetrahydrofuran was added dropwise over 2 hours at 51 to 63° C. The obtained mixture was stirred at 50° C. for 14 hours. The reaction mixture obtained was cooled and then, diluted with 2,396 g of 6% aqueous potassium carbonate solution and 1,268 ml of ethyl acetate to separate. The organic layer obtained was washed with pure water, and dried over anhydrous magnesium sulfate. After removing magnesium sulfate, the filtrate was concentrated under reduced pressure to obtain 287.2 g of crude product. The obtained crude product was purified with silica gel column chromatography (hexane/ethylacetate) to obtain 88.32 g of a compound represented by the formula (1A-1). Yield: 27.4%.

¹H-NMR (CDCl₃): δ=4.02 (2H, s), 3.97 (2H, s), 2.57-1.52 (13H), 1.69 (3H, s)

LC-MS: 357.1 ([M+Na]⁺; Exact Mass=334.07)

### Reference Example 2

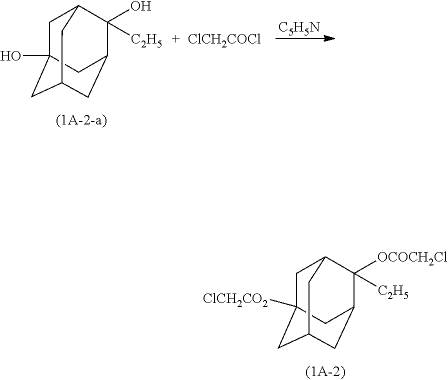

To a solution prepared by dissolving 38.12 g of a compound represented by the formula (1A-2-a) in 430 ml of anhydrous tetrahydrofuran, 76.82 g of pyridine was added. The resultant solution was heated up to 40° C., and then, to the solution, a solution prepared by dissolving 112.7 g of chloroacetyl chloride in 125 ml of anhydrous tetrahydrofuran was added dropwise over 35 minutes at 41 to 45° C. The obtained mixture was refluxed for 8 hours. The reaction mixture obtained was cooled and then, diluted with 2,396 g of 6% aqueous potassium carbonate solution and 1,268 ml of ethyl acetate to separate. The organic layer obtained was washed with pure water, and dried over anhydrous magnesium sulfate. After removing magnesium sulfate, the filtrate was concentrated under reduced pressure to obtain crude product. The obtained crude product was purified with silica gel column chromatography (hexane/ethyl acetate) to obtain 23.66 g of a compound represented by the formula (1A-2). Yield: 34.9%.

¹H-NMR (CDCl₃): δ=4.22 (2H, s), 3.98 (2H, s), 2.67-1.52 (15H), 0.84 (3H, t)

LC-MS: 371.0 ([M+Na]⁺; Exact Mass=348.09)

### Reference Example 3

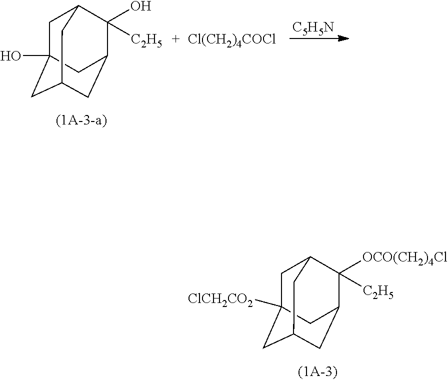

To a solution prepared by dissolving 17.04 g of a compound represented by the formula (1A-3-a) in 170 ml of anhydrous tetrahydrofuran, 34.36 g of pyridine was added. The resultant solution was heated up to 40° C., and then, to the solution, a solution prepared by dissolving 69.21 g of chloroacetyl chloride in 170 ml of anhydrous tetrahydrofuran was added dropwise over 35 minutes at 40 to 46° C. The obtained mixture was refluxed for 18 hours. The reaction mixture obtained was cooled and then, diluted with 2,396 g of 6% aqueous potassium carbonate solution and 1,268 ml of ethyl acetate to separate. The organic layer obtained was washed with pure water, and dried over anhydrous magnesium sulfate. After removing magnesium sulfate, the filtrate was concentrated under reduced pressure to obtain crude product. The obtained crude product was purified with silica gel column chromatography (hexane/ethyl acetate) to obtain 26.87 g of a compound represented by the formula (1A-3). Yield: 71.4%.

¹H-NMR (CDCl₃): δ=3.59-3.53 (4H), 2.57-1.50 (27H), 0.82 (3H, t)

LC-MS: 455.1 ([M+Na]⁺; Exact Mass=432.18)

The compounds used in the following Examples are as followed.

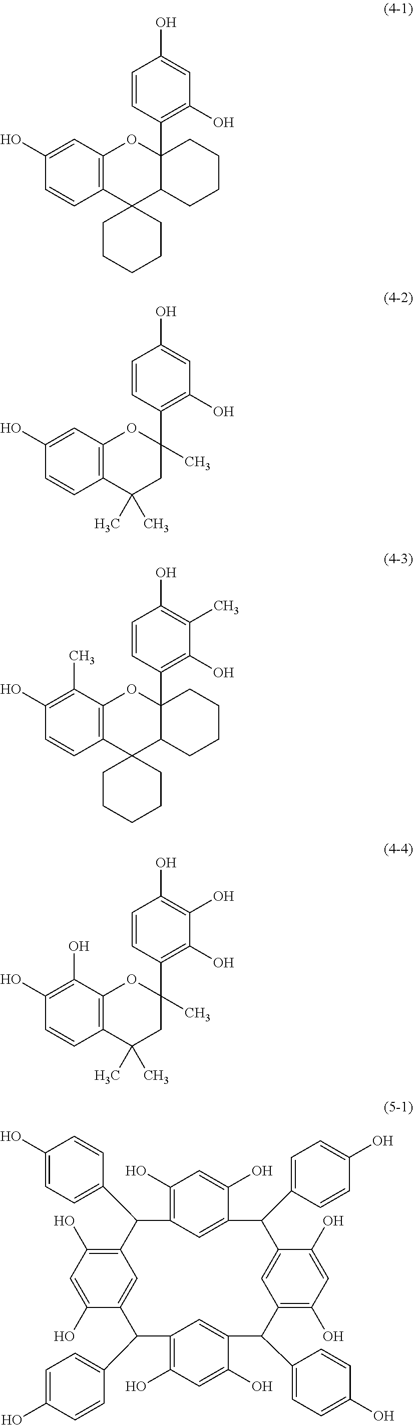

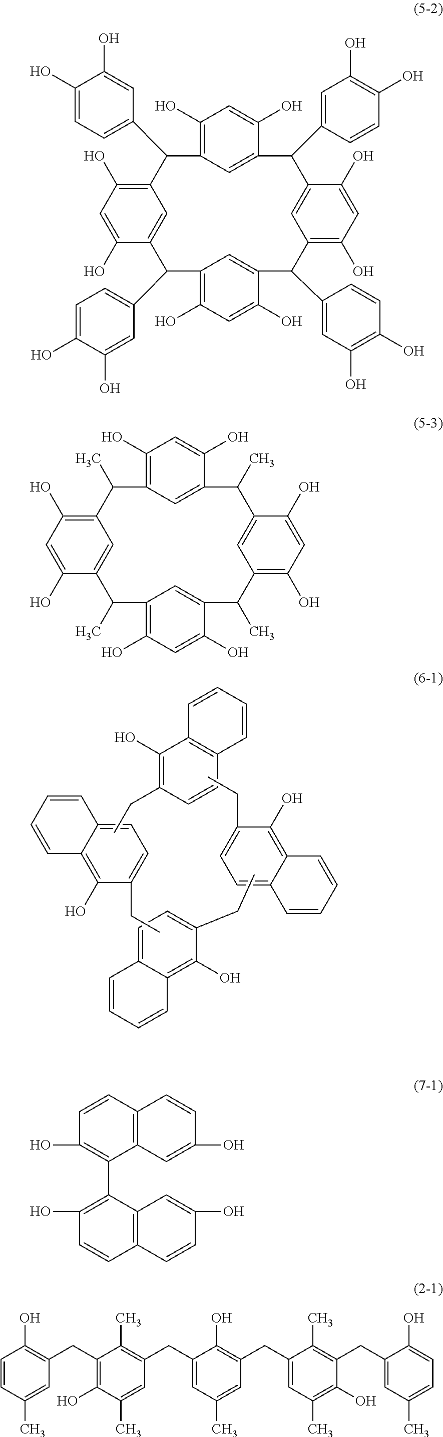

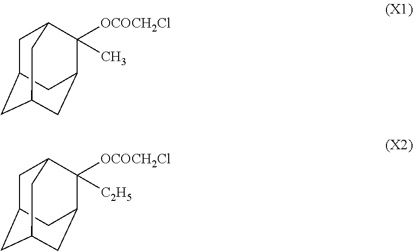

### Example 1

To a solution prepared by dissolving 3.90 g of the compound represented by the formula (4-1) in 78 ml of anhydrous N,N-dimethylformamide, 4.29 g of potassium carbonate and 0.69 g of potassium iodide were added. To the resultant mixture, a solution prepared by dissolving 3.47 g of the compound represented by the formula (1A-1) in 20 ml of anhydrous N,N-dimethylformamide was added dropwise over 30 minutes at 70° C. The obtained mixture was stirred at 70° C. for 6 hours. The reaction mixture obtained was cooled, and then, acidified (pH=1 to 2) with 2% aqueous oxalic acid solution followed by conducting extraction with ethyl acetate. The organic layer obtained was washed with pure water until becoming neutral. The organic layer was mixed with activated carbon to be decolorized. The obtained mixture was filtrated and the filtrate was concentrated to obtain 5.20 g of a resin, which is called as resin A1. Mw: 1.2×10⁴, Mw/Mn: 7.560

### Example 2

To a solution prepared by dissolving 3.10 g of the compound represented by the formula (4-2) in 62 ml of anhydrous N,N-dimethylformamide, 4.32 g of potassium carbonate was added. To the resultant mixture, a solution prepared by dissolving 3.49 g of the compound represented by the formula (1A-1) in 16 ml of anhydrous N,N-dimethylformamide was added dropwise over 30 minutes at room temperature. To the obtained mixture, 0.69 g of potassium iodide was added and then, the resultant mixture was stirred at 70° C. for 10.5 hours. The reaction mixture obtained was cooled, and then, acidified (pH=1 to 2) with 2% aqueous oxalic acid solution followed by conducting extraction with ethyl acetate. The organic layer obtained was washed with pure water until becoming neutral. The organic layer was mixed with activated carbon to be decolorized. The obtained mixture was filtrated and the filtrate was concentrated to obtain 5.75 g of a resin, which is called as resin A2. Mw: 1.2×10⁴, Mw/Mn: 6.167

### Example 3

To a solution prepared by dissolving 3.70 g of the compound represented by the formula (4-3) in 74 ml of anhydrous N,N-dimethylformamide, 3.79 g of potassium carbonate and 0.61 g of potassium iodide were added. To the resultant mixture, a solution prepared by dissolving 3.07 g of the compound represented by the formula (1A-1) in 19 ml of anhydrous N,N-dimethylformamide was added dropwise over 45 minutes at 70° C. The obtained mixture was stirred at 70° C. for 4 hours. The reaction mixture obtained was cooled, and then, acidified (pH=1 to 2) with 2% aqueous oxalic acid solution followed by conducting extraction with ethyl acetate. The organic layer obtained was washed with pure water until becoming neutral. The organic layer was mixed with activated carbon to be decolorized. The obtained mixture was filtrated and the filtrate was concentrated to obtain 5.92 g of a resin, which is called as resin A3. Mw: 6.0×10³, Mw/Mn: 3.950

### Example 4

To a solution prepared by dissolving 2.00 g of the compound represented by the formula (4-4) in 40 ml of anhydrous N,N-dimethylformamide, 5.04 g of potassium carbonate and 0.40 g of potassium iodide were added. To the resultant mixture, a solution prepared by dissolving 4.08 g of the compound represented by the formula (1A-1) in 10 ml of anhydrous N,N-dimethylformamide was added dropwise over 40 minutes at 70° C. The obtained mixture was stirred at 70° C. for 5.5 hours. The reaction mixture obtained was cooled, and then, acidified (pH=1 to 2) with 2% aqueous oxalic acid solution followed by conducting extraction with ethyl acetate. The organic layer obtained was washed with pure water until becoming neutral. The organic layer was mixed with activated carbon to be decolorized. The obtained mixture was filtrated and the filtrate was concentrated to obtain 1.48 g of a resin, which is called as resin A4. Mw: 6.0×10³, Mw/Mn: 4.714

### Example 5

To a solution prepared by dissolving 4.00 g of the compound represented by the formula (5-1) in 80 ml of anhydrous N,N-dimethylformamide, 3.91 g of potassium carbonate and 0.31 g of potassium iodide were added. To the resultant mixture, a solution prepared by dissolving 3.16 g of the compound represented by the formula (1A-1) in 20 ml of anhydrous N,N-dimethylformamide was added dropwise over 40 minutes at 70° C. The obtained mixture was stirred at 70° C. for 4 hours. The reaction mixture obtained was cooled, and then, diluted with 189 ml of methanol and then, the resultant mixture was acidified (pH=3) with 378 ml of 1% aqueous oxalic acid solution followed by stirring overnight at room temperature. The mixture was filtrated and then, the solid obtained was washed with pure water until becoming neutral. The solid was dried at 80° C. under reduced pressure to obtain 6.10 g of a resin, which is called as resin A5. Mw: 1.2×10⁴, Mw/Mn: 4.404

### Example 6

To a solution prepared by dissolving 3.00 g of the compound represented by the formula (5-2) in 60 ml of anhydrous N,N-dimethylformamide, 5.46 g of potassium carbonate and 0.22 g of potassium iodide were added. To the resultant mixture, a solution prepared by dissolving 4.41 g of the compound represented by the formula (1A-1) in 15 ml of anhydrous N,N-dimethylformamide was added dropwise over 40 minutes at 70° C. The obtained mixture was stirred at 70° C. for 4 hours. The reaction mixture obtained was cooled, and then, diluted with 264 ml of methanol and then, the resultant mixture was acidified (pH=2) with 528 ml of 1% aqueous oxalic acid solution followed by stirring overnight at room temperature. The mixture was filtrated and then, the solid obtained was washed with pure water until becoming neutral. The solid was dried at 80° C. under reduced pressure to obtain 4.82 g of a resin, which is called as resin A6. Mw: 1.5×10³, Mw/Mn: 2.040

### Example 7

To a solution prepared by dissolving 4.00 g of the compound represented by the formula (6-1) in 80 ml of anhydrous N,N-dimethylformamide, 2.68 g of potassium carbonate and 0.43 g of potassium iodide were added. To the resultant mixture, a solution prepared by dissolving 2.17 g of the compound represented by the formula (1A-1) in 20 ml of anhydrous N,N-dimethylformamide was added dropwise over 40 minutes at 70° C. The obtained mixture was stirred at 70° C. for 4 hours. The reaction mixture obtained was cooled, and then, diluted with 130 ml of methanol and then, the resultant mixture was acidified (pH=2) with 259 ml of 1% aqueous oxalic acid solution followed by stirring for 1 hour at room temperature. The mixture was filtrated and then, the solid obtained was washed with pure water until becoming neutral. The solid was dried at 80° C. under reduced pressure to obtain 5.12 g of a resin, which is called as resin A7. Mw: 1.7×10⁴, Mw/Mn: 8.233

### Example 8

To a solution prepared by dissolving 3.00 g of the compound represented by the formula (7-1) in 60 ml of anhydrous N,N-dimethylformamide, 3.95 g of potassium carbonate and 0.63 g of potassium iodide were added. To the resultant mixture, a solution prepared by dissolving 3.19 g of the compound represented by the formula (1A-1) in 15 ml of anhydrous N,N-dimethylformamide was added dropwise over 40 minutes at 70° C. The obtained mixture was stirred at 70° C. for 5.3 hours. The reaction mixture obtained was cooled, and then, diluted with 191 ml of methanol and then, the resultant mixture was acidified (pH=2) with 191 ml of 2% aqueous oxalic acid solution followed by stirring for 1 hour at room temperature. The mixture was filtrated and then, the solid obtained was washed with pure water until becoming neutral. The solid was dried at 80° C. under reduced pressure to obtain 4.90 g of a resin, which is called as resin A8. Mw: 4.9×10³, Mw/Mn: 2.291

### Example 9

To a solution prepared by dissolving 2.7 g of the compound represented by the formula (5-3) in 70 ml of anhydrous N,N-dimethylformamide, 5.0 g of the compound represented by the formula (1A-1) was added. To the resultant mixture, 6.2 g of potassium carbonate and 0.20 g of potassium iodide were added. The obtained mixture was stirred at 75° C. for 15 hours. The reaction mixture obtained was cooled, and then, acidified (pH=2) with 400 ml of 2% aqueous oxalic acid solution followed by stirring for 1 hour at room temperature. The mixture was filtrated and then, the solid obtained was washed with pure water until becoming neutral. The solid was dried at 40° C. under reduced pressure to obtain 6.30 g of a resin, which is called as resin A9. Mw: 3.3×10³, Mw/Mn: 4.286

### Example 10

To a solution prepared by dissolving 3.00 g of the compound represented by the formula (7-1) in 60 ml of anhydrous N,N-dimethylformamide, 3.95 g of potassium carbonate and 0.63 g of potassium iodide were added. To the resultant mixture, a solution prepared by dissolving 3.32 g of the compound represented by the formula (1A-2) in 15 ml of anhydrous N,N-dimethylformamide was added dropwise over 45 minutes at 70° C. The obtained mixture was stirred at 70° C. for 8 hours. The reaction mixture obtained was cooled, and then, diluted with 191 ml of methanol and then, the resultant mixture was acidified (pH=2) with 382 ml of 1% aqueous oxalic acid solution followed by stirring for 1 hour at room temperature. The mixture was filtrated and then, the solid obtained was washed with pure water until becoming neutral. The solid was dried at 80° C. under reduced pressure to obtain 4.25 g of a resin, which is called as resin A10. Mw: 2.3×10³, Mw/Mn: 1.924

### Example 11

To a solution prepared by dissolving 5.0 g of the compound represented by the formula (5-3) in 50 ml of anhydrous N,N-dimethylformamide, 6.4 g of the compound represented by the formula (1A-2) was added. To the resultant mixture, 3.8 g of potassium carbonate and 0.30 g of potassium iodide were added. The obtained mixture was stirred at 75° C. for 9 hours. The reaction mixture obtained was cooled, and then, acidified (pH=3) with 200 ml of 2% aqueous oxalic acid solution followed by conducting extraction with ethyl acetate. The organic layer obtained was washed with pure water until becoming neutral. The organic layer was dried over anhydrous magnesium sulfate. The obtained mixture was filtrated and the filtrate was concentrated under reduced pressure to obtain 8.0 g of a resin, which is called as resin A11. Mw: 7.0×10³, Mw/Mn: 3.140

### Example 12

(1) To a solution prepared by dissolving 50.05 g of the compound represented by the formula (5-3) in 500 ml of anhydrous N,N-dimethylformamide, 89.11 g of the compound represented by the formula (X1) was added. To the resultant mixture, 76.00 g of potassium carbonate and 3.14 g of potassium iodide were added. The obtained mixture was stirred at 75° C. for 8 hours. The reaction mixture obtained was cooled, and then, acidified (pH=5) with 1,330 g of 5% aqueous oxalic acid solution followed by conducting extraction with ethyl acetate. The organic layer obtained was washed with pure water until becoming neutral. The organic layer was dried over anhydrous magnesium sulfate. The obtained mixture was filtrated and the filtrate was concentrated under reduced pressure to obtain 125.19 g of a mixture of the following compounds represented by the formulae (5-3-X1-1), (5-3-X1-2) and (5-3-X1-3):

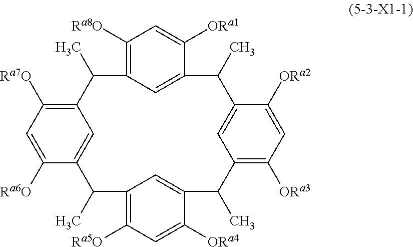

wherein any three of R^{a1}, R^{a2}, R^{a3}, R^{a4}, R^{a5}, R^{a6}, R^{a7} and R^{a8} are the group represented by the following formula (X1-1):

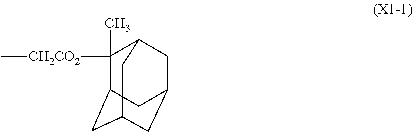

and the other five groups are hydrogen atoms,

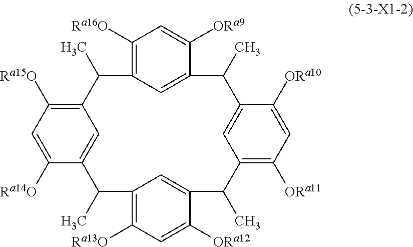

wherein any four of R^{a9}, R^{a10}, R^{a11}, R^{a12}, R^{a13}, R^{a14}, R^{a15} and R^{a16} are the group represented by the formula (X1-1) and the other four groups are hydrogen atoms,

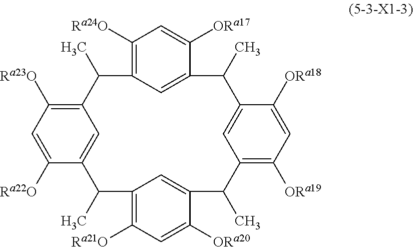

wherein any five of R^{a17}, R^{a18}, R^{a19}, R^{a20}, R^{a21}, R^{a22}, R^{a23} and R^{a24} are the group represented by the formula (X1-1) and the other three groups are hydrogen atoms, which is called as P1.

The content ratio of the compounds represented by the formulae (5-3-X1-1), (5-3-X1-2) and (5-3-X1-3) in P1 (compound represented by the formula (5-3-X1-1): compound represented by the formula (5-3-X1-2): compound represented by the formula (5-3-X1-3)) was 17:76:7.

The compound represented by the formula (5-3-X1-1): [M+Na]=1187.6 (C₇₁H₈₆O₁₄=1163.43)

The compound represented by the formula (5-3-X1-2): [M+Na]=1392.7 (C₈₄H₁₀₄O₁₆=1369.71)

The compound represented by the formula (5-3-X1-3): [M+Na]=1599.8 (C₉₇H₁₂₂O₁₈=1576.00)

(2) To a solution prepared by dissolving 4.50 g of P1 in 90 ml of anhydrous N,N-dimethylformamide, 1.44 g of the compound represented by the formula (1A-3) was added. To the resultant mixture, 1.38 g of potassium carbonate and 0.22 g of potassium iodide were added. The obtained mixture was stirred at 75° C. for 21 hours. The reaction mixture obtained was cooled, and then, acidified (pH=3) with 133 ml of 2% aqueous oxalic acid solution followed by conducting extraction with ethyl acetate. The organic layer obtained was washed with pure water until becoming neutral. The organic layer was dried over anhydrous magnesium sulfate. The obtained mixture was filtrated and the filtrate was concentrated under reduced pressure to obtain 6.75 g of a resin, which is called as resin A12. Mw: 2.2×10³, Mw/Mn: 2.463

### Example 13

(1) To a solution prepared by dissolving 10 g of the compound represented by the formula (2-1) in 100 g of anhydrous N,N-dimethylformamide, 5.2 g of potassium carbonate was added. To the resultant mixture, a solution prepared by dissolving 5.9 g of the compound represented by the formula (X1) in 40 g of anhydrous N,N-dimethylformamide was added dropwise below 50° C. To the obtained mixture, 0.4 g of potassium iodide was added. The obtained mixture was stirred at 50° C. for 5 hours. The reaction mixture obtained was cooled, and then, diluted with 1% aqueous oxalic acid solution followed by conducting extraction with ethyl acetate. The organic layer obtained was washed with water followed by conducting drying over anhydrous magnesium sulfate and decolorizing with active carbon. The obtained mixture was filtrated and the filtrate was concentrated to obtain 11.94 g of a mixture of the compound represented by the formula (2-1) and the following compounds represented by the formulae (2-1-X1-1) and (2-1-X1-2):

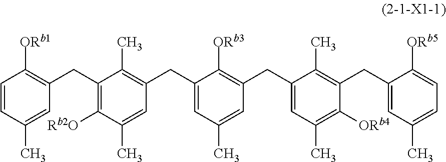

wherein any one of R^{b1}, R^{b2}, R^{b3}, R^{b4} and R^{b5} is the group represented by the formula (X1-1) and the other four groups are hydrogen atoms,

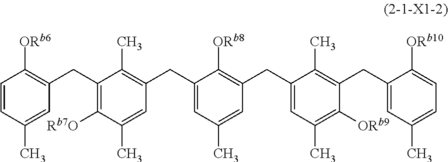

wherein any two of R^{b6}, R^{b7}, R^{b8}, R^{b9} and R^{b10} are the group represented by the formula (X1-1) and the other three groups are hydrogen atoms, which is called as P2.

The content ratio of the compounds represented by the formulae (2-1), (2-1-X1-1) and (2-1-X1-2) in P2 (compound represented by the formula (2-1): compound represented by the formula (2-1-X1-1): compound represented by the formula (2-1-X1-2)) was 4:29:67.

The compound represented by the formulae (2-1): [M+K]⁺=655.3 (M⁺=616.32)

The compound represented by the formulae (2-1-X1-1): [M+K]⁺=861.4 (M⁺=822.45)

The compound represented by the formulae (2-1-X1-2): [M+K]⁺=1067.4 (M⁺=1028.58)

(2) To a solution prepared by dissolving 5.0 g of P2 in 25 ml of anhydrous N, N-dimethylformamide, 1.0 g of the compound represented by the formula (1A-1) was added. To the resultant mixture, 0.9 g of potassium carbonate and 0.10 g of potassium iodide were added. The obtained mixture was stirred at 53° C. for 5.5 hours. The reaction mixture obtained was cooled, and then, acidified (pH=3) with 60 ml of 2% aqueous oxalic acid solution followed by conducting extraction with ethyl acetate. The organic layer obtained was washed with pure water (pH=5). The organic layer was dried over anhydrous magnesium sulfate. The obtained mixture was filtrated and the filtrate was concentrated under reduced pressure to obtain 5.3 g of a resin, which is called as resin A13. Mw: 4.0×10³, Mw/Mn: 2.005

### Example 14

(1) To a solution prepared by dissolving 50.80 g of the compound represented by the formula (2-1) in 435 g of anhydrous N,N-dimethylformamide, 32.88 g of potassium carbonate was added. To the resultant mixture, a solution prepared by dissolving 43.50 g of the compound represented by the formula (X2) in 218 g of anhydrous N,N-dimethylformamide was added dropwise over 15 minutes at 23 to 25° C. To the obtained mixture, 2.63 g of potassium iodide was added. The obtained mixture was stirred at 50 to 51° C. for 5 hours. The reaction mixture obtained was cooled, and then, diluted with 5% aqueous oxalic acid solution followed by conducting extraction with ethyl acetate. The organic layer obtained was washed with water followed by conducting drying over anhydrous magnesium sulfate and decolorizing with active carbon. The obtained mixture was filtrated and the filtrate was concentrated to obtain 65.76 g of a mixture of the following compounds represented by the formulae (2-1-X2-1), (2-1-X2-2) and (2-1-X2-3):

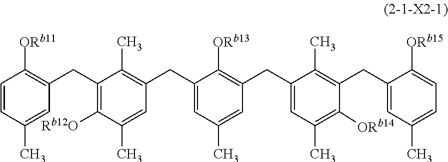

wherein any one of R^{b11}, R^{b12}, R^{b13}, R^{b14} and R^{b15} is the group represented by the formula (X2-1)

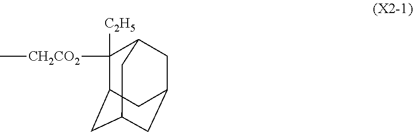

and the other four groups are hydrogen atoms,

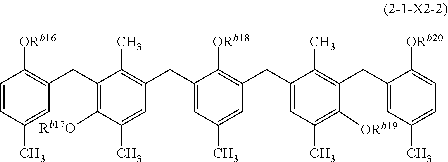

wherein any two of R^{b16}, R^{b17}, R^{b18}, R^{b19} and R^{b20} are the group represented by the formula (X2-1) and the other three groups are hydrogen atoms,

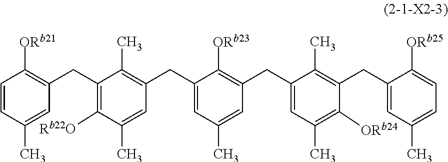

wherein any three of R^{b21}, R^{b22}, R^{b23}, R^{b24} and R^{b25} are the group represented by the formula (X2-1) and the other two groups are hydrogen atoms, which is called as P3.

The content ratio of the compounds represented by the formulae (2-1-X2-1), (2-1-X2-2) and (2-1-X2-3) in P3 (compound represented by the formula (2-1-X2-1): compound represented by the formula (2-1-X2-2): compound represented by the formula (2-1-X2-3)) was 12:84:4.

The compound represented by the formulae (2-1-X2-1): [M+K]⁺=755.2 (M⁺=716.41)

The compound represented by the formulae (2-1-X2-2): [M+K]⁺=1095.4 (M⁺=1056.61)

The compound represented by the formulae (2-1-X2-3): [M+K]⁺=1315.5 (M⁺=1276.76)

(2) To a solution prepared by dissolving 5.0 g of P3 in 50 ml of anhydrous N, N-dimethylformamide, 2.4 g of the compound represented by the formula (1A-1) was added. To the resultant mixture, 3.0 g of potassium carbonate and 0.20 g of potassium iodide were added. The obtained mixture was stirred at 75° C. for 11 hours. The reaction mixture obtained was cooled, and then, acidified (pH=3) with 60 ml of 2% aqueous oxalic acid solution followed by conducting extraction with chloroform. The organic layer obtained was washed with pure water until becoming neutral. The organic layer was dried over anhydrous magnesium sulfate. The obtained mixture was filtrated and the filtrate was concentrated under reduced pressure to obtain 5.5 g of a resin, which is called as resin A14. Mw: 1.5×10⁴, Mw/Mn: 4.831

### Example 15

(1) To a solution prepared by dissolving 50.05 g of the compound represented by the formula (5-3) in 500 ml of anhydrous N,N-dimethylformamide, 89.11 g of the compound represented by the formula (X1) was added. To the resultant mixture, 76.00 g of potassium carbonate and 3.14 g of potassium iodide were added. The obtained mixture was stirred at 75° C. for 8 hours. The reaction mixture obtained was cooled, and then, acidified (pH=5) with 1,330 g of 5% aqueous oxalic acid solution followed by conducting extraction with ethyl acetate. The organic layer obtained was washed with pure water until becoming neutral. The organic layer was dried over anhydrous magnesium sulfate. The obtained mixture was filtrated and the filtrate was concentrated under reduced pressure to obtain 125.19 g of a mixture of the following compounds represented by the formulae (5-3-X1-1), (5-3-X1-2) and (5-3-X1-3):

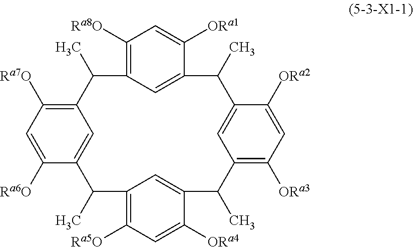

wherein any three of R^{a1}, R^{a2}, R^{a3}, R^{a4}, R^{a5}, R^{a6}, R^{a7} and R^{a8} are the group represented by the formula (X1-1) and the other five groups are hydrogen atoms,

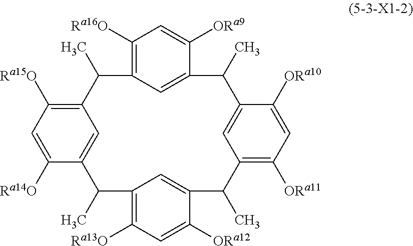

wherein any four of R^{a9}, R^{a10}, R^{a11}, R^{a12}, R^{a13}, R^{a14}, R^{a15} and R^{a16} are the group represented by the formula (X1-1) and the other four groups are hydrogen atoms,

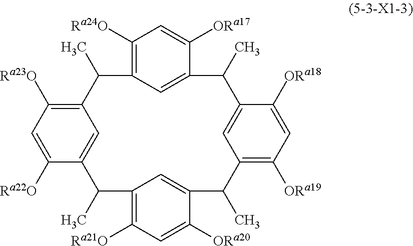

wherein any five of R^{a17}, R^{a18}, R^{a19}, R^{a20}, R^{a21}, R^{a22}, R^{a23} and R^{a24} are the group represented by the formula (X1-1) and the other three groups are hydrogen atoms, which is called as P1.

The content ratio of the compounds represented by the formulae (5-3-X1-1), (5-3-X1-2) and (5-3-X1-3) in P1 (compound represented by the formula (5-3-X1-1): compound represented by the formula (5-3-X1-2): compound represented by the formula (5-3-X1-3)) was 17:76:7.

The compound represented by the formula (5-3-X1-1): [M+Na]=1187.6 (C₇₁H₆₆O₁₄=1163.43)

The compound represented by the formula (5-3-X1-2): [M+Na]=1392.7 (C₈₄H₁₀₄O₁₆=1369.71)

The compound represented by the formula (5-3-X1-3): [M+Na]=1599.8 (C₉₇H₁₂₂O₁₈=1576.00)

(2) To a solution prepared by dissolving 5.00 g of P1 in 25 ml of anhydrous N,N-dimethylformamide, 0.61 g of the compound represented by the formula (1A-1) was added. To the resultant mixture, 0.61 g of potassium carbonate and 0.10 g of potassium iodide were added. The obtained mixture was stirred at 53 to 54° C. for 7 hours. The reaction mixture obtained was cooled, and then, acidified (pH=3) with 50 ml of 1% aqueous oxalic acid solution followed by conducting extraction with ethyl acetate. The organic layer obtained was washed with pure water until becoming neutral. The organic layer was dried over anhydrous magnesium sulfate. The obtained mixture was filtrated and the filtrate was concentrated under reduced pressure to obtain 4.30 g of a resin, which is called as resin A15. Mw: 2.7×10³, Mw/Mn: 1.587

### Example 16

(1) To a solution prepared by dissolving 10.0 g of the compound represented by the formula (5-3) in 50 ml of anhydrous N,N-dimethylformamide, 18.9 g of the compound represented by the formula (X2) was added. To the resultant mixture, 12.2 g of potassium carbonate and 0.60 g of potassium iodide were added. The obtained mixture was stirred at 75° C. for 4.5 hours. The reaction mixture obtained was cooled, and then, acidified (pH=3) with 400 g of 2% aqueous oxalic acid solution followed by conducting extraction with ethyl acetate. The organic layer obtained was washed with pure water until becoming neutral. The organic layer was dried over anhydrous magnesium sulfate. The obtained mixture was filtrated and the filtrate was concentrated under reduced pressure to obtain 26.1 g of a mixture of the following compounds represented by the formulae (5-3-X2-1), (5-3-X2-2), (5-3-X2-3) and (5-3-X2-4):

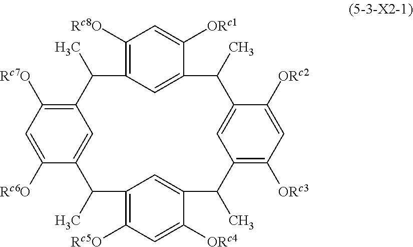

wherein any two of R^{c1}, R^{c2}, R^{c3}, R^{c4}, R^{c5}, R^{c6}, R^{c7} and R^{c8} are the group represented by the formula (X2-1) and the other six groups are hydrogen atoms,

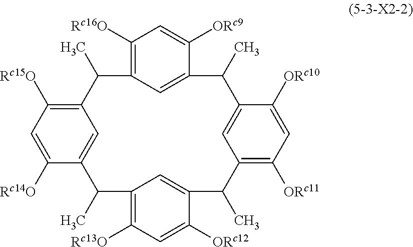

wherein any three of R^{c9}, R^{c10}, R^{c11}, R^{c12}, R^{c13}, R^{c14}, R^{c15} and R^{c16} are the group represented by the formula (X2-1) and the other five groups are hydrogen atoms,

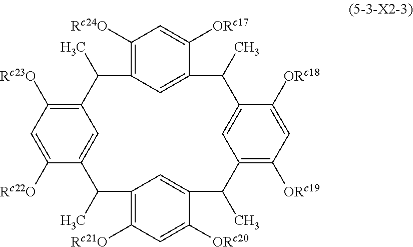

wherein any four of R^{c17}, R^{c18}, R^{c19}, R^{c20}, R^{c21}, R^{c22}, R^{c23} and R^{c24} are the group represented by the formula (X2-1) and the other four groups are hydrogen atoms,

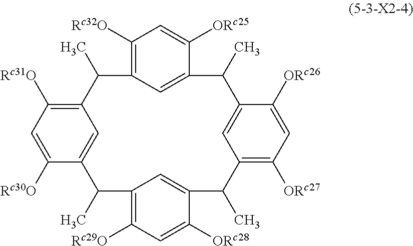

wherein any five of R^{c25}, R^{c26}, R^{c27}, R^{c28}, R^{c29}, R^{c30}, R^{c31} and R^{c32} are the group represented by the formula (X2-1) and the other three groups are hydrogen atoms, which is called as P4.

The content ratio of the compounds represented by the formulae (5-3-X2-1), (5-3-X2-2), (5-3-X2-3) and (5-3-X2-4) in P4 (compound represented by the formula (5-3-X2-1): compound represented by the formula (5-3-X2-2): compound represented by the formula (5-3-X2-3): compound represented by the formula (5-3-X2-4)) was 5:21:65:9.

The compound represented by the formula (5-3-X2-1): [M+Na]=1007.3 (C₆₀H₇₂O₁₂=984.5)

The compound represented by the formula (5-3-X2-2): [M+Na]=1227.5 (C₇₄H₉₂O₁₄=1204.65)

The compound represented by the formula (5-3-X2-3): [M+Na]=1447.6 (C₈₈H₁₁₂O₁₆=1424.8)

The compound represented by the formula (5-3-X2-4): [M+Na]=1668.7 (C₁₀₂H₁₃₂O₁₈=1644.94)

(2) To a solution prepared by dissolving 7.00 g of P4 in 35 ml of anhydrous N,N-dimethylformamide, 0.83 g of the compound represented by the formula (1A-1) was added. To the resultant mixture, 1.00 g of potassium carbonate and 0.20 g of potassium iodide were added. The obtained mixture was stirred at 75° C. for 5 hours. The reaction mixture obtained was cooled, and then, acidified (pH=3) with 100 ml of 1% aqueous oxalic acid solution followed by conducting extraction with ethyl acetate. The organic layer obtained was washed with pure water until becoming neutral. The organic layer was dried over anhydrous magnesium sulfate. The obtained mixture was filtrated and the filtrate was concentrated under reduced pressure to obtain 5.80 g of a resin, which is called as resin A16. Mw: 3.9×10³, Mw/Mn: 1.870

### Example 17

(1) To a solution prepared by dissolving 60.0 g of the compound represented by the formula (4-2) in 300 g of anhydrous N,N-dimethylformamide, 72.7 g of the compound represented by the formula (X1) was added. To the resultant mixture, 63.5 g of potassium carbonate and 3.3 g of potassium iodide were added. The obtained mixture was stirred at 80 to 84° C. for 6.5 hours. The reaction mixture obtained was cooled, and then, acidified (pH=3) with 87 g of 5% aqueous oxalic acid solution followed by conducting extraction with ethyl acetate. The organic layer obtained was washed with pure water until becoming neutral. The organic layer was dried over anhydrous magnesium sulfate. The obtained mixture was filtrated and the filtrate was concentrated under reduced pressure to obtain 141 g of a mixture of the compound represented by the formula (4-2) and the following compounds represented by the formulae (4-2-X1-1), (4-2-X1-2) and (4-2-X1-3):

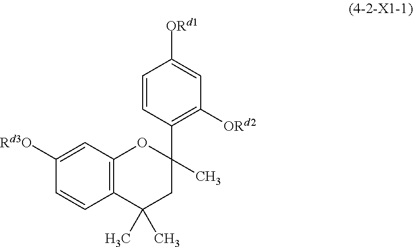

wherein any one of R^{d1}, R^{d2} and R^{d3} is the group represented by the formula (X1-1) and the other two groups are hydrogen atoms,

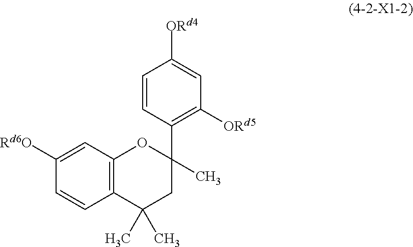

wherein any two of R^{d4}, R^{d5} and R^{d6} are the groups represented by the formula (X1-1) and the other one group is a hydrogen atom,

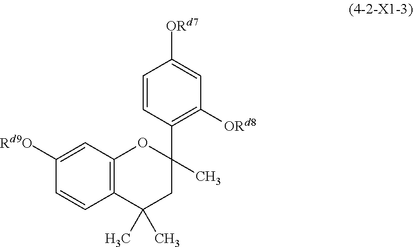

wherein R^{d7}, R^{d8} and R^{d9} are the groups represented by the formula (X1-1), which is called as P5.

The content ratio of the compounds represented by the formulae (4-2), (4-2-X1-1), (4-2-X1-2) and (4-2-X1-3) in P5 (compound represented by the formula (4-2): compound represented by the formula (4-2-X1-1): compound represented by the formula (4-2-X1-2): compound represented by the formula (4-2-X1-3)) was 17:31:26:26.

P5 was purified with silica gel column chromatography (hexane/ethyl acetate) to obtain 13.0 g of the compound represented by the formula (4-2) (Recovery rate: 21.7%), 23.9 g of the compound represented by the formula (4-2-X1-1) (Yield: 23.6%), 10.2 g of the compound represented by the formula (4-2-X1-2) (Yield: 7.2%) and 14.0 g of the compound represented by the formula (4-2-X1-3) (Yield: 7.6%).

The compound represented by the formula (4-2-X1-1): [M+Na]=529.2 (C₃₁H₃₈O₆=506.27)

The compound represented by the formula (4-2-X1-2): [M+Na]=735.3 (C₄₄H₅₆O₈=712.40)

The compound represented by the formula (4-2-X1-3): [M+Na]=941.6 (C₅₇H₇₄O₁₀=918.53)

(2) To a solution prepared by dissolving 5.0 g of the compound represented by the formula (4-2-X1-1) in 30 g of anhydrous N,N-dimethylformamide, 1.65 g of the compound represented by the formula (1A-1) was added. To the resultant mixture, 1.4 g of potassium carbonate and 0.03 g of tetrabutylammonium bromide were added. The obtained mixture was stirred at 80° C. for 6 hours. The reaction mixture obtained was cooled, and then, acidified (pH=3) with 100 g of 2% aqueous oxalic acid solution followed by conducting extraction with chloroform. The organic layer obtained was washed with pure water until becoming neutral. The organic layer was dried over anhydrous magnesium sulfate. The obtained mixture was filtrated and the filtrate was concentrated under reduced pressure to obtain 6.61 g of a resin, which is called as resin A17. Mw: 2.4×10³, Mw/Mn: 2.248, Yield: 83.8%.

### Example 18

To a solution prepared by dissolving 5.0 g of the compound represented by the formula (4-2-X1-2) in 20 g of anhydrous N,N-dimethylformamide, 1.17 g of the compound represented by the formula (1A-1) was added. To the resultant mixture, 1.0 g of potassium carbonate and 0.02 g of tetrabutylammonium bromide were added. The obtained mixture was stirred at 70 to 75° C. for 6 hours. The reaction mixture obtained was cooled, and then, acidified (pH=3) with 100 g of 2% aqueous oxalic acid solution followed by conducting extraction with ethyl acetate. The organic layer obtained was washed with pure water until becoming neutral. The organic layer was dried over anhydrous magnesium sulfate. The obtained mixture was filtrated and the filtrate was concentrated under reduced pressure to obtain 5.60 g of a resin, which is called as resin A18.

[M+Na]=1709.9 (C₁₀₃H₁₃₀O₂₀=1686.92), Yield: 94.6%.

### Example 19

(1) To a solution prepared by dissolving 6.2 g of the compound represented by the formula (7-1) in 60 g of anhydrous N,N-dimethylformamide, 10.0 g of the compound represented by the formula (X2) was added. To the resultant mixture, 8.1 g of potassium carbonate and 0.3 g of potassium iodide were added. The obtained mixture was stirred at 70° C. for 6 hours. The reaction mixture obtained was cooled, and then, acidified (pH=3) with 300 g of 3% aqueous oxalic acid solution followed by conducting extraction with ethyl acetate. The organic layer obtained was washed with pure water until becoming neutral. The organic layer was dried over anhydrous magnesium sulfate. The obtained mixture was filtrated and the filtrate was concentrated under reduced pressure to obtain 13.5 g of a mixture of the following compounds represented by the formulae (7-1-X2-1), (7-1-X2-2) and (7-1-X2-3):

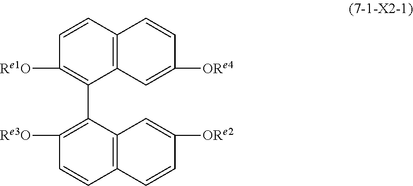

wherein any one of R^{e1}, R^{e2}, R^{e3} and R^{e4} is the group represented by the formula (X2-1) and the other three groups are hydrogen atoms,

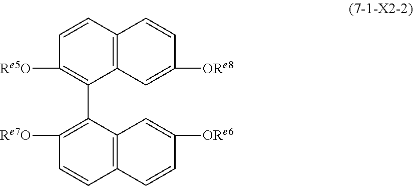

wherein any two of R^{e5}, R^{e6}, R^{e7} and R^{e8} are the groups represented by the formula (X2-1) and the other two groups are hydrogen atoms,

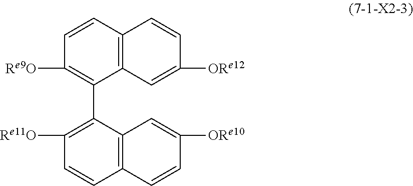

wherein any three of R^{e9}, R^{e10}, R^{e11} and R^{e12} are the groups represented by the formula (X2-1) and the other one group is a hydrogen atom, which is called as P6.

P6 was purified with silica gel column chromatography (hexane/ethyl acetate) to obtain 2.5 g of the compound represented by the formula (7-1-X2-1) (Yield: 23.8%), 3.0 g of the compound represented by the formula (7-1-X2-2) (Yield: 20.3%) and 1.2 g of the compound represented by the formula (7-1-X2-3) (Yield: 6.3%).

The compound represented by the formula (7-1-X2-1): [M+Na]=561.2 (C₃₄H₃₄O₆=538.24)

The compound represented by the formula (7-1-X2-2): [M+Na]=781.4 (C₄₈H₅₄O₈=758.38)

The compound represented by the formula (7-1-X2-3): [M+Na]=1001.5 (C₅₂H₇₄O₁₀=978.53)

(2) To a solution prepared by dissolving 2.0 g of the compound represented by the formula (7-1-X2-2) in 20 g of anhydrous N,N-dimethylformamide, 0.9 g of the compound represented by the formula (1A-1) was added. To the resultant mixture, 1.1 g of potassium carbonate was added. The obtained mixture was stirred at 80° C. for 8 hours. The reaction mixture obtained was cooled, and then, acidified (pH=3) with 100 g of 2% aqueous oxalic acid solution followed by conducting extraction with chloroform. The organic layer obtained was washed with pure water until becoming neutral. The organic layer was dried over anhydrous magnesium sulfate. The obtained mixture was filtrated and the filtrate was concentrated under reduced pressure to obtain 2.6 g of a resin, which is called as resin A19. Yield: 93.8%. Mw: 6.2×10³, Mw/Mn: 2.401.

### Reference Example 4

According to the method described in JP 2003-107708 A, a copolymer of p-hydroxystyrene and 2-ethyl-2-adamantyl methacrylate (molar ratio=20/80) and a copolymer of p-hydroxystyrene and 2-ethyl-2-adamantyl methacrylate (molar ratio=30/70) were prepared. The copolymers obtained were mixed in a weight ratio of 50/50 to prepare a resin, which is called as resin H1.

### Examples 20 to 25 and Comparative Example 1

### Resin

A1: resin A1
A2: resin A2
A19: resin A19
H1: resin H1

### <Acid Generator>

Acid generator B1: triphenylsulfonium 1-{3-(4-methylphenyl)adamantyl}methoxycarbonyldifluoromethane-sulfonate
Acid generator B2: triphenylsulfonium methoxycarbonyldifluoromethanesulfonate
Acid generator B3: triphenylsulfonium 2,4,6-triisopropylbenzenesulfonate
Acid generator B4: 4-methylphenyldiphenylsulfonium trifluoromethanesulfonate

### <Basic Compound (Quencher)>

Quencher C1: tris[2-(2-methoxyethoxy)ethyl]amine
Quencher C2: tris(2-hydroxy-3-propyl)amine
Quencher C3: tetrabutylammonium salicylate
Quencher C4: tetrabutylammonium hydroxide
Quencher C5: 2,6-diisopropylaniline

### <Solvent>

| | | |
|---|---|---|
| Solvent E1: | cyclohexanone | 700 parts |
| Solvent E2: | propylene glycol monomethyl ether acetate | 450 parts |
| | propylene glycol monomethyl ether | 100 parts |

In the following Examples, “part(s)” is on a weight basis.

The following components were mixed to give a solution, and the solution was further filtrated through a fluorine resin filter having a pore diameter of 0.2 μm, to prepare a photoresist composition.

Resin (kind and amount are described in Table 1)

Acid generator (kind and amount are described in Table 1)

Quencher (kind and amount are described in Table 1)

Solvent (kind are described in Table 1)

TABLE 1

| | Resin | Acid | | | |
|---|---|---|---|---|---|
| | (kind/ | generator | Quencher | | |
| | amount | (kind/amount | (kind/amount | | PB (° C.)/ |
| Ex. No. | (part)) | (part)) | (part)) | Solvent | PEB (° C.) |
| Ex. 20 | A2/10 | B2/2.5 | — | E1 | 110/110 |
| Ex. 21 | A2/10 | B1/4.0 | — | E1 | 110/110 |
| Ex. 22 | A2/10 | B2/2.5 | C1/0.25 | E1 | 110/110 |
| Ex. 23 | A1/10 | B1/1.5 | C1/0.25 | E1 | 110/130 |
| Ex. 24 | A19/10 | B3/3.0 | C2/0.1 | E1 | 110/110 |
| | | | C3/0.05 | | |
| Ex. 25 | A19/10 | B4/1.0 | C4/0.1 | E1 | 110/110 |
| | | B5/2.0 | C5/0.05 | | |
| Comp. | H1/10 | B1/1.5 | C1/0.1 | E2 | 110/100 |
| Ex. 1 | | | | | |

Silicon wafers were each contacted with hexamethyldisilazane at 90° C. for 60 seconds and each of the photoresist compositions prepared as above was spin-coated over the silicon wafer to give a film thickness after drying of 60 nm. After application of each of the resist compositions, the silicon wafers thus coated with the respective resist compositions were each prebaked on a direct hotplate at the temperature shown in column “PB” in Table 1 for 60 seconds. Using a writing electron beam lithography system (“HL-800D” manufactured by Hitachi, Ltd., 50 KeV), each wafer on which the respective photoresist film had been thus formed was exposed to a line and space pattern, while changing stepwise the exposure quantity.

After the exposure, each wafer was subjected to post-exposure baking on a hotplate at the temperature shown in column “PEB” in Table 1 for 60 seconds and then to paddle development with an aqueous solution of 2.38% by weight tetramethylammonium hydroxide for 60 seconds.

Each of a pattern developed on the silicon substrate after the development was observed with a scanning electron microscope, and the results of which are shown in Table 2.

Effective Sensitivity (ES): It is expressed as the amount of exposure that the line pattern and the space pattern become 1:1 after exposure through 200 nm line and space pattern mask and development.

Line Edge Roughness (LER): Each of a sidewall surface of 200 nm line and space pattern obtained at the exposure amount of the effective sensitivity was observed with a scanning electron microscope. When line edge roughness is very good and its evaluation is marked by “◯”, when line width roughness is good and its evaluation is marked by “Δ”, and when line width roughness is bad and its evaluation is marked by “X”.

TABLE 2

| Ex. No. | ES (μC) | LER |
|---|---|---|
| Ex. 20 | 50 | ◯ |
| Ex. 21 | 22 | ◯ |
| Ex. 22 | 50 | ◯ |
| Ex. 23 | 60 | ◯ |
| Ex. 24 | 16 | ◯ |
| Ex. 25 | 60 | ◯ |
| Comp. Ex. 1 | 15 | X |

Apparent from the results shown in Table 2, the photoresist compositions obtained by Examples corresponding to the present invention show good resolution and line edge roughness.

### Example 26

A photoresist composition can be prepared according to the same manner as described in Example 20, except that resin A3 is used in place of resin A2. A photoresist pattern can be obtained according to the same manner as described in Example 20, except that the photoresist composition containing resin A3 is used in place of the photoresist composition containing resin A2.

### Example 27

A photoresist composition can be prepared according to the same manner as described in Example 20, except that resin A4 is used in place of resin A2. A photoresist pattern can be obtained according to the same manner as described in Example 20, except that the photoresist composition containing resin A4 is used in place of the photoresist composition containing resin A2.

### Example 28

A photoresist composition can be prepared according to the same manner as described in Example 20, except that resin A5 is used in place of resin A2. A photoresist pattern can be obtained according to the same manner as described in Example 20, except that the photoresist composition containing resin A5 is used in place of the photoresist composition containing resin A2.

### Example 29

A photoresist composition can be prepared according to the same manner as described in Example 20, except that resin A6 is used in place of resin A2. A photoresist pattern can be obtained according to the same manner as described in Example 20, except that the photoresist composition containing resin A6 is used in place of the photoresist composition containing resin A2.

### Example 30

A photoresist composition can be prepared according to the same manner as described in Example 20, except that resin A7 is used in place of resin A2. A photoresist pattern can be obtained according to the same manner as described in Example 20, except that the photoresist composition containing resin A7 is used in place of the photoresist composition containing resin A2.

### Example 31

A photoresist composition can be prepared according to the same manner as described in Example 20, except that resin A8 is used in place of resin A2. A photoresist pattern can be obtained according to the same manner as described in Example 20, except that the photoresist composition containing resin A8 is used in place of the photoresist composition containing resin A2.

### Example 32

A photoresist composition can be prepared according to the same manner as described in Example 20, except that resin A9 is used in place of resin A2. A photoresist pattern can be obtained according to the same manner as described in Example 20, except that the photoresist composition containing resin A9 is used in place of the photoresist composition containing resin A2.

### Example 33

A photoresist composition can be prepared according to the same manner as described in Example 20, except that resin A10 is used in place of resin A2. A photoresist pattern can be obtained according to the same manner as described in Example 20, except that the photoresist composition containing resin A10 is used in place of the photoresist composition containing resin A2.

### Example 34

A photoresist composition can be prepared according to the same manner as described in Example 20, except that resin All is used in place of resin A2. A photoresist pattern can be obtained according to the same manner as described in Example 20, except that the photoresist composition containing resin All is used in place of the photoresist composition containing resin A2.

### Example 35

A photoresist composition can be prepared according to the same manner as described in Example 20, except that resin A12 is used in place of resin A2. A photoresist pattern can be obtained according to the same manner as described in Example 20, except that the photoresist composition containing resin A12 is used in place of the photoresist composition containing resin A2.

### Example 36

A photoresist composition can be prepared according to the same manner as described in Example 20, except that resin A13 is used in place of resin A2. A photoresist pattern can be obtained according to the same manner as described in Example 20, except that the photoresist composition containing resin A13 is used in place of the photoresist composition containing resin A2.

### Example 37

A photoresist composition can be prepared according to the same manner as described in Example 20, except that resin A14 is used in place of resin A2. A photoresist pattern can be obtained according to the same manner as described in Example 20, except that the photoresist composition containing resin A14 is used in place of the photoresist composition containing resin A2.

### Example 38

A photoresist composition can be prepared according to the same manner as described in Example 20, except that resin A15 is used in place of resin A2. A photoresist pattern can be obtained according to the same manner as described in Example 20, except that the photoresist composition containing resin A15 is used in place of the photoresist composition containing resin A2.

### Example 39

A photoresist composition can be prepared according to the same manner as described in Example 20, except that resin A16 is used in place of resin A2. A photoresist pattern can be obtained according to the same manner as described in Example 20, except that the photoresist composition containing resin A16 is used in place of the photoresist composition containing resin A2.

### Example 40

A photoresist composition can be prepared according to the same manner as described in Example 20, except that resin A17 is used in place of resin A2. A photoresist pattern can be obtained according to the same manner as described in Example 20, except that the photoresist composition containing resin A17 is used in place of the photoresist composition containing resin A2.

### Example 41

A photoresist composition can be prepared according to the same manner as described in Example 20, except that resin A18 is used in place of resin A2. A photoresist pattern can be obtained according to the same manner as described in Example 20, except that the photoresist composition containing resin A18 is used in place of the photoresist composition containing resin A2.

The present resist composition provides excellent photoresist pattern in resolution and line edge roughness, and is suitable for EUV lithography and EB lithography.

1. A resin obtained by reacting a compound represented by the formula (I): 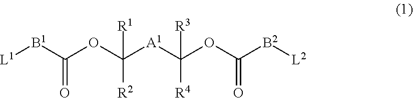
wherein R¹ represents a hydrogen atom or a C1-C6 alkyl group, R², R³ and R⁴ each independently represents a C1-C6 alkyl group and R¹, R², R³, R⁴ and A¹ are bonded to form a ring, A¹ represents a C1-C20 saturated hydrocarbon group in which one or more —CH₂— can be replaced by —O—, B¹ and B² each independently represent a C1-C6 alkylene group, L¹ and L² each independently represent a halogen atom, —O—CH═CH₂, —O—CH═CH(CH₃) or —O—SO₂—R′ in which R′ represents a C1-C6 alkyl group or a C6-C20 aryl group, with a polyhydric phenol compound.

2. The resin according to claim 1, wherein R′ is a methyl group or a tolyl group.

3. The resin according to claim 1 or 2, wherein the polyhydric phenol compound is at least one selected from the group consisting of compounds represented by the formulae (2) and (4) to (7): 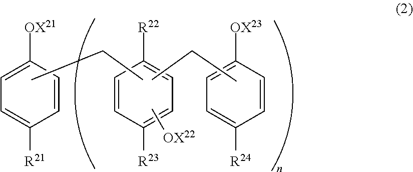
wherein R²¹, R²², R²³ and R²⁴ each independently represent a hydrogen atom, —OX²⁴ or a C1-C6 alkyl group, n represents an integer of 0 to 3, X²¹, X²², X²³ and X²⁴ each independently represent a hydrogen atom or a group represented by the formula (3): 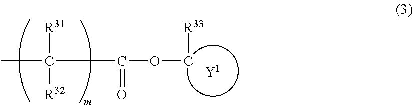
wherein R³¹ and R³² each independently represent a hydrogen atom or a C1-C6 alkyl group, m represents an integer of 1 to 4, R³³ represents a C1-C6 alkyl group or a C3-C12 saturated cyclic hydrocarbon group, and ring Y¹ represents a C3-C20 saturated hydrocarbon ring, and at least two selected from the group consisting of X²¹, X²², X²³ and X²⁴ are hydrogen atoms, 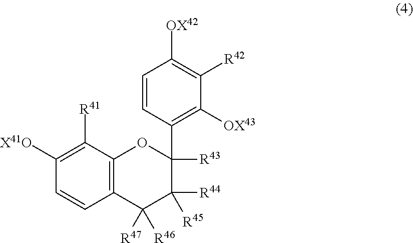
wherein R⁴¹, R⁴², R⁴³, R⁴⁴, R⁴⁵, R⁴⁶ and R⁴⁷ each independently represents a hydrogen atom, —OX⁴⁴ or a C1-C6 alkyl group, and R⁴³ and R⁴⁴ can be bonded each other to form a C3-C20 ring together with the carbon atoms to which they are bonded, and R⁴⁶ and R⁴⁷ can be bonded each other to form a C3-C20 ring together with the carbon atom to which they are bonded, X⁴¹, X⁴², X⁴³ and X⁴⁴ each independently represent a hydrogen atom or the group represented by the formula (3), and at least two selected from the group consisting of X⁴¹, X⁴², X⁴³ and X⁴⁴ are hydrogen atoms, 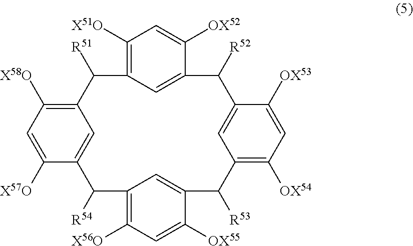
wherein R⁵¹, R⁵², R⁵³ and R⁵⁴ each independently represents a hydrogen atom, —OX⁵⁹, a C1-C6 alkyl group, a C3-C10 cycloalkyl group, a C4-C20 cycloalkylalkyl group, a C6-C20 aryl group or a C7-C20 aralkyl group, and the alkyl group, the aryl group and the aralkyl group may be substituted with —OX⁶⁰, and X⁵¹, X⁵², X⁵³, X⁵⁴, X⁵⁵, X⁵⁶, X⁵⁷, X⁵⁸, X⁵⁹ and X⁶⁰ each independently represent a hydrogen atom or the group represented by the formula (3), and at least two selected from the group consisting of X⁵¹, X⁵², X⁵³, X⁵⁴, X⁵⁵, X⁵⁶, X⁵⁷, X⁵⁸, X⁵⁹ and X⁶⁰ are hydrogen atoms, 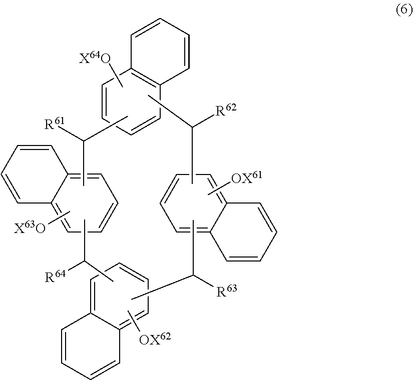
wherein R⁶¹, R⁶², R⁶³ and R⁶⁴ each independently represents a hydrogen atom, —OX⁶⁵, a C1-C6 alkyl group, a C3-C10 cycloalkyl group, a C4-C20 cycloalkylalkyl group, a C6-C20 aryl group or a C7-C20 aralkyl group, and the alkyl group, the aryl group and the aralkyl group may be substituted with —OX⁶⁶, and X⁶¹, X⁶², X⁶³, X⁶⁴, X⁶⁵ and X⁶⁶ each independently represent a hydrogen atom or the group represented by the formula (3), and at least two selected from the group consisting of X⁶¹, X⁶², X⁶³, X⁶⁴, X⁶⁵ and X⁶⁶ are hydrogen atoms, 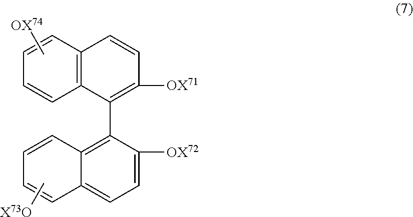
wherein X⁷¹, X⁷², X⁷³ and X⁷⁴ each independently represent a hydrogen atom or the group represented by the formula (3), and at least two selected from the group consisting of X⁷¹, X⁷², X⁷³ and X⁷⁴ are hydrogen atoms.

4. The resin according to claim 1, wherein the compound represented by the formula (1) is a compound represented by the formula (10): 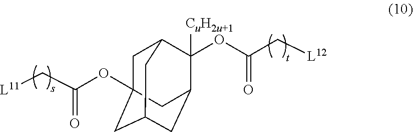
wherein s, t and u each independently represent an integer of 1 to 6, L¹¹ and L¹² each independently represent a halogen atom, —O—CH═CH₂, —O—CH═CH(CH₃) or —O—SO₂—R″ in which R″ represents a C1-C6 alkyl group or a C6-C20 aryl group.

5. The resin according to claim 1, wherein the resin is obtained by reacting a compound represented by the formula (10) with a polyhydric phenol compound.

6. A compound represented by the formula (10): 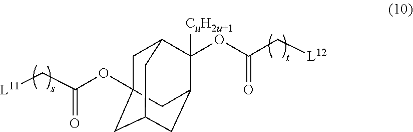
wherein s, t and u each independently represent an integer of 1 to 6, L¹¹ and L¹² each independently represent a halogen atom, —O—CH═CH₂, —O—CH═CH(CH₃) or —O—SO₂—R″ in which R″ represents a C1-C6 alkyl group or a C6-C20 aryl group.

7. A process for producing a resin comprising reacting a compound represented by the formula (I): 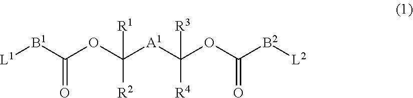
wherein R¹ represents a hydrogen atom or a C1-C6 alkyl group, R², R³ and R⁴ each independently represents a C1-C6 alkyl group and R¹, R², R³, R⁴ and A¹ are bonded to form a ring, A¹ represents a C1-C20 saturated hydrocarbon group in which one or more —CH₂— can be replaced by —O—, B¹ and B² each independently represent a C1-C6 alkylene group, L¹ and L² each independently represent a halogen atom, —O—CH═CH₂, —O—CH═CH(CH₃) or —O—SO₂—R′ in which R′ represents a C1-C6 alkyl group or a C6-C20 aryl group, with a polyhydric phenol compound.

8. A photoresist composition comprising the resin according to claim 1 and an acid generator.

9. The photoresist composition according to claim 8, wherein the acid generator is an acid generator represented by the formula (B1): 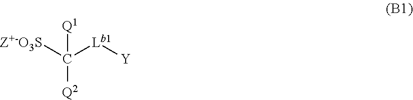
wherein Q¹ and Q² each independently represent a fluorine atom or a C1-C6 perfluoroalkyl group, L^{b1} represents a single bond or a C1-C17 saturated divalent hydrocarbon group in which one or more —CH₂— can be replaced by —O— or —CO—, Y represents a C1-C18 aliphatic hydrocarbon group or a C3-C18 saturated cyclic hydrocarbon group, and the aliphatic hydrocarbon group and the saturated cyclic hydrocarbon group can have one or more substituents, and one or more —CH₂— in the aliphatic hydrocarbon group and the saturated cyclic hydrocarbon group can be replaced by —O—, —CO— or —SO₂—, and Z⁺ represents an organic cation.
